(19) United States
(12) Patent Application Publication
(10) Pub. No.: US 2024/0099836 A1
(43) Pub. Date: Mar. 28, 2024

(54) PROSTHETIC VALVE ASSEMBLIES

(71) Applicant: EDWARDS LIFESCIENCES CORPORATION, Irvine, CA (US)

(72) Inventors: David Maimon, Atlit (IL); Tamir S. Levi, Zikhron Yaakov (IL); Ziv Yohanan, Kfar Hahoresh (IL); Anatoly Dvorsky, Haifa (IL)

(21) Appl. No.: 18/531,385

(22) Filed: Dec. 6, 2023

Related U.S. Application Data
(63) Continuation of application No. PCT/US2022/033384, filed on Jun. 14, 2022.
(60) Provisional application No. 63/348,085, filed on Jun. 2, 2022.
(60) Provisional application No. 63/210,646, filed on Jun. 15, 2021.

(51) Int. Cl. A61F 2/24

(52) U.S. Cl. CPC A61F 2/2418; A61F 2002/8483

(57) ABSTRACT

Devices, assemblies, and methods for displacement of leaflets surrounding a prosthetic valve, such as native leaflets or host leaflets of a previously-implanted prosthetic valve, during valve implantation procedures are disclosed herein. As one example, a valve assembly can include a prosthetic valve and a leaflet engagement frame coupled to the frame of the prosthetic valve. The leaflet engagement frame can include a plurality of engagement frame struts defining one or more rungs, and a plurality of spikes that extend in a distal direction from a first rung of struts of the leaflet engagement frame. The distally extending spikes are configured to engage with one or more native or host leaflets positioned outside the valve assembly when the valve assembly is in a partially expanded state, and to distally fold the engaged native or host leaflets when the valve assembly is further expanded.

### CROSS REFERENCE TO RELATED APPLICATIONS

This application is a continuation of International Application No. PCT/US2022/033384, filed Jun. 14, 2022, which claims benefit of U.S. Provisional Application No. 63/210,646, filed on Jun. 15, 2021, and U.S. Provisional Application No. 63/348,085, filed on Jun. 2, 2022, the contents of each of which are herein incorporated by reference in their entirety.

### FIELD

The present disclosure relates to devices, assemblies and methods for displacement of leaflets surrounding a prosthetic valve, such as native leaflets or host leaflets of a previously-implanted prosthetic valve, during valve implantation procedures.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Since surgeries are prone to an abundance of clinical complications, alternative less invasive techniques of delivering a prosthetic heart valve over a catheter and implanting it over the native malfunctioning valve have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

During implantation procedures of such prosthetic valves within native heart valves, for example, the native leaflets may be pushed aside by the expansion of the prosthetic valve, with the prosthetic leaflets performing the function previously provided by the native leaflets. This in turn may pose a risk of coronary artery obstruction. In particular, the native leaflets may block the coronary artery ostia or otherwise inhibit blood flow through the frame of the prosthetic vale to the coronary artery ostia. A similar problem may occur in Valve-in-Valve (ViV) procedures, during which a new prosthetic valve is mounted within a previously-implanted prosthetic valve, for example, when the leaflets of the previously-implanted prosthetic valve are similarly pushed sideways, posing a risk of coronary artery obstruction.

Existing methods for prevention of coronary access obstruction rely on lacerating existing leaflets, and require high spatial precision and surgical skill. Moreover, portions of the lacerated leaflet may still act to partially or completely obstruct the coronary ostia. Improvements accordingly may be desired in addressing positions of existing leaflets within a patient's body during prosthetic valve implantation, to avoid coronary artery obstruction.

### SUMMARY

According to an aspect of the disclosure, there is provided a valve assembly comprising a prosthetic valve and a leaflet engagement frame which is disposed radially outwardly from, and is coupled to, a valve frame of the prosthetic valve. The valve frame comprises a plurality of struts, and is movable between a radially compressed state and a radially expanded state. The prosthetic valve can further include a leaflet assembly mounted within the valve frame and comprising a plurality of leaflets configured to regulate flow through the prosthetic valve.

The leaflet engagement frame comprises a plurality of engagement frame struts defining one or more rungs of struts. The one or more rungs of struts comprises an engagement frame first rung. The leaflet engagement frame further comprises a plurality of distally extending spikes, extending in the distal direction solely from the struts of the engagement frame first rung.

The plurality of distally extending angled spikes is configured to engage with one or more native or host leaflets positioned outside the valve assembly when the valve assembly is in a partially expanded state, and to distally fold the engaged native or host leaflets when the valve assembly is further expanded.

In some examples, the plurality of distally extending spikes extend at an angle α radially outward and in the distal direction from the respective struts of the engagement frame first rung.

In some examples, the valve assembly further comprises an outer skirt comprising an outer skirt outflow end and an outer skirt inflow end, wherein the outer skirt is mounted over an outer surface of the valve assembly. In some examples, the outer skirt is mounted over a portion of an outer surface of the valve frame, and/or a portion of an outer surface of the leaflet engagement frame.

In some examples, the leaflet engagement frame is devoid of any other rungs of struts other than the engagement frame first rung.

In some examples, the outer skirt outflow end is distal to the plurality of distally extending spikes.

In some examples, the one or more rungs of struts of the leaflet engagement frame comprises at least one additional rung of struts distal to the engagement frame first rung, wherein the leaflet engagement frame further comprises a plurality of proximally extending spikes, extending in the proximal direction from the engagement frame struts of the additional rung of struts.

In some examples, the plurality of proximally extending spikes extend at an angle β radially outward and in the proximal direction from the additional rung of struts.

In some examples, the at least one additional rung of struts comprising the proximally extending spikes is an engagement frame second rung, and the leaflet engagement frame is devoid of any other rungs of struts other than the engagement frame first rung and the engagement frame second rung.

In some examples, the outer skirt outflow end is distal to the distally extending spikes and the proximally extending spikes.

In some examples, the one or more rungs of struts of the leaflet engagement frame further comprises one or more intermediate rungs of struts disposed between the rungs of struts that include the distally extending spikes and the proximally extending spikes, wherein any of the one or more intermediate rungs of struts is devoid of spikes.

In some examples, the outer skirt outflow end is distal to the distally extending spikes, and the outer skirt inflow end is proximal to the proximally extending spikes.

In some examples, each proximally extending spike terminates with a sharp distal tip.

In some examples, the angle β is in the range of 10-80 degrees.

In some examples, the angle β is in the range of 20-70 degrees.

In some examples, the angle β is in the range of 30-60 degrees.

In some examples, each distally extending spike terminates with a sharp distal tip.

In some examples, the angle α is in the range of 10-80 degrees.

In some examples, the angle α is in the range of 20-70 degrees.

In some examples, the angle α is in the range of 30-60 degrees.

In some examples, the plurality of struts of the valve frame define a plurality of rungs of struts that include: a valve frame outflow rung, a valve frame inflow rung, and at least one valve frame intermediate rung disposed between the valve frame outflow rung and the valve frame inflow rung. The struts of the engagement frame first rung are aligned with struts of a valve frame intermediate rung which is distal to the valve frame outflow rung.

In some examples, the at least one valve frame intermediate rung comprises: a valve frame first intermediate rung, a valve frame second intermediate rungs, and a valve frame third intermediate rungs, wherein the struts of the valve frame further comprise valve frame distal axial struts interconnecting struts of the valve frame inflow rung with struts of the valve frame third intermediate rung, and wherein the struts of the engagement frame first rung are aligned with the struts of the valve frame first intermediate rung.

In some examples, the at least one valve frame intermediate rung comprises a valve frame first intermediate rung and a valve frame second intermediate rung, wherein the struts of the engagement frame first rung are aligned with the struts of the valve frame first intermediate rung.

In some examples, the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.

According to another aspect of the disclosure, there is provided a leaflet engagement frame for a valve assembly, the leaflet engagement frame comprising a plurality of engagement frame struts defining one or more rungs of struts. The one or more rungs of struts comprises an engagement frame first rung. The leaflet engagement frame further comprises a plurality of distally extending spikes, extending in the distal direction solely from the struts of the engagement frame first rung.

The plurality of distally extending angled spikes are configured to engage with one or more native or host leaflets positioned outside the leaflet engagement frame when the leaflet engagement frame is in a partially expanded state, and to distally fold the engaged native or host leaflets when the leaflet engagement frame is further expanded.

In some examples, the distally extending spikes are extending at a sharp angle α radially outward and in the distal direction from the respective struts of the engagement frame first rung.

In some examples, the leaflet engagement frame is devoid of any other rungs of struts other than the engagement frame first rung.

In some examples, the one or more rungs of struts comprises at least one additional rung of struts distal to the engagement frame first rung, wherein the leaflet engagement frame further comprises a plurality of proximally extending spikes, extending in the proximal direction from the engagement frame struts of the additional rung of struts.

In some examples, the proximally extending spikes are extending at a sharp angle β radially outward and in the proximal direction from the additional rung of struts.

In some examples, the at least one additional rung of struts comprising the proximally extending spikes is an engagement frame second rung, wherein the leaflet engagement frame is devoid of any other rungs of struts other than the engagement frame first rung and the engagement frame second rung.

In some examples, the leaflet engagement frame further comprises one or more intermediate rungs of struts disposed between the rungs of struts that include the distally extending spikes and the proximally extending spikes, wherein any of the one or more intermediate rungs of struts is devoid of spikes.

In some examples, each proximally extending spike terminates with a sharp distal tip.

In some examples, the angle β is in the range of 10-80 degrees.

In some examples, the angle β is in the range of 20-70 degrees.

In some examples, the angle β is in the range of 30-60 degrees.

In some examples, each distally extending spike terminates with a sharp distal tip.

In some examples, the angle α is in the range of 10-80 degrees.

In some examples, the angle α is in the range of 20-70 degrees.

In some examples, the angle α is in the range of 30-60 degrees.

According to another aspect of the disclosure, there is provided a valve assembly comprising a prosthetic valve and at least one proximal leaflet engagement wire. The prosthetic valve comprises a valve frame movable between a radially compressed state and a radially expanded state, and a leaflet assembly mounted within the valve frame. The valve frame comprises a plurality of frame rungs, each frame rung including interconnected struts. The plurality of frame rungs includes: a valve frame outflow rung, a valve frame inflow rung, and one or more valve frame intermediate rungs disposed between the valve frame outflow rung and the valve frame inflow rung. The valve frame outflow rung is proximal to any other rung of the plurality of frame rungs. The valve frame inflow rung is distal to any other rung of the plurality of frame rungs. The at least one valve frame intermediate rung comprising a valve frame first intermediate rung.

The leaflet assembly comprises a plurality of leaflets configured to regulate flow through the prosthetic valve. The at least one proximal leaflet engagement wire is wrapped around struts of a at least one of the one or more valve frame intermediate rungs. The at least one proximal leaflet engagement wire is configured to engage with one or more native or host leaflets positioned outside the valve assembly when the valve assembly is in a partially expanded state, and to distally fold the engaged native or host leaflets when the valve assembly is further expanded.

In some examples, the valve assembly further comprises an outer skirt comprising an outer skirt outflow end and an outer skirt inflow end, wherein the outer skirt is mounted over an outer surface of the valve assembly.

In some examples, the at least one proximal leaflet engagement wire comprises a single continuous proximal leaflet engagement wire, wrapped around all struts of the valve frame intermediate rung.

In some examples, valve frame intermediate rung around which the at least one proximal leaflet engagement wire is wrapped, comprises a single rung of the one or more valve frame intermediate rungs.

In some examples, all other frame rungs are devoid of engagement wires wrapped there-over.

In some examples, the outer skirt outflow end is distal to the proximal leaflet engagement wire.

In some examples, the valve assembly further comprises at least one distal leaflet engagement wire wrapped around struts of at least one other rung of the plurality of frame rungs, wherein the at least one other rung is distal to the proximal leaflet engagement wire.

In some examples, the at least one other rung comprises a single rung of the plurality of frame rungs.

In some examples, all other valve frame rungs, except the single rung around which the proximal leaflet engagement wire is wrapped and the single rung around which the distal leaflet engagement wire is wrapped, are devoid of wires wrapped there-over.

In some examples, the at least one other rung around which the distal leaflet engagement wire is wrapped comprises the valve frame inflow rung.

In some examples, the outer skirt outflow end is distal to the proximal leaflet engagement wire, and the outer skirt inflow end is proximal to the distal leaflet engagement wire.

In some examples, the at least one valve frame intermediate rung further comprises a valve frame second intermediate rung, wherein the at least one other rung around which the distal leaflet engagement wire is wrapped comprises the valve frame second intermediate rung.

In some examples, the outer skirt outflow end is distal to the proximal leaflet engagement wire and the distal leaflet engagement wire.

In some examples, the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.

According to another aspect of the disclosure, there is provided a prosthetic valve comprising a valve frame movable between a radially compressed state and a radially expanded state, and a leaflet assembly mounted within the valve frame. The valve frame comprises a plurality of rungs of interconnected struts, that include: a valve frame outflow rung, a valve frame inflow rung, and at least one valve frame intermediate rung disposed between the valve frame outflow rung and the valve frame inflow rung. The at least one valve frame intermediate rung comprising a valve frame first intermediate rung. The leaflet assembly comprises a plurality of leaflets configured to regulate flow through the prosthetic valve.

The prosthetic valve further comprises a plurality of distally extending spikes, integrally formed with the struts of a single valve frame intermediate rung, and extending in the distal direction therefrom. The plurality of distally extending spikes are configured to engage with one or more native or host leaflets positioned outside the prosthetic valve when the prosthetic valve is in a partially expanded state, and to distally fold the engaged native or host leaflets when the prosthetic valve is further expanded.

In some examples, the distally extending spikes are extending at a sharp angle α radially outward and in the distal direction from the respective struts of the engagement frame first rung.

In some examples, the prosthetic valve further comprises an outer skirt comprising an outer skirt outflow end and an outer skirt inflow end, wherein the outer skirt is mounted over an outer surface of the prosthetic valve.

In some examples, the outer skirt outflow end is distal to the distally extending spikes.

In some examples, the prosthetic valve further comprises a plurality of proximally extending spikes integrally formed with another single valve frame rung which is distal to the distally extending spike, and extending in the proximal direction therefrom.

In some examples, the proximally extending spikes are extending at a sharp angle β radially outward and in the proximal direction from the additional rung of struts.

In some examples, all other valve frame rungs, except the single rung comprising the distally extending spikes and the single rung comprising the proximally extending spikes, are devoid of integrally formed spikes

In some examples, the proximally extending spikes are integrally formed with struts of the valve frame inflow rung.

In some examples, the outer skirt outflow end is distal to the distally extending spikes, and the outer skirt inflow end is proximal to the proximally extending spikes.

In some examples, the at least one valve frame intermediate rung further comprises a valve frame second intermediate rung, and wherein the proximally extending spikes are integrally formed with struts of the valve frame second intermediate rung.

In some examples, the outer skirt outflow end is distal to the distally extending spikes and the proximally extending spikes.

In some examples, the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.

In some examples, each proximally extending spike terminates with a sharp distal tip.

In some examples, the angle β is in the range of 10-80 degrees.

In some examples, the angle β is in the range of 20-70 degrees.

In some examples, the angle β is in the range of 30-60 degrees.

In some examples, each distally extending spikes terminates with a sharp distal tip.

In some examples, the angle α is in the range of 10-80 degrees.

In some examples, the angle α is in the range of 20-70 degrees.

In some examples, the angle α is in the range of 30-60 degrees.

According to another aspect of the disclosure, there is provided a method that comprises positioning a valve assembly between native or host leaflets within a patient's body, wherein the valve assembly comprises a prosthetic valve and a leaflet engagement frame. The prosthetic valve comprises a valve frame movable between a radially compressed state and a radially expanded state, and a leaflet assembly mounted within the valve frame. The leaflet assembly comprises a plurality of leaflets configured to regulate flow through the prosthetic valve.

The leaflet engagement frame is disposed radially outwardly from, and is coupled to, the valve frame. The leaflet engagement frame comprises a plurality of distally extending angled spikes, extending at a sharp angle α radially outward and in the distal direction solely from struts of an engagement frame first rung of the leaflet engagement frame.

The method further comprises partially expanding the valve assembly at least until the distally extending angled spikes engage with the native or host leaflets.

The method further comprises further expanding the valve assembly such that the distally extending angled spikes remain engaged with the native or host leaflets, and drag the native or host leaflets distally to a folded configuration of the native or host leaflets.

In some examples, the leaflet engagement frame is devoid of any other rungs of struts other than the engagement frame first rung.

In some examples, the method further comprises a step of advancing the valve assembly in a distal direction after the step of partially expanding the valve assembly, such that the distally extending angled spikes remain engaged with the native or host leaflets and drag the native or host leaflets along with the valve assembly during its advancement in the partially expanded state.

In some examples, the leaflet engagement frame further comprises a plurality of proximally extending angled spikes, extending at a sharp angle β radially outward and in the proximal direction solely from struts of an additional rung of the leaflet engagement frame, distal to the distally extending angled spikes.

In some examples, the step of partially expanding the valve assembly comprises partially expanding the valve assembly at least until the proximally extending angled spikes engage with a native annulus or with the native or host leaflets.

In some examples, the leaflet engagement frame comprises additional rungs of struts between the distally extending angled spikes and the proximally extending angled spikes, and wherein the additional rungs of struts are devoid of angled spikes.

In some examples, each proximally extending angled spike terminates with a sharp distal tip.

In some examples, the angle β is in the range of 10-80 degrees.

In some examples, the angle β is in the range of 20-70 degrees.

In some examples, the angle β is in the range of 30-60 degrees.

In some examples, each distally extending angled spike terminates with a sharp distal tip.

In some examples, the angle α is in the range of 10-80 degrees.

In some examples, the angle α is in the range of 20-70 degrees.

In some examples, the angle α is in the range of 30-60 degrees.

According to another aspect of the disclosure, there is provided a method that comprises positioning a valve assembly between native or host leaflets within a patient's body, wherein the valve assembly comprises a prosthetic valve and at least one proximal leaflet engagement wire. The prosthetic valve comprises a valve frame movable between a radially compressed state and a radially expanded state, and a leaflet assembly mounted within the valve frame.

The valve frame comprises a valve frame outflow rung, a valve frame inflow rung, and at least one valve frame intermediate rung disposed between the valve frame outflow rung and the valve frame inflow rung. The at least one proximal leaflet engagement wire is wrapped around struts of a single valve frame intermediate rung. The leaflet assembly comprises a plurality of leaflets configured to regulate flow through the prosthetic valve.

The method further comprises partially expanding the valve assembly at least until the proximal leaflet engagement wire engages with the native or host leaflets.

The method further comprises further expanding the valve assembly such that the proximal leaflet engagement wire remains engaged with the native or host leaflets, and drags the native or host leaflets distally to a folded configuration of the native or host leaflets.

In some examples, the at least one proximal leaflet engagement wire comprises a single continuous proximal leaflet engagement wire, wrapped around all struts of the valve frame intermediate rung.

In some examples, all other rungs of struts of the valve frame are devoid of wires wrapped there-over.

In some examples, the method further comprises a step of advancing the valve assembly in a distal direction after the step of partially expanding the valve assembly, such that the proximal leaflet engagement wire remains engaged with the native or host leaflets and drags the leaflets along with the valve assembly during its advancement in the partially expanded state.

In some examples, the leaflet engagement frame further comprises at least one distal leaflet engagement wire wrapped around struts of another single valve frame rung which is distal to the proximal leaflet engagement wire.

In some examples, the at least one distal leaflet engagement wire comprises a single distal leaflet engagement wire, wrapped around all struts of the valve frame rung.

In some examples, the step of partially expanding the valve assembly comprises partially expanding the valve assembly at least until the distal leaflet engagement wire engages with a native annulus or with the native or host leaflets.

In some examples, all other valve frame rungs, except the single rung around which the proximal leaflet engagement wire is wrapped and the single rung around which the distal leaflet engagement wire is wrapped, are devoid of wires wrapped there-over.

In some examples, the distal leaflet engagement wire is wrapped around struts of the valve frame inflow rung.

In some examples, the at least one valve frame intermediate rung further comprises a valve frame second intermediate rung, and wherein the distal leaflet engagement wire is wrapped around struts of the valve frame second intermediate rung.

According to another aspect of the disclosure, there is provided a method that comprises positioning a prosthetic valve between native or host leaflets within a patient's body, wherein the prosthetic valve comprises a valve frame movable between a radially compressed state and a radially expanded state, and a leaflet assembly mounted within the valve frame. The valve frame comprises a valve frame outflow rung, a valve frame inflow rung, and at least one valve frame intermediate rung disposed between the valve frame outflow rung and the valve frame inflow rung.

The prosthetic valve further comprises a plurality of distally extending angled spikes, integrally formed with struts of a single valve frame intermediate rung, and extending at a sharp angle α radially outward and in the distal direction therefrom. The leaflet assembly comprises a plurality of leaflets configured to regulate flow through the prosthetic valve.

The method further comprises partially expanding the prosthetic valve at least until the distally extending angled spikes engage with the native or host leaflets.

The method further comprises further expanding the prosthetic valve such that the distally extending angled spikes remain engaged with the native or host leaflets, and drag the native or host leaflets distally to a folded configuration of the native or host leaflets.

In some examples, all other rungs of the valve frame are devoid of angled spikes.

In some examples, the method further comprises a step of advancing the prosthetic valve in a distal direction after the step of partially expanding the prosthetic valve, such that the distally extending angled spikes remain engaged with the native or host leaflets and drag the native or host leaflets along with the prosthetic valve during its advancement in the partially expanded state.

In some examples, the prosthetic valve further comprises a plurality of proximally extending angled spikes integrally formed with another single valve frame rung which is distal to the distally extending angled spike, and extending at a sharp angle β radially outward and in the proximal direction therefrom.

In some examples, the step of partially expanding the prosthetic valve comprises partially expanding the valve assembly at least until the proximally extending angled spikes engage with a native annulus or with the native or host leaflets.

In some examples, all other valve frame rungs, except the single rung comprising the distally extending angled spikes and the single rung comprising the proximally extending angled spikes, are devoid of integrally formed spikes.

In some examples, the proximally extending angled spikes are integrally formed with struts of the valve frame inflow rung.

In some examples, the at least one valve frame intermediate rung further comprises a valve frame second intermediate rung, wherein the proximally extending angled spikes are integrally formed with struts of the valve frame second intermediate rung.

In some examples, each proximally extending angled spike terminates with a sharp distal tip.

In some examples, the angle β is in the range of 10-80 degrees.

In some examples, the angle β is in the range of 20-70 degrees.

In some examples, the angle β is in the range of 30-60 degrees.

In some examples, each distally extending angled spike terminates with a sharp distal tip.

In some examples, the angle α is in the range of 10-80 degrees.

In some examples, the angle α is in the range of 20-70 degrees.

In some examples, the angle α is in the range of 30-60 degrees.

According to another aspect of the disclosure, there is provided a method that comprises positioning a valve assembly between native or host leaflets within a patient's body, wherein the valve assembly comprises a prosthetic valve and a leaflet engagement frame. The prosthetic valve comprises a valve frame movable between a radially compressed state and a radially expanded state, and a leaflet assembly mounted within the valve frame. The leaflet assembly comprises a plurality of leaflets configured to regulate flow through the prosthetic valve.

The leaflet engagement frame is disposed radially outwardly from, and is coupled to, the valve frame. The leaflet engagement frame comprises a plurality of distally extending angled spikes, extending at a sharp angle α radially outward and in the distal direction solely from struts of an engagement frame first rung of the leaflet engagement frame.

The method further comprises partially expanding the valve assembly.

The method further comprises confirming that the valve assembly is in proper position in the patient's body using tactile feedback from the valve assembly created by contact of the distally extending angled spikes with the native or host leaflets.

The method further comprises further expanding the valve assembly in a desired position within the patient's body.

In some examples, the leaflet engagement frame is devoid of any other rungs of struts other than the engagement frame first rung.

In some examples, each distally extending angled spike terminates with a sharp distal tip.

In some examples, the angle α is in the range of 10-80 degrees.

In some examples, the angle α is in the range of 20-70 degrees.

In some examples, the angle α is in the range of 30-60 degrees.

According to another aspect of the disclosure, there is provided a valve assembly comprising a prosthetic valve and a leaflet engagement frame. The prosthetic valve comprises a valve frame movable between a radially compressed state and a radially expanded state, and a leaflet assembly mounted within the valve frame. The valve frame is defined between a valve outflow end and a valve inflow end, and comprises intersecting struts that define at least one row of circumferentially extending frame cells. The leaflet assembly comprises a plurality of leaflets configured to regulate flow through the prosthetic valve.

The leaflet engagement frame which is disposed radially outwardly from, and is coupled to, the valve frame. The leaflet engagement frame comprises first struts defined along an engagement frame first rung, and second struts defined along an engagement frame second rung, distal to the engagement frame first rung. The leaflet engagement frame further comprises three engagement cells, each engagement cell defined by two first struts extending from an engagement frame outflow apex to engagement frame intermediate junctions, and two second struts extending from the two engagement frame intermediate junctions to an engagement frame inflow apex.

The leaflet engagement frame further comprises three distally extending hooks, wherein each distally extending hook comprises a curved portion connected on one end to a corresponding engagement frame outflow apex, and an opposite free end positioned radially away from the corresponding outflow apex and oriented in a distal direction. The distally extending hooks are configured to capture, in the curved portions, proximal ends of one or more native or host leaflets, positioned outside the valve assembly, when the valve assembly is in a partially expanded state, and to distally fold the captured native or host leaflets when the valve assembly is further expanded.

In some examples, the valve frame comprises a row with more than three cells. In some examples, the valve frame comprises a row with six cells. In some examples, the valve frame comprises a row with nine cells. In some examples, the valve frame comprises a row with twelve cells. Any example of a row of the valve frame comprising more than three cells, refers to a row underlying at least a portion of the leaflet engagement frame.

In some examples, the first struts and the second struts of the leaflet engagement frame are not aligned with any of the struts of the valve frame.

In some examples, the second struts are longer than the first struts.

In some examples, the valve assembly further comprises an outer skirt comprising an outer skirt outflow end and an outer skirt inflow end, wherein the outer skirt is mounted over an outer surface of the valve assembly such that the outer skirt outflow end is distal to the distally extending hooks.

In some examples, the valve assembly further comprises three proximally extending angled spikes, each extending at a sharp angle β radially outward and in the proximal direction from a corresponding engagement frame inflow apex.

In some examples, each proximally extending angled spike terminates with a sharp distal tip.

In some examples, the angle β is in the range of 10-80 degrees.

In some examples, the angle β is in the range of 20-70 degrees.

In some examples, the angle β is in the range of 30-60 degrees.

According to another aspect of the disclosure, there is provided a method comprising positioning a valve assembly between native or host leaflets within a patient's body, wherein the valve assembly comprises prosthetic valve and a leaflet engagement frame. The prosthetic valve comprises a valve frame movable between a radially compressed state and a radially expanded state, and a leaflet assembly mounted within the valve frame. The valve frame is defined between a valve outflow end and a valve inflow end, and comprises intersecting struts that define at least one row of circumferentially extending frame cells. The leaflet assembly comprises a plurality of leaflets configured to regulate flow through the prosthetic valve.

The leaflet engagement frame which is disposed radially outwardly from, and is coupled to, the valve frame. The leaflet engagement frame comprises first struts defined along an engagement frame first rung, and second struts defined along an engagement frame second rung, distal to the engagement frame first rung. The leaflet engagement frame further comprises three engagement cells, each engagement cell defined by two first struts extending from an engagement frame outflow apex to engagement frame intermediate junctions, and two second struts extending from the two engagement frame intermediate junctions to an engagement frame inflow apex.

The leaflet engagement frame further comprises three distally extending hooks, wherein each distally extending hook comprises a curved portion connected on one end to a corresponding engagement frame outflow apex, and an opposite free end positioned radially away from the corresponding outflow apex and oriented in a distal direction.

The method further comprises partially expanding the valve assembly at least until the distally extending hooks capture proximal ends of native or host leaflets within the curved portions.

The method further comprises further expanding the valve assembly such that the distally extending hooks push against the native or host leaflets in a distal direction to a folded configuration of the native or host leaflets.

In some examples, the valve frame comprises a row with more than three cells. In some examples, the valve frame comprises a row with six cells. In some examples, the valve frame comprises a row with nine cells. In some examples, the valve frame comprises a row with twelve cells. Any example of a row of the valve frame comprising more than three cells, refers to a row underlying at least a portion of the leaflet engagement frame.

In some examples, the engagement frame outflow apices are distal to the valve outflow end.

In some examples, the first struts and the second struts of the leaflet engagement frame are not aligned with any of the struts of the valve frame.

In some examples, the second struts are longer than the first struts.

In some examples, the method further comprises a step of advancing the valve assembly in a distal direction after the step of partially expanding the valve assembly, such that the distally extending hooks push against the native or host leaflets, so as to displace their proximal ends in a distal direction along with the valve assembly during its advancement in the partially expanded state.

In some examples, the leaflet engagement frame further comprises three proximally extending angled spikes, each extending at a sharp angle β radially outward and in the proximal direction from a corresponding engagement frame inflow apex.

In some examples, each proximally extending angled spike terminates with a sharp distal tip.

In some examples, the angle β is in the range of 10-80 degrees.

In some examples, the angle β is in the range of 20-70 degrees.

In some examples, the angle β is in the range of 30-60 degrees.

According to another aspect of the disclosure, there is provided a valve assembly comprising a prosthetic valve and a leaflet engagement frame. The prosthetic valve comprises a valve frame movable between a radially compressed state and a radially expanded state, a leaflet assembly mounted within the valve frame, and an outer skirt attached to the valve frame. The leaflet assembly comprises a plurality of leaflets configured to regulate flow through the prosthetic valve. The outer skirt comprises a floating fiber portion disposed between an outer skirt outflow end and an outer skirt inflow end. The floating fiber portion comprises a plurality of filaments extending radially outward away from the valve frame in the radially expanded state. The leaflet engagement frame is coupled to the outer skirt and comprises at least one rung of engagement frame struts, and a plurality of spikes extending from the engagement frame struts. The spikes are configured to engage with the native tissue outside the valve assembly to irritate the native tissue.

According to another aspect of the disclosure, there is provided a valve assembly comprising a prosthetic valve and a leaflet engagement frame. The prosthetic valve comprises a valve frame movable between a radially compressed state and a radially expanded state, a leaflet assembly mounted within the valve frame, and an outer skirt attached to the valve frame. The leaflet assembly comprises a plurality of leaflets configured to regulate flow through the prosthetic valve. The outer skirt comprises sealing ring extending radially away from the valve frame. The leaflet engagement frame is disposed radially outwardly from, and is coupled to, the valve frame. The leaflet engagement frame comprises at least one rung of engagement frame struts, and a plurality of spikes extending from the engagement frame struts. The spikes are configured to contact native tissue to help secure the valve assembly at an implantation location.

According to another aspect of the disclosure, there is provided a valve assembly comprising a prosthetic valve and a restriction frame. The prosthetic valve comprises a valve frame configured to self-expand from a first diameter in a radially compressed state to a third diameter in a free radially expanded state, and a leaflet assembly mounted within the valve frame. The leaflet assembly comprises a plurality of leaflets configured to regulate flow through the prosthetic valve. The restriction belt is disposed radially outwardly from, and is coupled to, the valve frame. The restriction frame comprises at least one rung of intersecting restriction struts. The restriction frame is configured to limit the valve frame to a second diameter in a deployed state thereof, wherein the second diameter is less than the third diameter.

Certain examples of the disclosed technology may include some, all, or none of the above advantages. Further advantages may be readily apparent to those skilled in the art from the figures, descriptions, and claims included herein. Aspects and examples of the disclosed technology are further described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following examples and aspects thereof are described and illustrated in conjunction with systems, tools and methods which are meant to be exemplary and illustrative, but not limiting in scope. In various examples, one or more of the above-described problems have been reduced or eliminated, while other examples are directed to other advantages or improvements.

### BRIEF DESCRIPTION OF THE FIGURES

Some examples of the disclosure are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the disclosure. For the sake of clarity, some objects depicted in the figures are not to scale.

In the Figures:

FIG. 1A shows a perspective view of a prosthetic valve, according to some examples.

FIG. 1B shows a perspective view of a frame of the prosthetic valve of FIG. 1A.

FIG. 2 shows a perspective view of a delivery assembly comprising a delivery apparatus carrying a balloon expandable prosthetic device, according to some examples.

FIG. 3 shows a cross sectional view of a native aortic valve.

FIG. 4A shows a side view of a prosthetic valve implanted in the native aortic valve.

FIG. 4B shows the implanted prosthetic valve of FIG. 2A as viewed from the ascending aorta.

FIG. 5 shows a perspective view of a leaflet anchoring frame with distally extending angles spikes, according to some examples.

FIG. 6A shows a side elevation view of the valve frame of FIG. 1B, depicting the valve frame in a flat configuration.

FIG. 6B shows a side elevation view of the leaflet anchoring frame of FIG. 5, depicting the leaflet anchoring frame in a flat configuration.

FIG. 7 shows a perspective view of an exemplary valve assembly comprising the prosthetic valve of FIGS. 1A-B and the leaflet anchoring frame of FIG. 5.

FIG. 8A shows a detail view of the valve assembly of FIG. 7.

FIG. 8B shows a cross-sectional view along line 8B-8B of FIG. 8A.

FIGS. 9A-9D show stages of a method for implanting the valve assembly of FIG. 7, according to some examples.

FIG. 10 shows a perspective view of another examples of a valve assembly.

FIG. 11 shows a side elevation view of a leaflet anchoring frame comprising two rungs of struts, depicting the leaflet anchoring frame in a flat configuration, according to some examples.

FIG. 12 shows a perspective view of an example of a valve assembly comprising the leaflet anchoring frame of FIG. 11.

FIG. 13 shows a side elevation view of a leaflet anchoring frame comprising a single rung of struts, depicting the leaflet anchoring frame in a flat configuration, according to some examples.

FIG. 14 shows a perspective view of an example of a valve assembly comprising the leaflet anchoring frame of FIG. 13.

FIGS. 15A-15D show stages of a method for implanting the valve assembly of FIG. 14, according to some examples.

FIGS. 16A-16C show different examples of prosthetic valve with integral distally extending angled spikes.

FIG. 17A shows a perspective view of a mechanically expandable prosthetic valve, according to some examples.

FIG. 17B shows a perspective view of a frame of the prosthetic valve of FIG. 17A.

FIG. 18 shows a perspective view of a delivery assembly comprising a delivery apparatus for use with the prosthetic valve of FIGS. 17A-B, according to some examples.

FIG. 19 shows a perspective view of another example of a leaflet anchoring frame.

FIG. 20 shows a perspective view of another example of a valve assembly comprising the prosthetic valve of FIGS. 17A-B and the leaflet anchoring frame of FIG. 19.

FIGS. 21A-21C show stages of a method for implanting the valve assembly of FIG. 20, according to some examples.

FIG. 22 shows a perspective view of another example of a leaflet anchoring frame, comprising a single rung of curved struts.

FIG. 23 shows a perspective view of a valve assembly comprising the leaflet anchoring frame of FIG. 22.

FIG. 24 shows a perspective view of a valve assembly comprising leaflet engagement wire wrapped around struts of a prosthetic valve, according to some examples.

FIG. 25 shows a perspective view of a prosthetic valve with integral distally extending angled spikes, according to some examples.

FIG. 26 shows a perspective view of a leaflet anchoring frame with distally extending hooks, according to some examples.

FIG. 27 shows a perspective view of an exemplary valve assembly comprising a leaflet anchoring frame with distally extending hooks.

FIGS. 28A-28B show stages of a method for implanting the valve assembly of FIG. 27, according to some examples.

FIG. 29 shows a perspective view of another exemplary valve assembly comprising a leaflet anchoring frame with distally extending hooks.

FIG. 30 shows a side view of a prosthetic valve comprising an outer skirt equipped with a floating fiber portion.

FIG. 31A shows a perspective view of another exemplary valve assembly comprising a leaflet engagement frame coupled to the prosthetic valve of FIG. 30.

FIG. 31B shows a cross-sectional view along line 31B-31B of FIG. 31A.

FIG. 32 shows a perspective view of another exemplary valve assembly comprising a leaflet engagement frame coupled to a prosthetic valve equipped with a sealing ring.

FIG. 33 shows the valve assembly of FIG. 32 disposed within a native mitral valve.

FIG. 34A shows a perspective view of an exemplary valve assembly comprising a restriction frame coupled to a prosthetic valve.

FIG. 34B shows a cross-sectional view along line 34B-34B of FIG. 34A.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

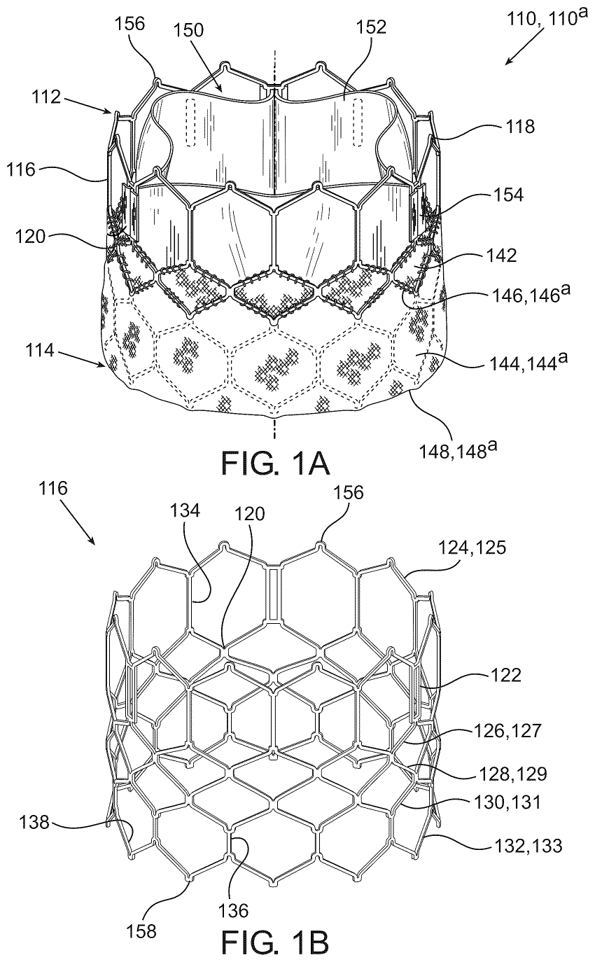

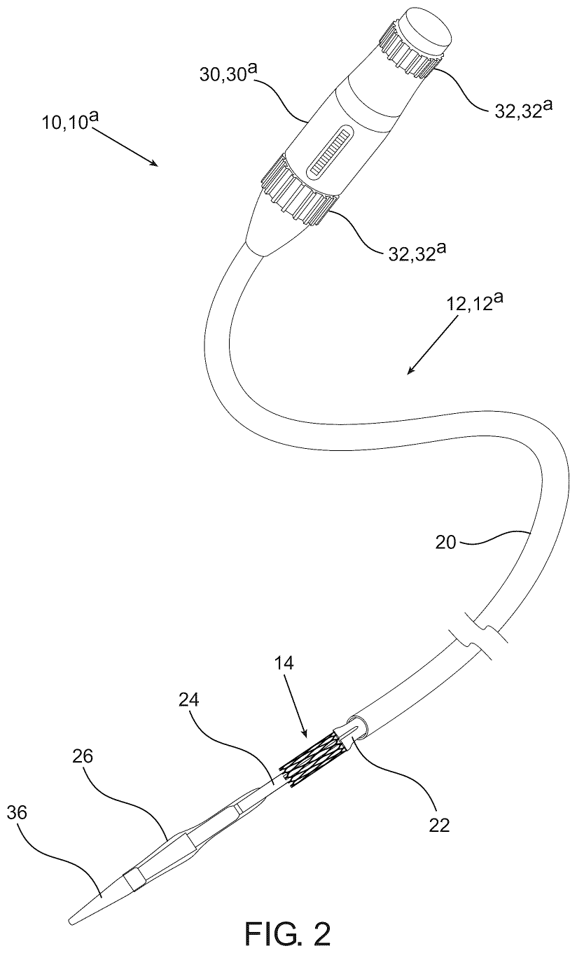

FIGS. 1A and 1B show perspective views of a prosthetic valve 110 with and without soft components attached thereto, according to some examples. FIG. 2 shows a perspective view of a delivery assembly 10, according to some examples. The delivery assembly 10 can include the prosthetic valve 110 and a delivery apparatus 12. The prosthetic valve 110 can be on or releasably coupled to the delivery apparatus 12. The delivery apparatus can include a handle 30 at a proximal end thereof, and a nosecone shaft extending distally from the handle 30, having a nosecone 36 attached to its distal end.

The term “proximal”, as used herein, generally refers to the side or end of any device or a component of a device, which is closer to the handle 30 or an operator of the handle 30 when in use.

The term “distal”, as used herein, generally refers to the side or end of any device or a component of a device, which is farther from the handle 30 or an operator of the handle 30 when in use.

The term “prosthetic valve”, as used herein, refers to any type of a prosthetic valve deliverable to a patient's target site over a catheter, which is radially expandable and compressible between a radially compressed, or crimped, state, and a radially expanded state. Thus, a prosthetic valve 110 can be crimped or retained by a delivery apparatus 12 in a compressed state during delivery, and then expanded to the expanded state once the prosthetic valve 110 reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

The term “plurality”, as used herein, means more than one.

A prosthetic valve 110 of the current disclosure may include any prosthetic valve configured to be mounted within the native aortic valve, the native mitral valve, the native pulmonary valve, and the native tricuspid valve. While a delivery assembly 10 described in the current disclosure, includes a delivery apparatus 12 and a balloon expandable prosthetic device 14, such as prosthetic valve 110 or prosthetic assembly 100, it should be understood that the delivery apparatus 12 according to any example of the current disclosure can be used for implantation of other prosthetic devices aside from prosthetic valves, such as stents or grafts.

A catheter deliverable prosthetic valve 110 can be delivered to the site of implantation via the delivery assembly 10 carrying the valve 110 in a radially compressed or crimped state, toward the target site, to be mounted against the native anatomy, by expanding the prosthetic valve 110 via various expansion mechanisms. Balloon expandable valves generally involve a procedure of inflating a balloon within a prosthetic valve, thereby expanding the prosthetic valve 110 within the desired implantation site. Once the valve is sufficiently expanded, the balloon is deflated and retrieved along with the delivery apparatus 12. Self-expandable valves include a frame that is shape-set to automatically expand as soon an outer retaining capsule, which may be also defined as the distal portion of an outer shaft (20) or the distal portion of a delivery shaft, is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies, releasably coupled to respective actuation assemblies of the delivery apparatus 12, controlled via the handle 30 for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus 12 once the prosthetic valve is properly positioned at the desired site of implantation.

The delivery assembly 10 can be utilized, for example, to deliver a prosthetic aortic valve for mounting against the aortic annulus, to deliver a prosthetic mitral valve for mounting against the mitral annulus, or to deliver a prosthetic valve for mounting against any other native annulus.

An exemplary delivery assembly 10^{a} illustrated in FIG. 2 can include a delivery apparatus 12^{a} for delivery and implantation of a balloon expandable prosthetic device 14, such as a balloon expandable prosthetic valve that can be similar to the prosthetic valve 110 illustrated in FIGS. 1A-B, as well as other prosthetic valves 210 (e.g., prosthetic valve 210^{a}, 210^{b}, 210^{c}) or valve assemblies 100 (e.g., valve assembly 100^{a}, 100^{b}, 100^{c}) that will described in greater detail further below. According to some examples, the delivery apparatus 12^{a} includes a handle 30^{a} and a balloon catheter 24 having an inflatable balloon 26 mounted on its distal end. The balloon expandable prosthetic device 14 can be carried in a crimped state over the balloon catheter 24. Optionally, an outer shaft 20 can concentrically extend over the balloon catheter 24, and a push shaft 22 disposed over the balloon catheter 24, optionally between the balloon catheter 24 and the outer shaft 20.

The outer shaft 20, the push shaft 22, and the balloon catheter 24, can be configured to be axially movable relative to each other. For example, a proximally oriented movement of the outer shaft 20 relative to the balloon catheter 24, or a distally oriented movement of the balloon catheter 24 relative to the outer shaft 20, can expose the prosthetic device 14 from the outer shaft 20. The delivery apparatus 12^{a} can further include a nosecone 36 connected to the distal end of a nosecone shaft (hidden from view in FIG. 2) extending through a lumen of the balloon catheter 24.

Outer shaft 20, push shaft 22, balloon 26, balloon catheter 24, and nosecone 36 along with the nosecone shaft can be formed from any of various suitable materials, such as nylon, braided stainless steel wires, or a polyether block amide (commercially available as Pebax®). In one example, outer shaft 20 and push shaft 22 have longitudinal sections formed from different materials in order to vary the flexibility of the shafts along their lengths. In another example, the nosecone shaft has an inner liner or layer formed of Teflon® to minimize sliding friction with a guide wire (not shown).

The proximal ends of the balloon catheter 24, the outer shaft 20, the push shaft 22, and optionally the nosecone shaft, can be coupled to the handle 30^{a}. During delivery of the prosthetic device 14, the handle 30^{a} can be maneuvered by an operator (e.g., a clinician or a surgeon) to axially advance or retract components of the delivery apparatus 12^{a}, such as the nosecone shaft, the balloon catheter 24, the outer shaft 20, and/or the push shaft 22, through the patient's vasculature, as well as to inflate the balloon 26 mounted on the balloon catheter 24, so as to expand the prosthetic device 14, and to deflate the balloon 26 and retract the delivery apparatus 12^{a} once the prosthetic device 14 is mounted in the implantation site.

The handle 30^{a} can include a steering mechanism configured to adjust the curvature of the distal end portion of the delivery apparatus 12^{a}. In the illustrated example, the handle 30^{a} includes an adjustment member, such as one of the illustrated rotatable knobs 32^{a}, which in turn is operatively coupled to the proximal end portion of a pull wire. The pull wire can extend distally from the handle 30^{a} through the outer shaft 20 and has a distal end portion affixed to the outer shaft 20 at or near the distal end of the outer shaft 20. Rotating the knob 32^{a} can increase or decrease the tension in the pull wire, thereby adjusting the curvature of the distal end portion of the delivery apparatus 12^{a}. Further details on steering or flex mechanisms for the delivery apparatus can be found in U.S. Pat. No. 9,339,384, which is incorporated by reference herein. The handle 30^{a} can further include an adjustment mechanism including an adjustment member, such as the other illustrated rotatable knob 32^{a}. The adjustment mechanism can be configured to adjust the axial position of the push shaft 620 relative to the balloon catheter.

The prosthetic device 14 can be carried by the delivery apparatus 12^{a} during delivery in a crimped state, and expanded by balloon inflation to secure it in a native heart valve annulus. In one exemplary implantation procedure, the prosthetic device 14 is initially crimped over the balloon catheter 24, proximal to the inflatable balloon 26. Because prosthetic device 14 is crimped at a location different from the location of balloon 26, prosthetic device 14 can be crimped to a lower profile than would be possible if it was crimped on top of balloon 26. This lower profile permits the clinician to more easily navigate the delivery apparatus 12^{a} (including crimped prosthetic valve 110 or 210, or crimped valve assembly 100) through a patient's vasculature to the treatment location. The lower profile of the crimped prosthetic device is particularly helpful when navigating through portions of the patient's vasculature which are particularly narrow, such as the iliac artery.

The balloon 26 can be secured to balloon catheter 24 at its balloon proximal end, and to either the balloon catheter 24 or the nosecone 36 at its distal end. The distal end portion of the push shaft 22 is positioned proximal to the outflow end (e.g., valve outflow end 112 or 212, which will be described further below) of the prosthetic device 14.

When reaching the site of implantation, and prior to balloon inflation, the push shaft 22 is advanced distally, allowing its distal end portion to contact and push against the outflow end of prosthetic device 14, pushing the device 14 distally therewith. The distal end of push shaft 22 is dimensioned to engage with the outflow end of the prosthetic device 14 in a crimped configuration of the device. In some implementations, the distal end portion of the push shaft 22 can be flared radially outward, to terminate at a wider-diameter that can contact the prosthetic device 14 in its crimped state. Push shaft 22 can then be advanced distally, pushing the prosthetic device 14 therewith, until the crimped prosthetic device 14 is disposed around the balloon 26, at which point the balloon 26 can be inflated to radially expand the prosthetic device 14. Once the prosthetic device 14 is expanded to its functional diameter within a native annulus, the balloon 26 can be deflated, and the delivery apparatus 12^{a} can be retrieved from the patient's body.

In particular implementations, the delivery apparatus 12 (including any of delivery apparatus 12^{a} or 12^{b} described further below) with the prosthetic device (including any of prosthetic valves 110, 210, 510 and any of valve assemblies 100, 300, 400 and 600 described in greater detail throughout the specification) assembled thereon, can be packaged in a sterile package that can be supplied to end users for storage and eventual use. In particular implementations, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic device (e.g., any of balloon expandable prosthetic valve 110, 210, balloon expandable valve assembly 100, mechanically expandable prosthetic valve 310, 510, and mechanically expandable valve assembly 300, 400 and 600, which will be described in greater detail throughout the specification) and the delivery apparatus 12 can be free of any liquid. Methods for treating tissue leaflets for dry storage are disclosed in U.S. Pat. Nos. 8,007,992 and 8,357,387, both of which documents are incorporated herein by reference.

FIGS. 1A-B show an example of a prosthetic valve 110, which can be a balloon expandable valve, illustrated in an expanded state. The prosthetic valve 110 can comprise a valve outflow end 112 and a valve inflow end 114. In some instances, the valve outflow end 112 is the proximal end of the prosthetic valve 110, and the valve inflow end 114 is the distal end of the prosthetic valve 110. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the valve 110.

The term “inflow”, as used herein, refers to a region of the prosthetic valve through which the blood flows into the valve 110.

The valve 110 comprises an annular valve frame 116 movable between a radially compressed configuration and a radially expanded configuration, and a leaflet assembly 150 mounted within the valve frame 116. The valve frame 116 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the valve frame 116 can be crimped to a radially compressed state on a balloon catheter 24, and then expanded inside a patient by an inflatable balloon or equivalent expansion mechanism. Alternatively or additionally, the valve frame 116 can be made of shape-memory materials such as, but not limited to, nickel titanium alloy (e.g., Nitinol). When constructed of a shape-memory material, the valve frame 116 can be crimped to a radially compressed state and restrained in the compressed state by insertion into a shaft or equivalent mechanism of a delivery apparatus 12.

In the example illustrated in FIGS. 1A-B, the valve frame 116 is an annular, stent-like structure comprising a plurality of intersecting struts 118. In this application, the term “strut” encompasses axial struts, angled struts, support posts, commissure windows, and any similar structures described by U.S. Pat. Nos. 7,993,394 and 9,393,110, which are incorporated herein by reference. A strut 118 may be any elongated member or portion of the valve frame 116. The valve frame 116 can include a plurality of strut rungs that can collectively define one or more rows of cells 138. The valve frame 116 can have a cylindrical or substantially cylindrical shape having a constant diameter from the inflow end 114 to the outflow end 112 as shown, or the frame can vary in diameter along the height of the frame, as disclosed in U.S. Pat. No. 9,155,619, which is incorporated herein by reference.

The end portions of the struts 118 are forming apices 156 at the valve outflow end 112 and apices 158 at the valve inflow end 114. The struts 118 can intersect at additional valve frame junctions 120 formed between the outflow apices 156 and the inflow apices 158. The valve frame junctions 120 can be equally or unequally spaced apart from each other, and/or from the apices 156, 158, between the outflow end 112 and the inflow end 114.

According to some examples, the valve frame struts 118 include a plurality of angled struts and vertical or axial struts. FIGS. 1A-B show an example of a type of a prosthetic valve 110, that can be representative of, but is not limited to, a balloon expandable prosthetic valve. The valve frame 116 of the prosthetic valve 110 illustrated in FIG. 1B comprises rungs of angled struts 125, 127, 129, 131, 133, and axial struts 134, 136. In such examples, the struts may be pivotable or bendable relative to each other, so as to permit frame expansion or compression. For example, the valve frame 116 can be formed from a single piece of material, such as a metal tube, via various processes such as, but not limited to, laser cutting, electroforming, and/or physical vapor deposition, while retaining the ability to collapse/expand radially in the absence of hinges and like.

The leaflet assembly 150 comprises a plurality of leaflets 152 (e.g., three leaflets), positioned at least partially within the valve frame 116, and configured to regulate flow of blood through the prosthetic valve 110 from the inflow end 114 to the outflow end 112. While three leaflets 152 arranged to collapse in a tricuspid arrangement, are shown in the example illustrated in FIG. 1A, it will be clear that a prosthetic valve 110 can include any other number of leaflets 152. Adjacent leaflets 152 can be arranged together to form commissures 154 that are coupled (directly or indirectly) to respective portions of the valve frame 116, thereby securing at least a portion of the leaflet assembly 150 to the frame 116. The leaflets 152 can be made from, in whole or part, biological material (e.g., pericardium), bio-compatible synthetic materials, or other such materials. Further details regarding transcatheter prosthetic heart valves, including the manner in which the leaflet assemblies 150 can be coupled to the frame 116 of the prosthetic valve 110, can be found, for example, in U.S. Pat. Nos. 6,730,118, 7,393,360, 7,510,575, 7,993,394, and 8,652,202, and U.S. Patent Application Publication No. 2018/0325665, all of which are incorporated herein by reference in their entireties.

According to some examples, the prosthetic valve 110 can further comprise at least one skirt or sealing member. An inner skirt 142 can be secured to the inner surface of the valve frame 116, configured to function, for example, as a sealing member to prevent or decrease perivalvular leakage. An inner skirt 142 can further function as an anchoring region for the leaflets 152 to the valve frame 116, and/or function to protect the leaflets 152 against damage which may be caused by contact with the valve frame 116, for example during valve crimping or during working cycles of the prosthetic valve 110. Additionally, or alternatively, the prosthetic valve 110 can comprise an outer skirt 144 mounted on the outer surface of the valve frame 116, configure to function, for example, as a sealing member retained between the valve frame 116 and the surrounding tissue of the native annulus against which the prosthetic valve is mounted, thereby reducing risk of paravalvular leakage (PVL) past the prosthetic valve 110.

Any of the inner skirt 142 and/or outer skirt 144 can be made of various suitable biocompatible materials, such as, but not limited to, various synthetic materials (e.g., PET) or natural tissue (e.g. pericardial tissue). In some examples, the inner skirt 142 comprises a single sheet of material that extends continuously around the inner surface of the valve frame 116. In some examples, the outer skirt 144 comprises a single sheet of material that extends continuously around the outer surface of the valve frame 116, as shown for outer skirt 144^{a} of prosthetic valve 110^{a} illustrated in FIG. 1A.

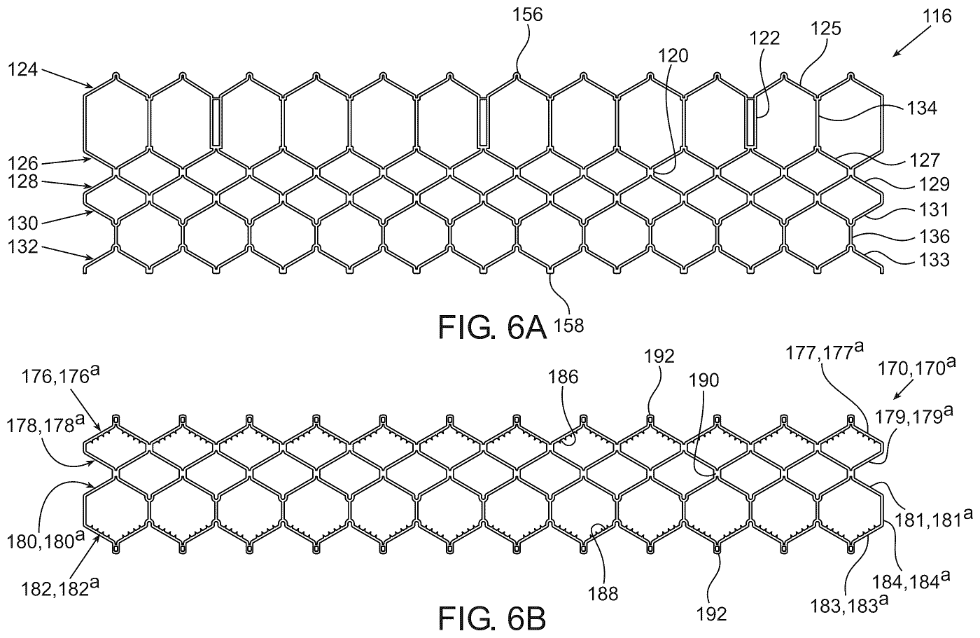

FIGS. 1B and 6A show the valve frame 116 of the prosthetic valve 110 with the other components, such as leaflets and skirts, removed. While FIG. 1B shows the valve frame 116 in an annular configuration, corresponding to its functional configuration, FIG. 6A shows the valve frame 116 in a flat configuration for purposes of illustration. The valve frame 116 can comprise, in some examples, a plurality of rows or rungs of angled struts, including a valve frame outflow rung 124, a valve frame inflow rung 132, and a plurality of valve frame intermediate rungs 126, 128, 130.

In the examples illustrated in FIGS. 1B and 6A, the valve frame 116 comprises a valve frame outflow rung 124 of angled struts 125 arranged end-to-end and extending circumferentially at the valve outflow end 112; a valve frame first intermediate rung 126 of circumferentially extending, angled struts 127; a valve frame second intermediate rung 128 of circumferentially extending, angled struts 129; a valve frame third intermediate rung 130 of circumferentially extending, angled struts 131; and a valve frame inflow rung 132 of angled struts 133 extending circumferentially at the valve inflow end 114.

A plurality of substantially straight proximal axial struts 134 can be used to interconnect the angled struts 125 of the valve frame outflow rung 124 with the angled struts 127 of the valve frame first intermediate rung 126. Specifically, each valve frame proximal axial strut 134 extends from a location defined by the convergence of upper ends of two angled struts 127 to another location defined by the convergence of lower ends of two angled struts 125.

Similarly, a plurality of substantially straight distal axial struts 136 can be used to interconnect the angled struts 133 of the valve frame inflow rung 132 with the angled struts 131 of the valve frame third intermediate rung 130. Specifically, each valve frame distal axial strut 136 extends from a location defined by the convergence of upper ends of two angled struts 133 to another location defined by the convergence of lower ends of two angled struts 131.

The axial length of the valve frame proximal axial struts 134 can be different than that of the valve frame distal axial struts 136. For example, in the illustrated implementation the valve frame proximal axial struts 134 can be longer than the valve frame distal axial struts 136. In some examples, at least some (e.g., three) of the valve frame proximal axial struts 134 can define axially extending window frame portions, also termed commissure windows 122, configured to mount respective commissures 154 of the leaflet assembly 150.

The struts collectively define a plurality of cells 138 of the valve frame 116. At the inflow end of the frame 116, angled struts 133 of the valve frame inflow rung 132, valve frame distal axial struts 136, and angled struts 131 of the valve frame third intermediate rung 130, define a lower row of cells 138. The angled struts 131, 129 and 127 of the valve frame third, second and first intermediate rungs 130, 128 and 126, respectively, define two intermediate rows of cells 138. The angled struts 125 of the valve frame outflow rung 124, valve frame proximal axial struts 134, and angled struts 127 of the valve frame first intermediate rung 126, define an upper row of cells 138. The relatively greater length of the valve frame proximal axial struts 134 results in relatively larger openings defined by the upper cells 138.

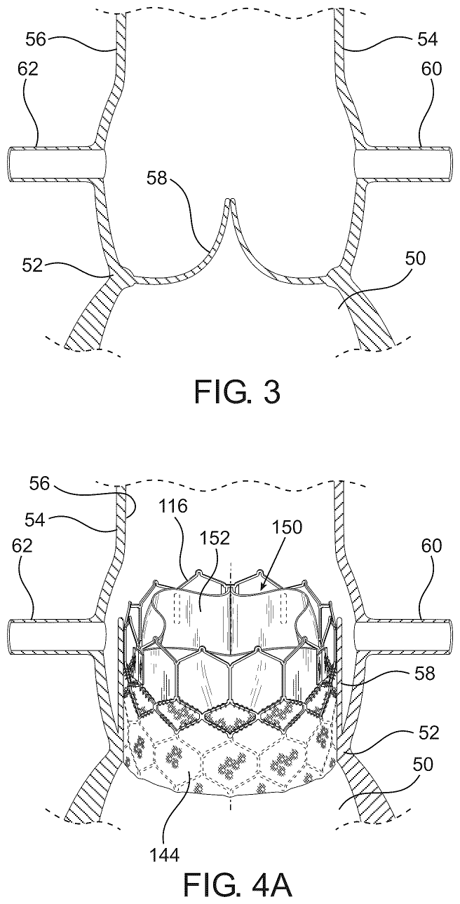

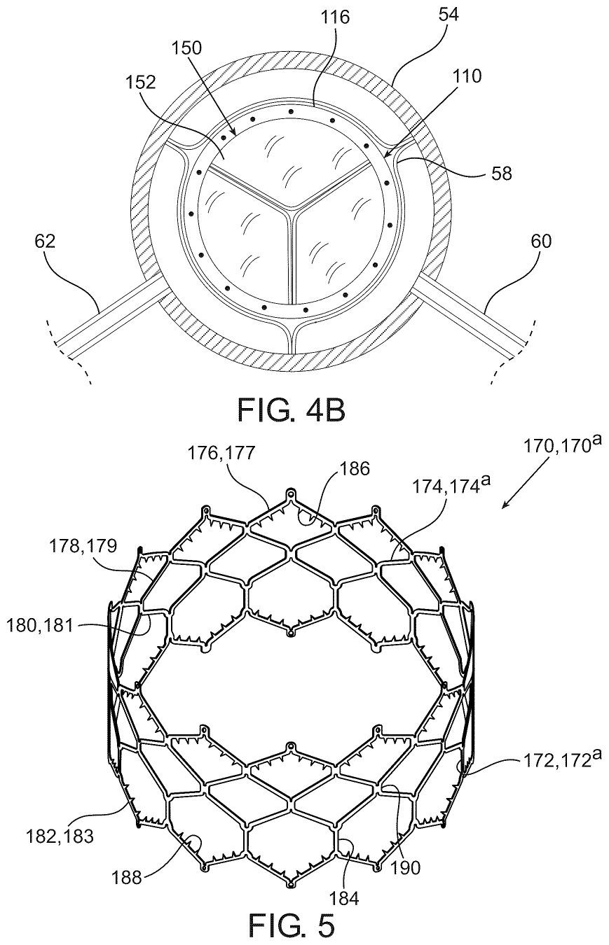

FIG. 3 shows the anatomy of the aortic root of a native valve, which has a plurality of native leaflets 58 (e.g., three leaflets, although only two are illustrated in the simplified illustration of FIG. 3) separating the left ventricle 50 from the ascending aorta 54. FIGS. 4A-B show the exemplary prosthetic valve 110 implanted within the aortic annulus 52 of the native valve. The prosthetic valve 110 may be deployed in between the native or host leaflets such that the native or host leaflets are pushed radially outward upon expansion of the prosthetic valve 110. Such a configuration may result whether the prosthetic valve 110 is deployed within a native valve, as illustrated in FIGS. 4A-B, or within a host prosthetic valve that has been previously deployed.

The outward radial push of the native or host leaflets may cause a variety of maladies. For example, FIG. 4A schematically shows the native leaflets 58 extending proximally, or in the outflow direction, from the aortic annulus 52. The leaflets 58 are positioned between a flow channel of the native aortic valve and the aortic wall 56. Notably, coronary ostia may be positioned on the surface of the aorta. As the prosthetic valve is expanded within the native aortic valve, there is a possibility that the expanded prosthetic valve may radially push the native leaflets 58 outward such that the native leaflets 58 may obstruct the ostia of the coronary arteries 60, 62, which may lead to coronary maladies. A similar concern may result if a prosthetic valve were expanded within a previously expanded or host prosthetic valve within the aortic annulus 52.

To avoid obstruction of blood flow to the coronary arteries 60, 62, the native or host leaflets (i.e., leaflets of a native aortic valve or of a previously implanted prosthetic valve) can be displaced and/or folded during implantation of a new prosthetic valve within the existing valvular structure.

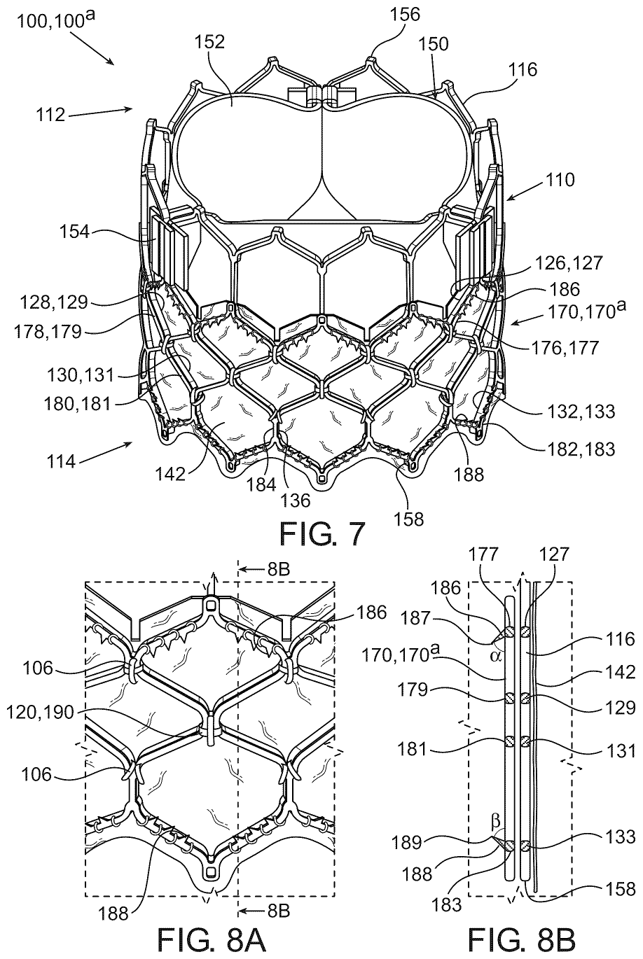

In some examples, a valve assembly 100 is disclosed. The valve assembly 100 (shown, for example, in FIGS. 7-8B) comprises the prosthetic valve 110 and a leaflet engagement frame 170 (shown, for example, in FIG. 5) which is disposed radially outwardly from, and is coupled to, the valve frame 116. The leaflet engagement frame 170 comprises leaflet engaging features, such as spikes, designed to engage and position one, some, or all of the native or host leaflets away from a level of the coronary arteries during the procedure of valve assembly 100 implantation, thereby maintaining vascular access to the coronary arteries. The leaflet engagement frame 170 comprises at least one rung of struts, referred to as the engagement frame first rung 176, which can be, in some implementations, the single rung provided with a plurality of distally extending angled spikes 186. In some examples, the leaflet engagement frame 170 further comprises at least one additional rung of struts, distal to the engagement frame first rung 176 (and to the distally extending angled spikes 186), which can be, in some implementations, the single rung provided with proximally extending angled spikes 188, opposite to the distally extending angled spikes 186.

The leaflet engagement frame 170 comprises a plurality of engagement frame struts 174 configured in an annular shape. The engagement frame struts 174 define at least one rung, which can be referred to as an engagement frame first rung 176 defined by a plurality of first angled struts 177 at the proximal or outflow end of the leaflet engagement frame 170. The first angled struts 177 can be, in some implementations, the sole struts that comprise distally extending angled spikes 186, extending radially outward and in the distal direction (i.e., downward or toward the inflow end) therefrom.

Stated otherwise, a sharp angle α (shown for example in FIG. 8B) is defined between an annular surface 175 defined by the first angled struts 177 of the engagement frame first rung 176, and each distally extending angled spike 186. The angle α can be similarly defined between each distally extending angled spike 186 and a central longitudinal axis extending from the inflow end 114 to the outflow end 112, meaning that the distally extending angled spikes 186 are not parallel to the central longitudinal axis or any axis that extends in parallel to the central longitudinal axis. In some examples, the angle α is in the range of 10-80 degrees. In some examples, the angle α is in the range of 20-70 degrees. In some examples, the angle α is in the range of 30-60 degrees.

The distally extending angled spikes 186 are configured to engage (and in some instances penetrate) the native or host leaflets when the valve assembly 100 is partially expanded, and remain engaged with the leaflets as the valve assembly 100 is further expanded to its final expansion diameter. In some examples, each distally extending angled spike 186 terminates with a sharp distal tip 187.

FIGS. 5 and 6B show an example of a leaflet engagement frame 170^{a}. FIG. 5 shows the leaflet engaging frame 170^{a} in an annular configuration, detached from the prosthetic valve 110, and FIG. 6B shows the leaflet engagement frame 170^{a} in a flat configuration for purposes of illustration. In some examples, the leaflet engagement frame 170 comprises a plurality of rungs of angled struts, such as four rungs shown for the example of leaflet engagement frame 170^{a}.

Leaflet engagement frame 170^{a} comprises an engagement frame first rung 176^{a} of circumferentially extending, first angled struts 177^{a}; an engagement frame second rung 178^{a} of circumferentially extending, second angled struts 179^{a}; an engagement frame third rung 180^{a} of circumferentially extending, third angled struts 181^{a}; and an engagement frame fourth rung 182^{a} of circumferentially extending, fourth angled struts 183^{a}. A plurality of substantially straight axial struts 184^{a} can be used to interconnect the fourth angled struts 183^{a} of the engagement frame fourth rung 182^{a} with the third angled struts 181^{a} of the engagement frame third rung 180^{a}. Specifically, each engagement frame axial strut 184^{a} extends from a location defined by the convergence of upper ends of two fourth angled struts 183^{a} to another location defined by the convergence of lower ends of two third angled struts 181^{a}.

The engagement frame struts collectively define a plurality of engagement frame cells 172^{a}. At the inflow end of the engagement frame 170^{a}, fourth angled struts 183^{a} of the engagement frame fourth rung 182^{a}, engagement frame axial struts 184^{a}, and third angled struts 181^{a} of the engagement frame third rung 180^{a}, define a lower row of cells 172^{a}. The angled struts 181^{a}, 179^{a} and 177^{a} of the engagement frame third, second and first rungs 180^{a}, 178^{a} and 176^{a}, respectively, define the two upper rows of cells 172^{a}.

FIG. 7 shows the leaflet engagement frame 170^{a} mounted over the prosthetic valve 110, together forming the valve assembly 100^{a}. The leaflet engagement frame 170^{a} is configured such that the angled struts of the engagement frame first rung 176^{a}, second rung 178^{a}, third rung 180^{a} and fourth rung 182^{a}, align with the angled struts of the valve frame first intermediate rung 126, second intermediate rung 128, third intermediate rung 130, and inflow rung 132, respectively. Similarly, the engagement frame axial struts 184^{a} align with the valve frame distal axial struts 136, and the three rows of engagement frame cells 172^{a} align with the corresponding lower three rows of cells 138 of the valve frame 116.

In this manner, engagement frame junctions 190 can be coupled to valve frame junctions 120, as shown in FIG. 8A. The term “valve frame junctions 120” refers to any of the outflow apices 156, inflow apices 158, or any other non-apical junctions defined therebetween by intersection struts 118 of the valve frame 116. Similarly, the term “engagement frame junctions 190” refers to any of the inflow apices, outflow apices, or any other non-apical junctions defined therebetween by intersecting struts 174 of the leaflet engagement frame 170.

In the example illustrated in FIG. 8A, the leaflet engagement frame 170 is coupled to the valve frame 116 with sutures 106. In some examples, the leaflet engagement frame 170 comprises engagements frame junction apertures 192, that can be formed as openings formed in any of the engagement frame junctions 190, configured to receive the sutures 106. It should be noted that, for purpose of illustration, the sutures 106 are shown only in FIG. 8A and not in other figures of the valve assemblies. In other examples, the leaflet engagement frame 170 can be coupled to the valve frame 116 in various other ways (e.g., fasteners, welding, adhesive, etc.). By coupling the engagement frame junctions 190 to the valve frame junctions 120, the leaflet engagement frame 170 can, for example, expand and/or contract simultaneously with the valve frame 116.

In some examples, the leaflet engagement frame 170 is removably coupled to the valve frame 116 (e.g., with sutures 106 and/or fasteners). The term “removably coupled”, as used herein, means coupled in such a way that two components are coupled together and can be separated without plastically deforming either of the components. In other examples, the leaflet engagement frame 170 can be permanently coupled in such a way that the two components cannot be separated without plastically deforming at least one of the components.

The leaflet engagement frame can be made of any of various suitable plastically-deformable materials (e.g., stainless steel, etc.) and/or self-expanding materials (e.g., Nitinol). When the leaflet engagement frame comprises plastically-expandable material, the leaflet engagement frame (and thus, valve assembly) can be crimped to a radially compressed state in an outer shaft and then expanded inside a patient by an inflatable balloon or equivalent expansion mechanism of the delivery apparatus. When the leaflet engagement frame comprises self-expandable materials, the leaflet engagement frame (and thus the valve assembly) can be crimped to a radially compressed state and restricted in the compressed state by a shaft, a capsule or equivalent mechanism of the delivery apparatus. Once inside the body, the valve assembly can be advanced from the outer shaft, which allows the valve assembly to expand to a partially expanded diameter, and further to the final functional diameter. Suitable plastically-deformable materials can include, but are not limited to, stainless steel, a nickel based alloy (e.g., a cobalt-chromium or a nickel-cobalt-chromium alloy such as MP35N alloy), polymers, or combinations thereof.

In some examples, the engagement frame first rung 176 includes only distally extending angled spikes 186, and is devoid of any other spikes extending in other orientations, such as in a proximal direction. In some examples, a single rung of angled struts of the leaflet engagement frame 170, namely the engagement frame first rung 176, includes the plurality of distally extending angled spikes 186, while any other rung thereof, if present, is devoid of distally extending angled spikes.

In some examples, leaflet engagement frame 170 further includes another rung of angled struts, distal to the engagement frame first rung 176, provided with a plurality of proximally extending angled spikes 188 extending solely from its struts. For example, the leaflet engagement frame 170^{a} illustrated in FIGS. 5 and 6B comprises the engagement frame fourth rung 182^{a}, which is also the distal-most rung at the outflow end of the leaflet engagement frame 170, defined by a plurality of fourth angled struts 183^{a} having proximally extending angled spikes 188^{a} that extend radially outward and in the proximal direction (i.e., upward or toward the outflow end). In some examples, each proximally extending angled spike 188 terminates with a sharp distal tip 189.

Stated otherwise, a sharp angle β (shown for example in FIG. 8B) is defined between the annular surface 175 and each proximally extending angled spike 188. The angle β can be similarly defined between each proximally extending angled spike 188 and the central longitudinal axis, meaning that the proximally extending angled spikes 188 are not parallel to the central longitudinal axis or any axis that extends in parallel to the central longitudinal axis. The angle β can be similar to, or different from, the angle α. In some examples, the angle β is in the range of 10-80 degrees. In some examples, the angle β is in the range of 20-70 degrees. In some examples, the angle β is in the range of 30-60 degrees.

Thus, while both the distally extending angled spikes 186 and the proximally extending angled spikes 188 are oriented radially outward, away from the central longitudinal axis, they extend from their respective angled struts in opposite directions, and in particular, toward each other.

When present, the proximally extending angled spikes 188 are configured to engage (and in some instances penetrate) the native annulus (such as the aortic annulus) or a base portion of the native of host leaflets (i.e., closer to the region of native leaflet attachment to the annulus or host leaflet attachment to the frame of a previously-implanted valve, in close proximity to the scalloped line of the previously-implanted valve). In contrast, the distally extending angled spikes 186 are configured to engage (and in some instances penetrate) the native or host leaflets at a region which is closer to their free ends (i.e., along their outflow portions).

When a leaflet engagement frame 170 includes more than two rungs of struts, such as one or more intermediate rungs disposed between the proximal and distal rungs of the outflow and inflow ends of the leaflet engagement frame 170, the intermediate rungs are preferably devoid of any type of angled spikes, such as either distally extending angled spikes 186 or proximally extending angled spikes 188. For example, while the engagement frame first rung 176^{a} includes a plurality of distally extending angled spikes 186, and the engagement frame fourth rung 182^{a} includes a plurality of proximally extending angled spikes 188, the two intermediate rungs of leaflet engagement frame 170^{a} disposed therebetween, namely the engagement frame second rung 178^{a} and the engagement frame third rung 180^{a} include relatively smooth second angled struts 179^{a} and third angled struts 181^{a}, respectively, which are devoid of either distally extending angled spikes 186 and/or proximally extending angled spikes 188. While two intermediate rungs are illustrated in the example of leaflet engagement frame 170^{a}, it is to be understood that any other number of intermediate rungs of struts is contemplated.

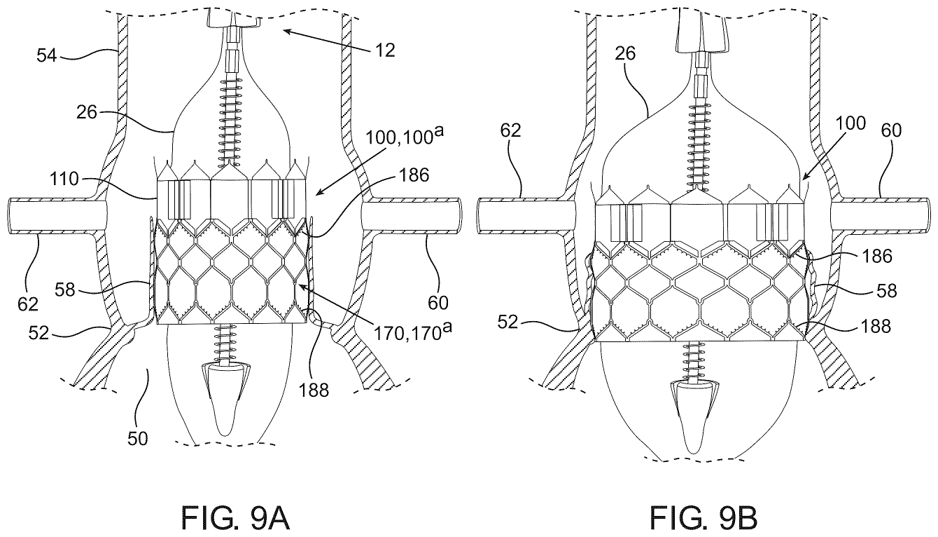

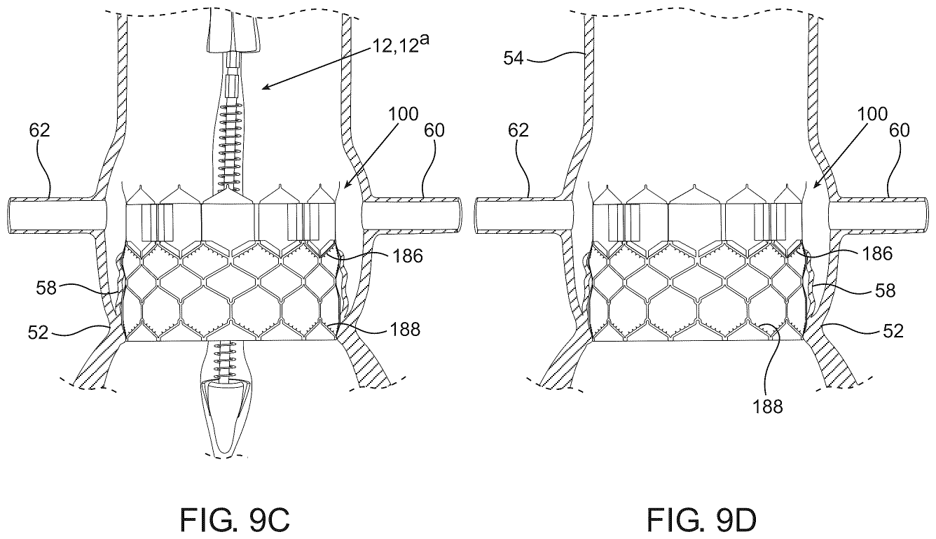

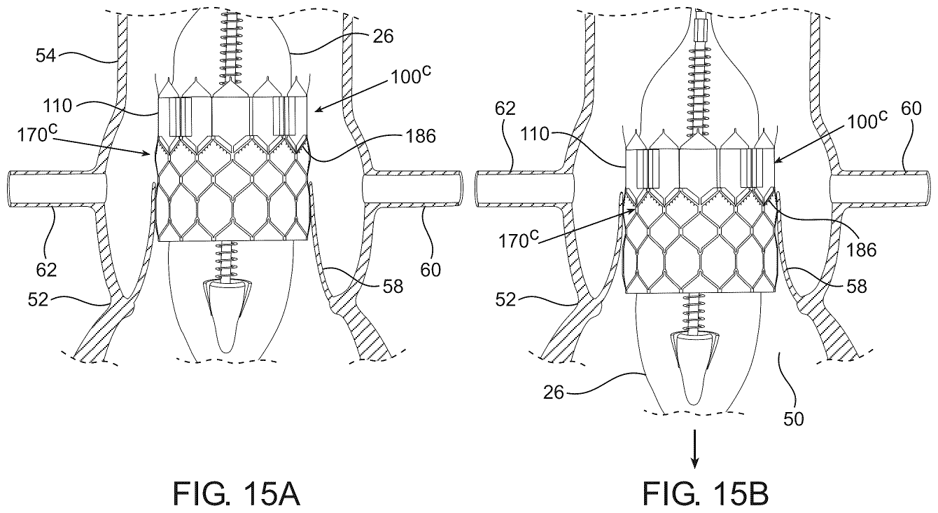

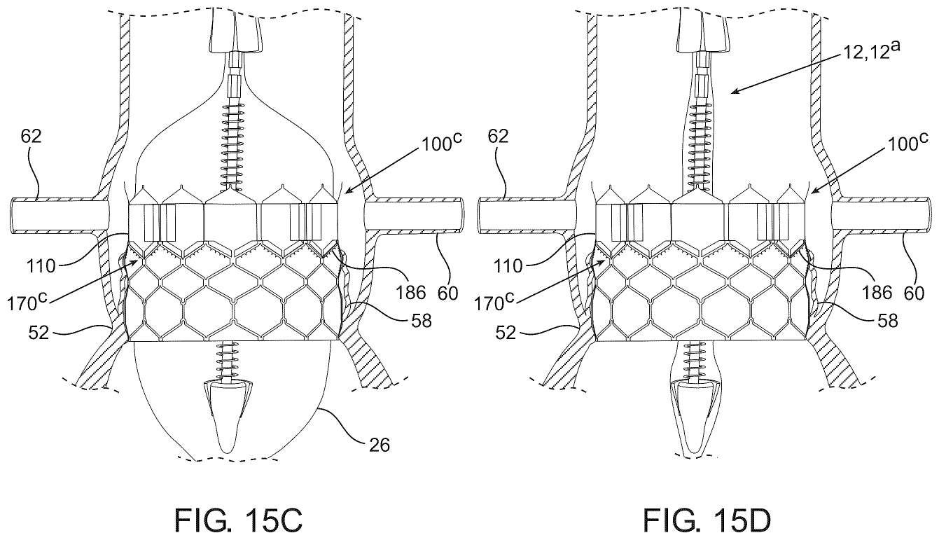

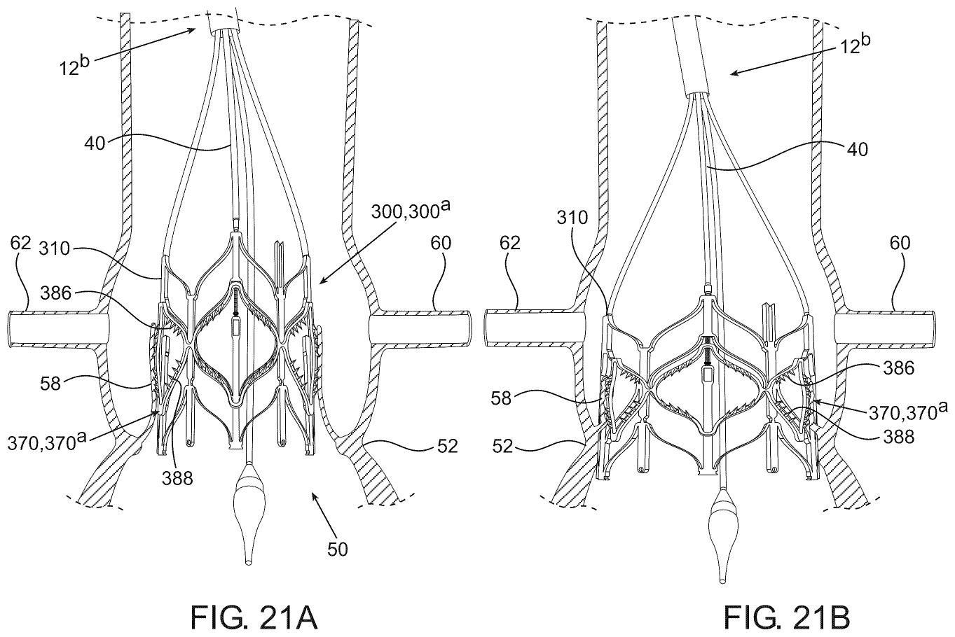

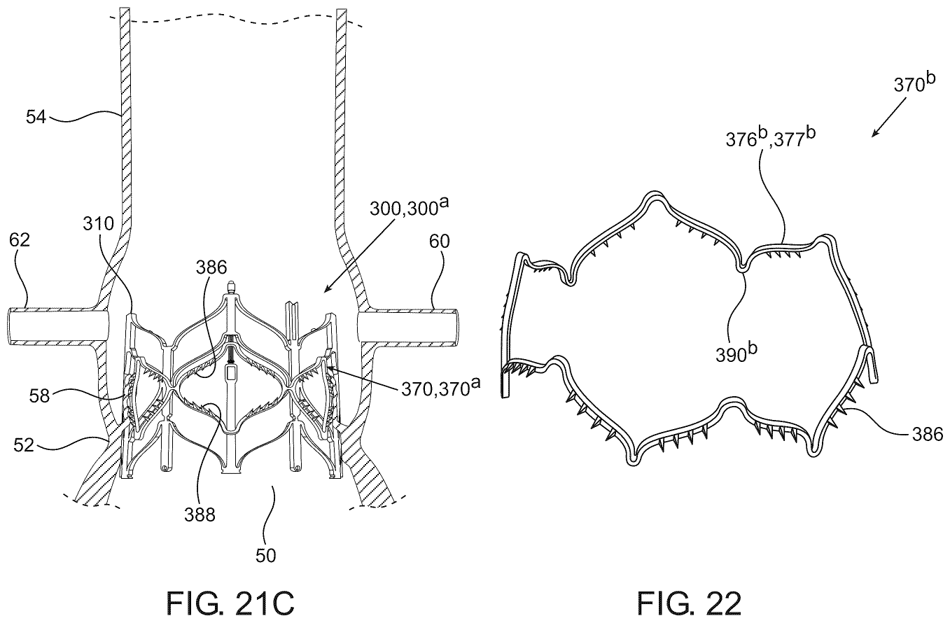

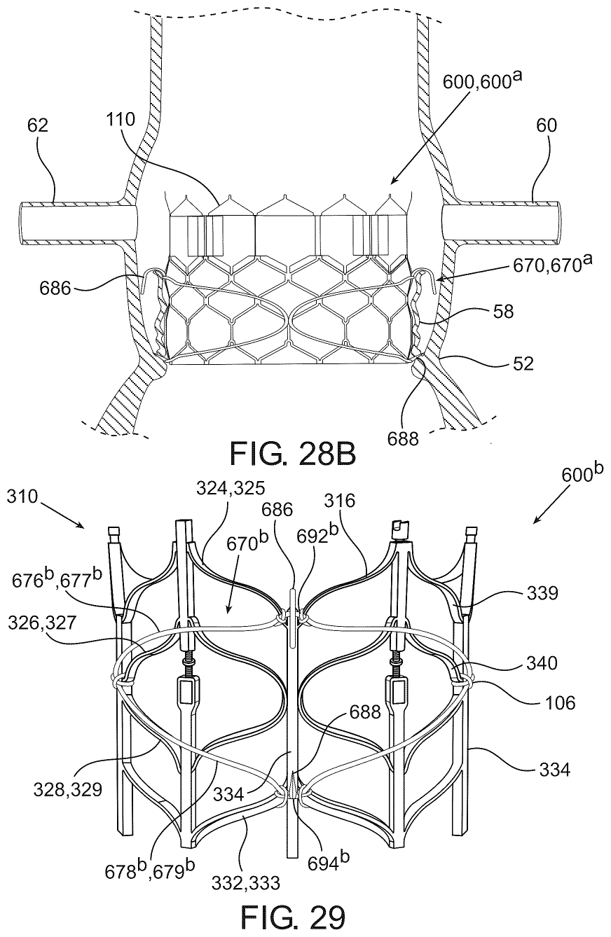

FIGS. 9A-D show stages of an exemplary method for folding native or host leaflets during implantation of a valve assembly 100, such as the valve assembly 100^{a}. For simplicity, inner or outer skirts are not shown. While valve assemblies may be described and shown throughout the current specification (such as in FIGS. 9A-D, FIGS. 15A-D, FIGS. 21A-C, FIGS. 28A-B) as being implanted in a native heart valve (e.g., the native aortic valve) so as to fold native leaflets 58, it is to be understood that the same methods are similarly applicable for ViV procedures, during which the valve assemblies can be implanted within a previously implanted prosthetic valve, so as to fold the host leaflets of the previously-implanted valve, mutatis mutandis.

The valve assembly 100 can be coupled to a delivery apparatus 12^{a}, which can be used to deliver, position, and secure the valve assembly 100 in a native heart valve annulus. In the illustrated implantation procedure, the valve assembly 100 is implanted in a native aortic annulus 52 using a transfemoral delivery approach. In other examples, the valve assembly 100 can be implanted at other locations (e.g., a mitral valve, a tricuspid valve, and/or a pulmonary valve), within previously-implanted prosthetic valve (i.e., during ViV procedures), and/or using other delivery approaches (e.g., transapical, transaortic, transseptal, etc.).

The valve assembly 100 can be releasably coupled to a distal end portion of a delivery apparatus 12^{a} by positioning the valve assembly 100 over an inflatable balloon 26 disposed at a distal end portion of the delivery apparatus, or over the balloon catheter 24 at a position proximal to the balloon 26 as described above, and radially compressing the valve assembly 100 to a crimped state. The distal end portion of the delivery apparatus 12^{a}, which comprises the balloon 26 and the radially compressed valve assembly 100, can be inserted percutaneously into a patient's vasculature and advanced toward the site of implantation. As shown in FIG. 9A, when the valve assembly 100 is disposed in or adjacent the aortic annulus 52, the balloon can be inflated to radially expand the prosthetic valve 110 (optionally after the crimped valve assembly 100 has been pushed by push shaft 22 over the balloon 26), along with the leaflet engagement frame 170 coupled thereto, from the crimped state.

As the valve assembly 100^{a} expands against the native leaflets 58, the proximally extending angled spikes 188 engage the leaflets 58 at their base portion, which can be a portion of the leaflets closer to the aortic annulus 52 in the case of native leaflets 58, or a portion of the leaflets closer to a scalloped line along which they are attached to a frame of a previously-implanted prosthetic valve for ViV procedures. In some examples, the proximally extending angled spikes 188 penetrate into the tissue of the native or host leaflets at their distal or base regions. In some examples, the proximally extending angled spikes 188 are engaged (i.e., pressed against, with or without penetration into) the heart annulus, such as the aortic annulus 52.

The distally extending angled spikes 186, in contrast, engage the leaflets 58 at the upper or proximal portions, closer to the free ends of the leaflets. In some examples, the distally extending angled spikes 186 penetrate into the tissue of the native or host leaflets at their proximal or upper regions.

FIG. 9A shows the valve assembly 100^{a} in a partially expanded state over a partially inflated balloon 26, wherein the proximally extending angled spikes 188 are engaged with the base portion of the native leaflets 58, while the distally extending angled spikes 186 are engaged with the upper portion of the leaflets 58, closer to their free ends. Engagement of either the distally extending angled spikes 186 and/or the proximally extending angled spikes 188 with the native or host leaflets, will start at a partially expanded state of the valve assembly 100, though each of the distally extending angled spikes 186 and/or the proximally extending angled spikes 188 can start engagement at a different partial expansion diameter.

In some examples, the proximally extending angled spikes 188 can engage the native or host leaflets or the surrounding annulus prior to engagement of the distally extending angled spikes 186 with the upper portions of the leaflets. In some examples, the proximally extending angled spikes 188 can engage the native or host leaflets or the surrounding annulus after engagement of the distally extending angled spikes 186 with the upper portions of the leaflets. In some examples, the proximally extending angled spikes 188 can engage the native or host leaflets or the surrounding annulus at the same time the distally extending angled spikes 186 engage the upper portions of the leaflets.

Further expansion of the valve assembly 100 to the functional size of the prosthetic valve 110 serves to fold the native or host leaflets. FIG. 9B shows the fully expanded valve assembly 100^{a} over the fully inflated balloon 26. During this expansion to the prosthetic valve's functional diameter, both the valve frame 116 and the leaflet engagement frame 170 foreshorten in the axial direction, such that the outflow end of the leaflet engagement frame 170, defined by the engagement frame first rung 176 with the distally extending angled spikes 186, moves distally toward the inflow end of the leaflet engagement frame 170, which is defined by the engagement frame fourth rung 182^{a} in the case of leaflet engagement frame 170^{a}.

The proximally extending angled spikes 188 are held in an axial position relative to the aortic annulus 52 (or relative to the scalloped line along which host leaflets are attached to the frame of a previously implanted valve, for ViV procedures) during radial expansion of the valve assembly 100, which allows the distally extending angled spikes 186 to move distally and drag the proximal portions of the native or host leaflets therewith, thereby folding them distally/downward and away from the ostia of the coronary arteries 60, 62.

Once a selected diameter of the valve assembly 100 is reached, the balloon 26 of the delivery apparatus 12^{a} can be deflated, as shown in FIG. 9C. The delivery apparatus 12^{a} can then be withdrawn from the patient's body, leaving the valve assembly 100 within the aortic annulus 52 to regulate blood flow from the left ventricle 50 into the aorta 54 as shown in FIG. 9D. As further shown in FIG. 9D, the native or host leaflet around the implanted valve assembly 100 are retained distal of the ostia of the coronary arteries 60, 62 with the distally extending angled spikes 186, thus reducing the possibility of the native leaflets 58 fully or partially covering the coronary ostia. The native leaflets 58 may have been displaced distal of the proximal ends of the valve leaflets 152. Upon implantation, the valve leaflets 152 operate as prosthetic replacements for the native leaflets 58.

FIG. 1A shows a conventional outer skirt 144 of a prosthetic valve 110, extending all the way to the valve inflow end 114. Since a major role of the outer skirt 144 is to provide PVL sealing against the native anatomy, when a leaflet engagement frame 170 is coupled to the valve frame 116 to form a valve assembly 100, the outer skirt 144 can be mounted, in some examples, on the outer surface of the valve assembly 100, which can include portions of the outer surface of leaflet engagement frame 170 as well as portions of the outer surface of the valve frame 116. However, in some configurations, and specifically when a valve assembly 100^{a} includes a leaflet engagement frame 170^{a} that comprises an engagement frame fourth rung 182^{a} aligned with valve frame inflow rung 132, an outer skirt extending all the way to the inflow end of the valve may conceal the proximally extending angled spikes 188 and prevent them from properly engaging the native or host leaflets.

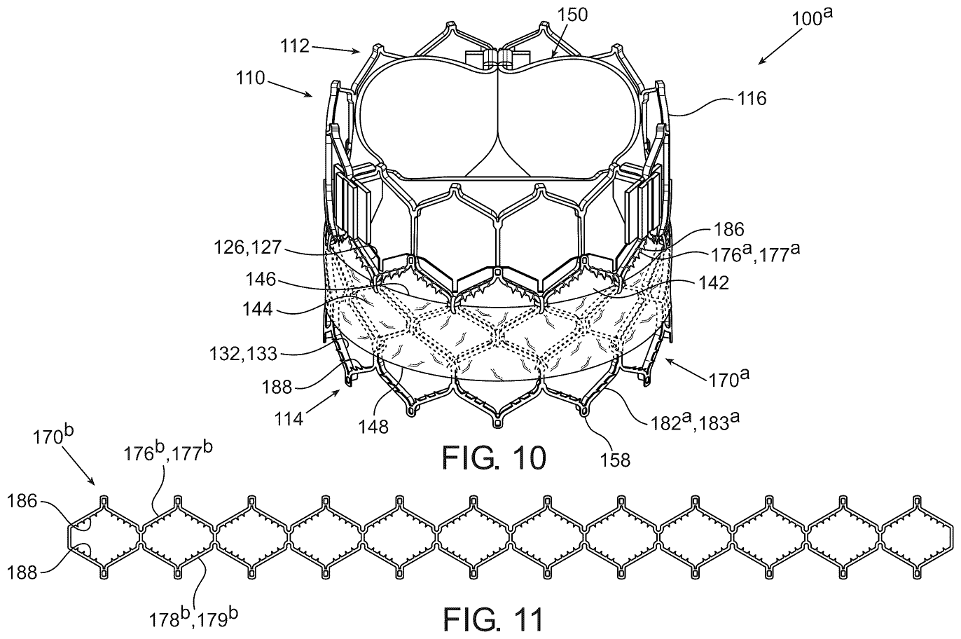

In some examples, as shown in FIG. 10, the outer skirt 144, which is mounted over the outer surface of the leaflet engagement frame 170^{a} (without contacting any portion of the outer surface of the valve frame 116 in the illustrated example), extends between an outer skirt outflow end 146 and an outer skirt inflow end 148 such that the outer skirt outflow end 146 is distal to the distally extending angled spikes 186, and the outer skirt inflow end 148 is proximal to the proximally extending angled spikes 188. In this manner, an outer skirt 144 can be mounted over the leaflet engagement frame 170 and function as a sealing member against the native anatomy, without concealing any of the spikes of the leaflet engagement frame 170.

FIG. 11 shows another example of a leaflet engagement frame 170^{b} which is similar to any example of leaflet engagement frame 170^{a}, except that it includes only two rungs of struts, namely an engagement frame first rung 176^{b} comprising distally extending angled spikes 186, and an engagement frame second rung 178^{b} comprising proximally extending angled spikes 188, and is devoid of any intermediate rungs therebetween, such that the engagement frame first and second rungs together define a single row of engagement frame cells 172^{b}.

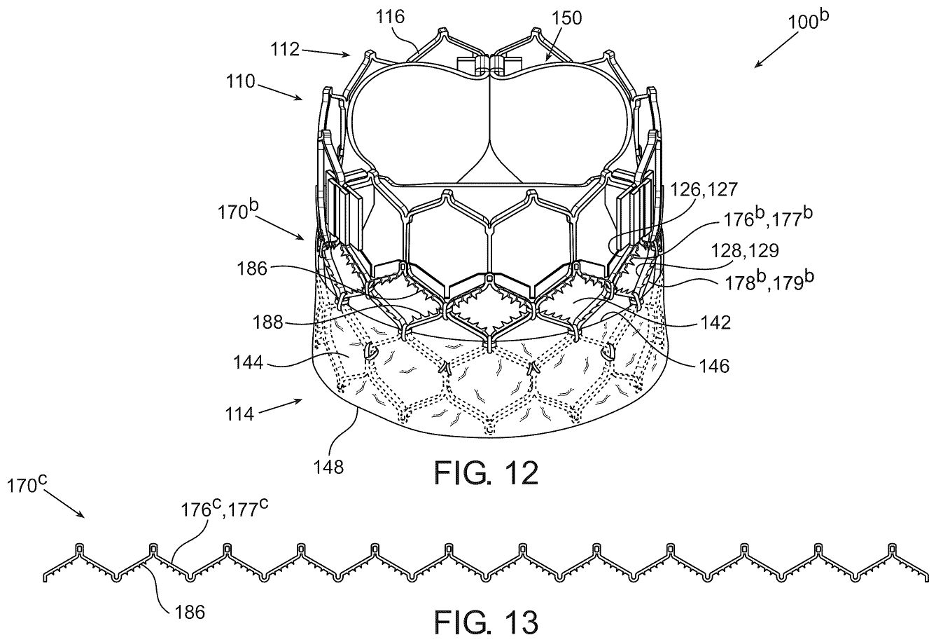

FIG. 12 shows an example of a valve assembly 100^{b} comprising the leaflet engagement frame 170^{b} coupled to the valve frame 116, such that the first angled struts 177^{b} of the engagement frame first rung 176^{b} are aligned with the angled struts 127 of the valve frame first intermediate rung 126, and the second angled struts 179^{b} of the engagement frame second rung 178^{b} are aligned with the angled struts 129 of the valve frame second intermediate rung 128.

In some examples, as shown in FIG. 12, the outer skirt outflow end 146 is distal to the proximally extending angled spikes 188 (and therefore, distal to the distally extending angled spikes 186 as well), while the outer skirt inflow end 148 can extend all the way toward the valve inflow end 114.

FIG. 13 shows another example of a leaflet engagement frame 170^{c} which is similar to any example of leaflet engagement frames 170^{a} or 170^{b}, except that it includes only a single rung struts, namely an engagement frame first rung 176^{c} comprising distally extending angled spikes 186, and is devoid of proximally extending angled spikes 188.

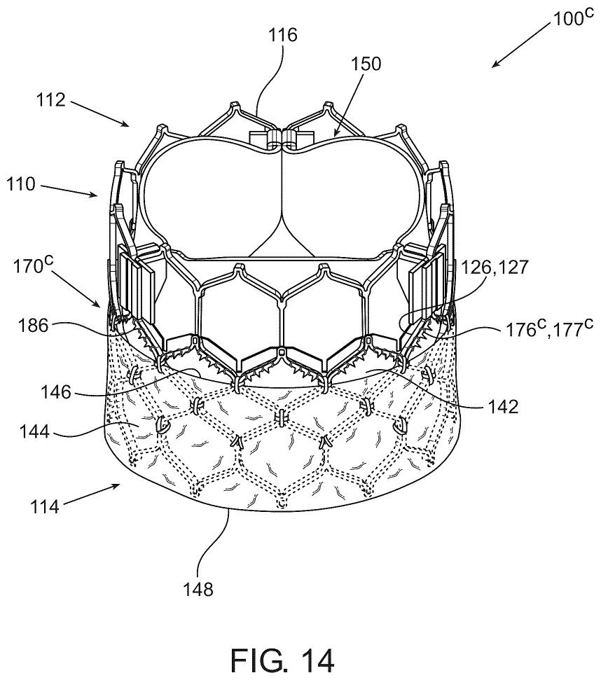

FIG. 14 shows an example of a valve assembly 100^{c} comprising the leaflet engagement frame 170^{c} coupled to the valve frame 116, such that the first angled struts 177^{c} of the engagement frame first rung 176^{c} are aligned with the angled struts 127 of the valve frame first intermediate rung 126. In some examples, as further shown in FIG. 14, the outer skirt outflow end 146 is distal to the distally extending angled spikes 186, while the outer skirt inflow end 148 can extend all the way toward the valve inflow end 114.

Since a valve assembly 100^{c} comprising the leaflet engagement frame 170^{c} is devoid of proximally extending angled spikes 188, mere expansion of the valve assembly 100^{c} against the native or host leaflets may not result in the desired folded configuration of the leaflets, due to the lack of engagement features that provide a counter force retaining the valve assembly in position while the upper portions of the leaflets should be folded distally/downward. In such cases, the delivery apparatus may be utilized to assist in proper positioning and folding of the leaflets.

FIGS. 15A-D show stages of an exemplary method for folding native or host leaflets during implantation of a valve assembly 100^{c} that comprises distally extending angled spikes 186, and can be devoid of proximally extending angled spikes 188. For simplicity, inner or outer skirts are not shown. The valve assembly 100^{c} can be carried over the delivery apparatus 12^{a} toward the site of implantation in the same manner described above in conjunction with FIGS. 9A-D. FIG. 15A shows the valve assembly 100^{c} in a partially expanded state, over a partially expanded balloon, such that the distally extending angled spikes 186 are engaged with the native or host leaflets as described above.

Once the distally extending angled spikes 186 are engaged with the leaflets, the delivery apparatus is utilized to push the valve assembly 100^{c} distally (for example, toward the left ventricle 50), in this partially expanded state of the valve assembly 100^{c}, as shown in FIG. 15B. This distal displacement of the entire valve assembly 100^{c} carries the inflow ends of the native leaflets therewith in the same direction, away from the ostia of the coronary arteries 60, 62. Once the native or host leaflets are positioned as desired, the valve assembly 100^{c} can be further expanded to its final functional diameter (for example, by further inflating the balloon 26), as shown in FIG. 15C, after which the balloon 26 can be deflated and the delivery apparatus 12^{a} can be retrieved from the patient's body, leaving the valve assembly 100^{c} implanted in position with the native leaflets folded away from the ostia of the coronary arteries, as shown in FIG. 15D.

In some examples, there is provided a prosthetic valve 210 comprising distally extending angled spikes 286 integrally formed with a single rung of struts of the valve frame 216, and optionally proximally extending angled spikes 288, which are integrally formed with another single rung of struts of the valve frame 216, such that the prosthetic valve can be utilized in a similar manner to that described above for valve assemblies 100, but without being coupled to a leaflet engagement frame. The prosthetic valve 210 is similar in all other respects to prosthetic valve 110, with similar reference numerals referring to similar features, and in the interest of brevity will not be described further.

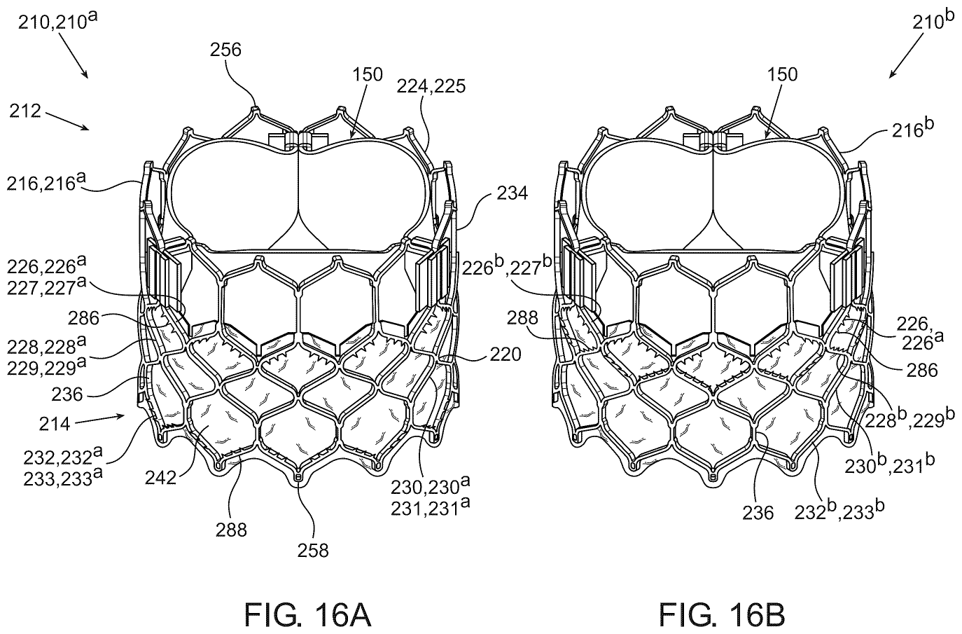

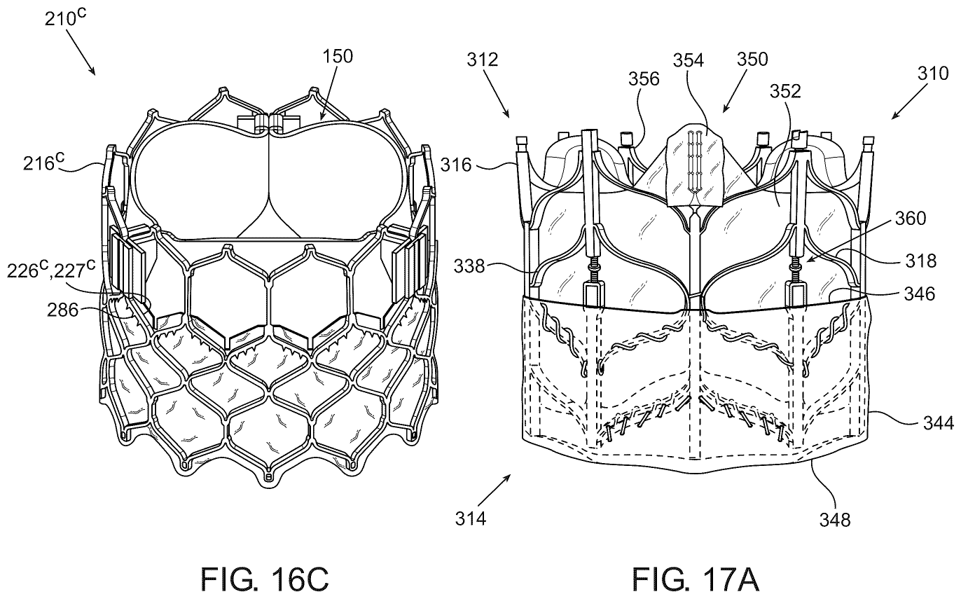

FIG. 16A shows an example of a prosthetic valve 210^{a}, which can be similar to prosthetic valve 110, except that it comprises a plurality of distally extending angled spikes 286 extending from angled struts 227^{a} of a valve frame first intermediate rung 226^{a}, and a plurality of proximally extending angled spikes 288 extending from angled struts 233^{a} of a valve frame inflow rung 232^{a}.

The distally extending angled spikes 286 may be implemented to extend from the valve frame first intermediate rung 226^{a} in the same manner described for any example of the distally extending angled spikes 186 extending from the first rung 176^{a} of leaflet engagement frame 170^{a}. The proximally extending angled spikes 288 may be implemented to extend from the valve frame inflow rung 232^{a} in the same manner described for any example of the proximally extending angled spikes 188 extending from the fourth rung 182^{a} of leaflet engagement frame 170^{a}. The rest of the intermediate rungs, such as the valve frame second intermediate rung 228^{a} and the valve frame third intermediate rung 230^{a}, remain devoid of any type of angled spikes, in the same manner described above for engagement frame second rung 178^{a} and engagement third rung 180^{a}.

An outer skirt 244 can extend around the outer surface of the valve frame 216^{a} according to any example described above for an outer skirt 144 of valve assembly 100^{a}, including an example in which the outer skirt outflow end 246 is distal to the distally extending angled spikes 286, and the outer skirt inflow end 248 is proximal to the proximally extending angled spikes 288 (outer skirt 244, outer skirt outflow end 246 and outer skirt inflow end 248 are not shown in FIG. 16A, but can be generally similar to outer skirt 144, outer skirt outflow end 146 and outer skirt inflow end 148, respectively, shown in FIG. 1A).

The prosthetic valve 210^{a} can be delivered by a delivery apparatus 12^{a} and implanted following the same steps described hereinabove in conjunctions with FIGS. 9A-D, mutatis mutandis, so as to fold the native or host leaflets to prevent obstruction of the ostia of the coronary arteries.

FIG. 16B shows another example of a prosthetic valve 210^{b}, which can be similar to prosthetic valve 110^{b}, except that it comprises a plurality of distally extending angled spikes 286 extending from angled struts 227^{b} of a valve frame first intermediate rung 226^{b}, and a plurality of proximally extending angled spikes 288 extending from angled struts 229^{a} of a valve frame second intermediate rung 228^{b}.

The distally extending angled spikes 286 may be implemented to extend from the valve frame first intermediate rung 226^{b} in the same manner described for any example of the distally extending angled spikes 186 extending from the first rung 176^{b} of leaflet engagement frame 170^{b}. The proximally extending angled spikes 288 may be implemented to extend from the valve frame second intermediate rung 228^{b} in the same manner described for any example of the proximally extending angled spikes 188 extending from the second rung 178^{b} of leaflet engagement frame 170^{b}. The rest of the strut rungs of prosthetic valve 210^{b} remain devoid of any type of angled spikes.

The outer skirt 244 can extend around the outer surface of the valve frame 216^{b} in a similar manner to that described for valve assembly 100^{b}, for example such that the outer skirt outflow end 246 is distal to both the distally extending angled spikes 286 and the proximally extending angled spikes 288. The prosthetic valve 210^{b} can be utilized during implantation for folding native or host leaflets in the same manner as that of valve assembly 100^{b}, mutatis mutandis.

FIG. 16C shows another example of a prosthetic valve 210^{c}, which can be similar to prosthetic valve 110^{c}, except that it comprises a plurality of distally extending angled spikes 286 extending from angled struts 227^{b} of a valve frame first intermediate rung 226^{b}, and unlike the examples of prosthetic valves 210^{c} and 210^{b}, prosthetic valve 210^{c} is devoid of proximally extending angled spikes.

The distally extending angled spikes 286 may be implemented to extend from the valve frame first intermediate rung 226^{c} in the same manner described for any example of the distally extending angled spikes 186 extending from the first rung 176^{c} of leaflet engagement frame 170^{c}. All other strut rungs of prosthetic valve 210^{c} remain devoid of any type of angled spikes.

The outer skirt 244 (not shown in FIG. 16C for clarity) can extend around the outer surface of the valve frame 216^{c} in a similar manner to that described for valve assembly 100^{c}, for example such that the outer skirt outflow end 246 is distal to the distally extending angled spikes 286.

The prosthetic valve 210^{c} can be delivered by a delivery apparatus 12^{a} and implanted following the same steps described hereinabove in conjunctions with FIGS. 15A-D, mutatis mutandis, so as to fold the native or host leaflets to prevent obstruction of the ostia of the coronary arteries.

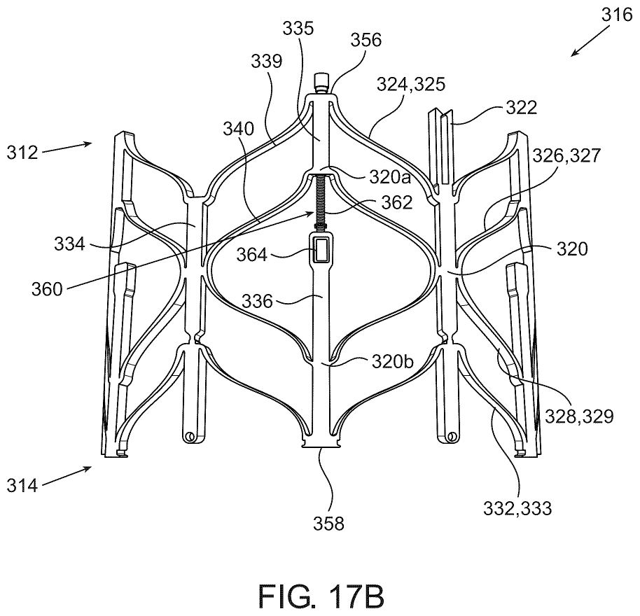

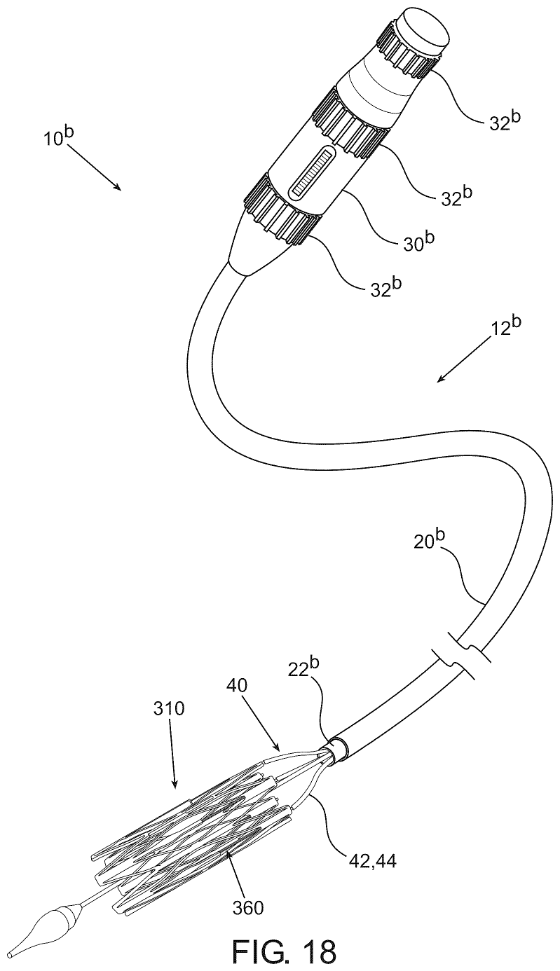

FIGS. 17A and 17B show perspective views of a mechanically expandable prosthetic valve 310 with and without soft components attached thereto, according to some examples. FIG. 18 shows a perspective view of a delivery assembly 10^{b} that can include a delivery apparatus 12^{b} for use with a mechanically expandable prosthetic device, such as mechanically expandable prosthetic valve 310, as well as prosthetic valve 510 or mechanically expandable valve assemblies 300, 400, 600 which will be described in further detail below, according to some examples.

Similar to prosthetic valve 110, the prosthetic valve 310 includes an annular valve frame 316 which can be a unitary lattice frame made of intersecting frame struts 318, defined between a valve outflow end 312 and a valve inflow end 314, and a leaflet assembly 350 mounted within the valve frame 316. The leaflets assembly 350, as well as an inner skirt 342 (hidden from view in FIG. 17A, but can be functionally similar to inner skirt 142 of FIG. 1A)) or an outer skirt 344, can be similar in all respect to leaflet assembly 150, inner skirt 142 and outer skirt 144, and in the interest of brevity will not be further described.

Unlike prosthetic valve 110, which can be representative of a balloon expandable valve, or alternatively a self-expandable valve, the prosthetic valve 310 can be expanded utilizing a mechanical mechanism. For example, the prosthetic valve 310 can be radially expanded by maintaining the valve inflow end 314 at a fixed position while applying a force in the axial direction against the valve outflow end 312 toward the inflow end 314. Alternatively, the prosthetic valve 310 can be expanded by applying an axial force against the valve inflow end 314 while maintaining the valve outflow end 312 at a fixed position, or by applying opposing axial forces to the inflow and outflow ends 314, 312, respectively.

Similar to a delivery apparatus 12^{a} described above for use with a balloon-expandable prosthetic valve 110, a delivery apparatus 12^{b} can include a handle 30^{b}, and an outer shaft 20^{b}, as well as an optional additional delivery shaft 28 that can be disposed within the outer shaft 20^{b} and can be optionally axially movable relative thereto. However, instead of a balloon catheter 24 and inflatable balloon 26, the delivery apparatus 12^{b} includes a plurality of actuation assemblies 40, configured to radially expand and/or radially compress the prosthetic valve 310 (or prosthetic valve 510) when actuated.

As shown in FIGS. 17A-B, the prosthetic valve 310 can include one or more actuators 360 mounted to and equally spaced around the inner surface of the valve frame 316. Each of the actuators 360 can be configured to form a releasable connection with a respective actuation assembly 40.

The valve frame 316 of the prosthetic valve 310 illustrated in FIG. 17B comprises rungs of curved struts 325, 327, 329, 333, and axial struts or posts 334, 335, 336. The curved struts define a plurality of cells 338 extending circumferentially around the valve frame 316. While only one side of the valve frame 316 is illustrated in FIG. 17B, it should be appreciated that the valve frame 316 forms an annular structure having an opposite side that is substantially identical to the portion shown. In the illustrated example, the valve frame 316 comprises a valve frame outflow rung 324 of curved struts 325 defining the valve outflow end 312; a valve frame first intermediate rung 326 of curved struts 327; a valve frame second intermediate rung 328 of curved struts 329; and a valve frame inflow rung 332 of curved struts 333 defining the valve inflow end 314.

The cells 338 can include first cells 339 and second cells 340. Each first cell 339 can have an axially-extending elliptical shape including outflow apex 356 and inflow apex 358 disposed at the major vertices of the ellipse. Each first cell 339 can further comprise a respective second cell 340 disposed within the outer perimeter of the first cell 339. The second cell 340 can have a circumferentially-extending elliptical shape including a proximal junction 320^{a} and a distal junction 320^{b} disposed at the minor vertices of the ellipse. While illustrated as elliptical, it is to be understood that any of the cells 338 can have any of various other shapes, for example, hexagonal, triangular, tear drop shaped, rectangular, square, square-oval, etc.

As mentioned, the frame can comprise a plurality of axially-extending struts or posts, including a plurality of proximal posts 335 and distal posts 336. The proximal posts 335 (shown as the upper posts in the illustrated example) can extend to the valve outflow end 312, and the distal posts 336 (shown as the lower posts in the illustrated example) can extend to the valve inflow end 314. Each proximal post 335 can be axially aligned with a corresponding distal post 336 for a pair of proximal and distal posts. One or more pairs of proximal and distal posts 335, 336 can be configured as actuators 360. The valve frame 316 can further comprise additional axial support posts 334 disposed between each pair of adjacent circumferentially disposed first cells 339, and the actuators 360 can be disposed such that they extend through and are coupled to the apices 356, 358 and valve frame junctions 320^{a}, 320^{b} through the first and second cells. The axial support posts 334 can be coupled together via curved struts 325, 327, 329, 333.

Each first cell 339 is formed by two curved struts 325 of the valve frame outflow rung 324 and two curved struts 333 of the valve frame inflow rung 332. Each curved strut 325 is coupled on one end to a proximal post 335 of an actuator 360 and on the other end to an axial support post 334. Each curved strut 333 is coupled on one end to a distal post 336 of an actuator 360 and on the other end to an axial support post 334.

Each second cell 340 is formed by two curved struts 327 of the valve frame first intermediate rung 326 and two curved struts 329 of the valve frame second intermediate rung 328. The lower/distal ends of the curved struts 327 and the upper/proximal ends of the curved struts 329 can be connected to the axial support posts 334. The upper/proximal ends of the curved struts 327 can be connected to a proximal post 335 of a respective actuator 360. The lower/distal ends of the curved struts 329 can be connected to a distal post 336 of the respective actuator 360.

Each proximal post 335 can extend through and be coupled to outflow apex 356 and proximal junction 320^{a} of a respective first and second cell pair. Each distal post 336 can extend through and be coupled to inflow apex 358 and distal junction 320^{b} of the respective first and second cell pair. In the illustrated example, the valve frame 316 comprises six first cells 339 extending circumferentially in a row, with a second cell 340 within each first cell 339, and six pairs of proximal and distal posts 335, 336 coupled to a respective pair of cells 339, 340. However, in other examples, the valve frame 316 can comprise a greater or fewer number of first cells 339 within a row, and a correspondingly greater or fewer number of second cells 340 and/or pairs of posts 335, 336.

In some examples, each pair of posts 335, 336 can be configured as an actuator 360. For example, in the illustrated implementation, each of the six pairs of posts 335, 336 is configured as an actuator 360. In other examples, not all pairs of posts 335, 336 need be actuators. Where a pair of posts 335, 336 is configured as an actuator, a threaded rod 362 extends through each post of 339, 340 of the pair to effect radial compression and expansion of the frame. The distal post 336 can comprise a threaded nut 364 disposed at a proximal end portion thereof and configured to engage the threaded rod 362. Rotation of the threaded rod 362 in a first direction (e.g., clockwise) can cause corresponding axial movement of the proximal and distal posts 335, 336 toward one another, expanding the valve frame 316, and rotation of the threaded rod 362 in a second direction (e.g., counterclockwise) causes corresponding axial movement of the proximal and distal posts 335, 336 away from one another, compressing the frame. As the valve frame 316 moves from a compressed state to an expanded state, the gap between the proximal and distal posts 335, 336 can narrow.

Because the threaded rod 362 is secured to the valve frame 316 at axially spaced locations (the valve outflow end 312 and the valve inflow end 314) rotating the threaded rod 362 causes axial movement of the outflow end 312 and inflow end 314 relative to one another to cause radial expansion or compression of the valve frame 316. For example, moving the outflow and inflow ends 312, 314 toward one another causes the valve frame 316 to foreshorten axially and expand radially.

As shown in FIG. 17B, the axial support posts 334 can extend longitudinally and can include commissure support member, such as commissure windows 322, at proximal portions thereof. Each axial support post 334 can also extend toward the valve inflow end 314 and serve to prevent or mitigate portions of the outer skirt 344 from extending radially inwardly. The distal portions of the axial support posts 334 can further serve as supports to which portions of the inner and/or outer skirts 342, 344 can be coupled.

As mentioned, FIG. 17B shows only one side of the valve frame 316. Though only one axial support post 334 comprising a commissure window 322 is shown in FIG. 17B, it should be noted that the valve frame 316 can comprise any number of axial support posts 334, any number of which can include commissure windows 322. For example, a valve frame 316 can comprise six axial support posts 334, three of which also include commissure windows 322. In some implementations, for example, a valve frame can comprise one, two, three, or four commissure windows.

The delivery apparatus 12^{b} includes a plurality of actuation assemblies 40 that can be releasably coupled to the prosthetic valve 310. For example, each actuation assembly 40 can be coupled to a respective actuator 360 of the prosthetic valve 310. Each actuation assembly 40 can comprise a support tube or sleeve 44 and a driver 42 extending through the lumen of the sleeve 44. The actuation assemblies can be at least partially disposed radially within, and extend axially through, one or more lumens of the outer shaft 20^{b} or delivery shaft 28.

Each threaded rod 362 can include a head portion (not shown) configured to be releasably coupled to a respective actuation assembly 40. The head portion of the threaded rod 362 abuts the valve outflow end 312 and can be used to apply a distally-directed force to the proximal post, for example, during radial expansion of the valve frame 316. The distal end portions of the sleeve 44 and driver 42 can be configured to engage or abut the proximal end (e.g., the outflow end) of the threaded rod 362 and/or the valve frame 316. The proximal portions of the sleeve 44 and driver 42 can be operatively coupled to the handle 30^{b} of delivery apparatus 12^{b}. The handle 30^{b} can be operated to cause rotation of the drivers 42, which translated to a corresponding rotation of the threaded rods 362. Rotation of the threaded rods 362 causes axial movement of the valve inflow end 314 and the valve outflow end 312 relative to one another to cause radial expansion (or compression) of the valve frame 316.

When the prosthetic valve 310 is implanted at a selected implantation site within a patient, the patient's native anatomy (e.g., the native aortic annulus) may exert radial forces against the prosthetic valve 310 that would tend to compress the valve frame 316. However, the engagement of the threaded rod 362 with the threaded nut 364 prevents such forces from compressing the valve frame 316, thereby ensuring that the frame remains locked in the desired radially expanded state.

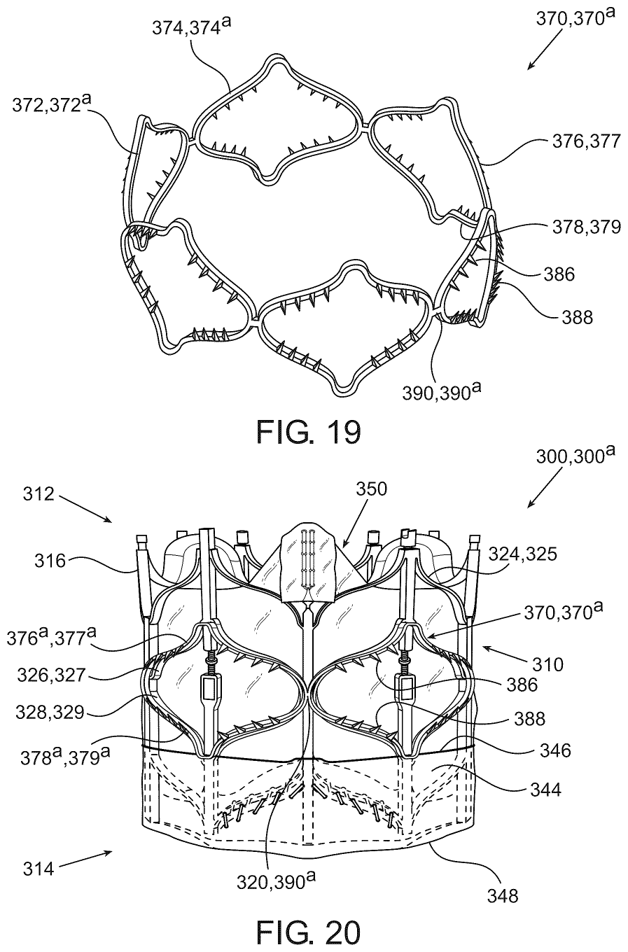

FIG. 19 shows an example of a leaflet engagement frame 370 that can be coupled to a prosthetic valve 310 to form a valve assembly 300, shown for example in FIG. 20. The leaflet engagement frame 370 can be similar to the various examples described above for leaflet engagement frames 170, and comprise at least one rung of struts provided with a plurality of distally extending angled spikes 386. In some examples, the leaflet engagement frame 370 further comprises another rung of struts provided with proximally extending angled spikes 388, opposite to the distally extending angled spikes 386.

The leaflet engagement frame 370 comprises a plurality of engagement frame struts 374 configured in an annular shape. The engagement frame struts 374 define at least one rung, which can be referred to as an engagement frame first rung 376 defined by a plurality of first curved struts 377 at the proximal or outflow end of the leaflet engagement frame 370. The first curved struts 377 comprise distally extending angled spikes 386 that extend radially outward and in the distal direction (i.e., downward or toward the inflow end), and can be identical to any example described above for distally extending angled spikes 186.

An example of leaflet engagement frame 370^{a}, shown in FIG. 19, comprises an engagement frame first rung 376^{a} of circumferentially extending, first curved struts 377^{a}, and an engagement frame second rung 378^{a} of circumferentially extending, second curved struts 379^{a}. The engagement frame struts 374^{a} collectively define a single row of engagement frame cells 372^{a}.

In some examples, the engagement frame first rung 376 includes only distally extending angled spikes 386, and is devoid of any other spikes extending in other orientations, such as in a proximal direction. In some examples, a single rung of curved struts of the leaflet engagement frame 370, namely the engagement frame first rung 376, includes the plurality of distally extending angled spikes 386. In some examples, leaflet engagement frame 370 further includes another rung of curved struts, distal to the engagement frame first rung 376, provided with a plurality of proximally extending angled spikes 388 that can be identical to any example described above for proximally extending angled spikes 188. For example, the leaflet engagement frame 370^{a} illustrated in FIG. 19 comprises distally extending angled spikes 386 extending from the first curved struts 377^{a} of the first rungs 376^{a}, and proximally extending angled spikes 388 extending from the second curved struts 379^{a} of the second rung 378^{a}.

FIG. 20 shows the leaflet engagement frame 370^{a} mounted over the prosthetic valve 310, together forming the valve assembly 300^{a}. The leaflet engagement frame 370^{a} is configured such that the curved struts of the engagement frame first rung 376^{a} and second rung 378^{a}, align with the curved struts of the valve frame first intermediate rung 326 and second intermediate rung 328, respectively. This in turn results in the single row of engagement frame cells 372^{a} being aligned with the row of second cells 340 of the prosthetic valve 310. The leaflet engagement frame 370 can be coupled to the prosthetic valve 310 in the same manner described in any one of the examples for coupling leaflet engagement frame 170 to prosthetic valve 110.

In some examples, as further shown in FIG. 20, the outer skirt 344 is disposed around the outer surface of the leaflet engagement frame 370^{a} such that the outer skirt outflow end 346 is distal to both the distally extending angled spikes 386 and the proximally extending angled spikes 388, while the outer skirt inflow end 348 can extend all the way to the valve inflow end 314.

FIGS. 21A-C show stages of an exemplary method for folding native or host leaflets during implantation of a valve assembly 300, such as the valve assembly 300^{a}. For simplicity, inner or outer skirts are not shown. The valve assembly 300 can be coupled to a delivery apparatus 12^{b}, which can be used to deliver, position, and secure the valve assembly 300 in a native heart valve annulus. In the illustrated implantation procedure, the valve assembly 300 is implanted in a native aortic annulus 52 using a transfemoral delivery approach. In other examples, the valve assembly 300 can be implanted at other locations (e.g., a mitral valve, a tricuspid valve, and/or a pulmonary valve), within previously-implanted prosthetic valve (i.e., during ViV procedures), and/or using other delivery approaches (e.g., transapical, transaortic, transseptal, etc.).

The valve assembly 300 can be releasably coupled, as described above, to the actuation assemblies 40 of delivery apparatus 12^{b}, and advanced in a compressed state through the patient's vasculature toward the site of implantation (e.g., the aortic annulus). As shown in FIG. 21A, when the valve assembly 300 is disposed in or adjacent the aortic annulus 52, the actuation assemblies 40 can be utilized, as described above, to radially expand the prosthetic valve 310, along with the leaflet engagement frame 370 coupled thereto, from the crimped state.

As the valve assembly 300^{a} expands against the native leaflets 58, the proximally extending angled spikes 388 engage the leaflets 58 at their base portion, which can be a portion of the leaflets closer to the aortic annulus 52 in the case of native leaflets 58, or a portion of the leaflets closer to a scalloped line along which they are attached to a frame of a previously-implanted prosthetic valve for ViV procedures. In some examples, the proximally extending angled spikes 388 penetrate into the tissue of the native or host leaflets at their distal or base regions. In some examples, the proximally extending angled spikes 388 are engaged (i.e., pressed against, with or without penetration into) the heart annulus, such as the aortic annulus 52.

The distally extending angled spikes 386, in contrast, engage the leaflets 58 at the upper or proximal portions, closer to the free ends of the leaflets. In some examples, the distally extending angled spikes 386 penetrate into the tissue of the native or host leaflets at their proximal or upper regions.

FIG. 21A shows the valve assembly 300^{a} in a partially expanded state, wherein the proximally extending angled spikes 388 are engaged with the base portion of the native leaflets 58, while the distally extending angled spikes 386 are engaged with the upper portion of the leaflets 58, closer to their free ends. Engagement of either the distally extending angled spikes 386 and/or the proximally extending angled spikes 388 with the native or host leaflets, will start at a partially expanded state of the valve assembly 300, though each of the distally extending angled spikes 386 and/or the proximally extending angled spikes 388 can start engagement at a different partial expansion diameter.

In some examples, the proximally extending angled spikes 388 can engage the native or host leaflets or the surrounding annulus prior to engagement of the distally extending angled spikes 386 with the upper portions of the leaflets. In some examples, the proximally extending angled spikes 388 can engage the native or host leaflets or the surrounding annulus after engagement of the distally extending angled spikes 386 with the upper portions of the leaflets. In some examples, the proximally extending angled spikes 388 can engage the native or host leaflets or the surrounding annulus at the same time the distally extending angled spikes 386 engage the upper portions of the leaflets.

Further expansion of the valve assembly 300 to the functional size of the prosthetic valve 310 serves to fold the native or host leaflets. FIG. 21B shows the fully expanded valve assembly 300^{a} by applying further rotational movement to the threaded rods 362 of the actuators 360 via the drivers 42 of the actuation assemblies 40. During this expansion to the prosthetic valve's functional diameter, both the valve frame 316 and the leaflet engagement frame 370 foreshorten in the axial direction, such that the outflow end of the leaflet engagement frame 370, defined by the engagement frame first rung 376 with the distally extending angled spikes 386, is approximated distally toward the inflow end of the leaflet engagement frame 370, which is defined by the engagement frame second rung 378^{a} in the case of leaflet engagement frame 370^{a}. The proximally extending angled spikes 388 serve to provide a counter-force that retains the inflow end of the leaflet engagement frame 370 in a relatively constant position, allowing the distally extending angled spikes 386 to move distally and drag the upper portions of the native or host leaflets therewith, thereby folding them distally/downward and away from the ostia of the coronary arteries 60, 62.

Once a selected diameter of the valve assembly 300 is reached, the actuation assemblies 40 can be uncoupled from the actuators 360 and the delivery apparatus 12^{b} can then be withdrawn from the patient's body, leaving the valve assembly 300 within the aortic annulus 52 to regulate blood flow from the left ventricle 50 into the aorta 54 as shown in FIG. 21C. As further shown in FIG. 21C, the native or host leaflet around the implanted valve assembly 300 remain folded to avoid obstruction of the ostia of the coronary arteries 60, 62.

In some designs of a prosthetic valve 310 as illustrated throughout FIGS. 17A-19, the second cells 340 can be relatively larger than cells of certain designs of balloon expandable valves or other types of prosthetic valves, such as prosthetic valve 110 illustrated in FIGS. 1A-B. In such designs, leaflet engagement frames 370 defining engagement frame cells 372 that are aligned with the second cells 340 of the valve frame 316 may experience a larger foreshortening during valve expansion, advantageously approximating the distally extending angled spikes 386 and the proximally extending angled spikes 388 toward each other along a greater axial distance, thereby potentially resulting in the native or host leaflets being folded across a longer path, distancing them further away from the ostia of the coronary arteries.

FIG. 22 shows another example of a leaflet engagement frame 370^{b} which is somewhat similar leaflet engagement frame 170^{a} described above, including only a single rung struts, namely an engagement frame first rung 376^{b} comprising distally extending angled spikes 386, and is devoid of proximally extending angled spikes 388.

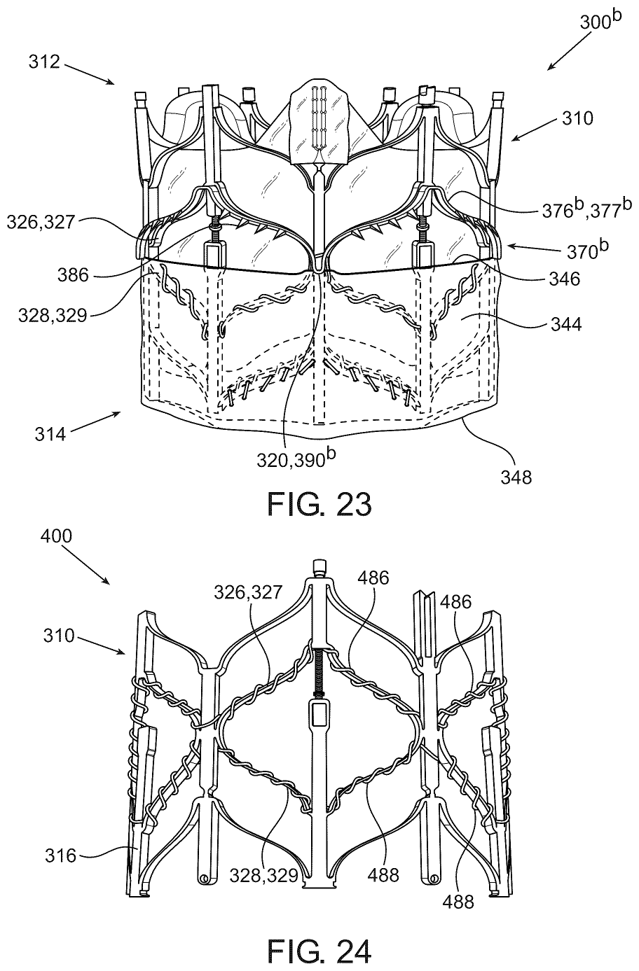

FIG. 23 shows an example of a valve assembly 300^{b} comprising the leaflet engagement frame 370^{b} coupled to the valve frame 316, such that the first curved struts 377^{b} of the engagement frame first rung 376^{b} are aligned with the curved struts 327 of the valve frame first intermediate rung 326. In some examples, as further shown in FIG. 23, the outer skirt outflow end 346 is distal to the distally extending angled spikes 386, while the outer skirt inflow end 348 can extend all the way toward the valve inflow end 314.

In the absence of proximally extending angled spikes 388, the delivery apparatus may be utilized to assist in proper positioning and folding of the leaflets in a similar manner to that described in conjunction with FIGS. 15A-D, mutatis mutandis. The valve assembly 300^{b} can be carried by the delivery apparatus 12^{b} toward the site of implantation in the same manner described above in conjunction with FIGS. 21A-C. When the valve assembly 300^{b} is disposed in or adjacent the aortic annulus 52, the actuation assemblies 40 can be utilized to radially expand the prosthetic valve 310, along with the leaflet engagement frame 370^{b} coupled thereto, from the crimped state.

Once the distally extending angled spikes 386 are engaged with the leaflets, the delivery apparatus 12^{b} is utilized to push the valve assembly 300^{b} distally (for example, toward the left ventricle 50), in this partially expanded state of the valve assembly 300^{b}. This distal displacement of the entire valve assembly 300^{b} carries the inflow ends of the native leaflets therewith in the same direction, away from the ostia of the coronary arteries 60, 62. Once the native or host leaflets are positioned as desired, the valve assembly 300^{b} can be further expanded to its final functional diameter (for example, by applying further rotational movement to the threaded rods 362 via the drivers 42), after which the actuation assemblies 40 can be uncoupled from the actuators 360 and the delivery apparatus 12^{b} can then be withdrawn from the patient's body, leaving the valve assembly 300^{b} implanted in position with the native leaflets folded away from the ostia of the coronary arteries.

In some examples, there is provided a valve assembly 400 that includes at least one proximal leaflet engagement wire 486 wrapped around a single rung of a prosthetic valve, configured to engage an upper portion of native or host leaflets, instead of a leaflet engagement frame. In some examples, the valve assembly 400 further comprises at least one distal leaflet engagement wire 488 wrapped around another single rung of the prosthetic valve, which is distal to the rung with the proximal leaflet engagement wire.

FIG. 24 shows an example of a valve assembly 400 comprising a prosthetic valve 310 with at least one proximal leaflet engagement wire 486 wrapped around the curved struts 327 of the valve frame first intermediate rung 326, and least one distal leaflet engagement wire 488 wrapped around the curved struts 329 of the valve frame second intermediate rung 328. For simplicity, soft components of the valve assembly 400, such as leaflets or skirts, are not shown in FIG. 24.

In some examples, the valve assembly 400 can comprise a single proximal leaflet engagement wire 486 wrapped around an entire rung, such as the valve frame first intermediate rung 326, in a continuous manner. In other examples, a valve assembly can comprise a plurality of proximal leaflet engagement wires 486, each wrapped around one or more sections of the rung, such as a plurality of wires 486 each wrapped around a separate curved strut 327 of the first intermediate rung 326.

In some examples, the valve assembly 400 can comprise a single distal leaflet engagement wire 488 wrapped around an entire rung, such as the frame second intermediate rung 328, in a continuous manner. In other examples, a valve assembly can comprise a plurality of distal leaflet engagement wires 488, each wrapped around one or more sections of the rung, such as a plurality of wires 488 each wrapped around a separate curved strut 327 of the first intermediate rung 326.

The wraps of either the proximal leaflet engagement wire 486 and/or the distal leaflet engagement wire 488 can be spaced from each other along the curved struts, so as to form grooves therebetween. Thus, when the valve assembly 400 is radially expanded, the native or host leaflets can extend radially into the grooves to increase engagement contact therebetween.

The proximal leaflet engagement wires 486 are configured to engage the surrounding native or host leaflets when the valve assembly 400 is partially expanded there-against, and retain engagement with the leaflets during further expansion of the valve assembly 400 such that the engaged portions of the native or host leaflets (e.g., outflow portions) will not slip over the valve assembly 400, but will rather be dragged along with the engagement wires 486 during foreshortening of the valve assembly 400, allowing the leaflets to be folded away from the ostia of the coronary arteries. Thus, the proximal leaflet engagement wires 486 are not merely designed as friction elements that may contact surrounding tissue in a manner that provides sufficient retaining force of the valve in its final expanded state after implantation, but a rather significantly greater frictional interaction that will drag outflow portions of surrounding leaflets in an axial direction during radial expansion of the valve assembly.

The distal leaflet engagement wires 488, if present, are configured to engage the native annulus or the base of the native or host leaflets, and retain this engagement in a manner that will prevent the inflow portion of the valve assembly 400 from axially slipping away from these engaged base portions, while the outflow portions of the leaflets are folded against these base portions during valve assembly radial expansion.

Various aspects of any of the proximal leaflet engagement wires 486 and/or the distal leaflet engagement wires 488 can be altered to help ensure sufficient engagement with the native or host leaflets during radial expansion of the valve assembly 400. For example, various types of materials, rigidities, widths, thicknesses, as well as wrapping configurations, including the amount and density of wraps and the amount and location of cells or strut sections to be wrapped, can be chosen.

In some examples, the proximal leaflet engagement wires 486 and/or the distal leaflet engagement wires 488 can be made of a metal wire or cable (e.g., MP35N, stainless steel, Nitinol, etc.) and/or polymeric materials. In some examples, the proximal leaflet engagement wires 486 and/or the distal leaflet engagement wires 488 can be textured along their outer surface so as to increase friction-engagement with the surrounding tissue.

Since the proximal leaflet engagement wires 486 and the distal leaflet engagement wires 488 need to fulfill different roles, as described above (i.e., wires 486 need to drag outflow portions of the leaflets in an axial direction, while wires 488 need to prevent the valve assembly 400 from axially slipping away), each can be made of different materials, and/or have different shapes or dimensions (e.g., different textures), and/or each can be wrapped around the respective struts in a different configuration (e.g., different wrap densities and the like).

In some examples, the outer skirt 344 is disposed around the outer surface of the valve frame 316 such that the outer skirt outflow end 346 is distal to both the proximal leaflet engagement wires 486 and the distal leaflet engagement wires 488, while the outer skirt inflow end 348 can extend all the way to the valve inflow end 314.

Valve assembly 400 can be utilized to fold native or host leaflets during an implantation procedure by following the steps described in conjunction with FIGS. 21A-C, for example, mutatis mutandis.

While the valve assembly 400 illustrated in FIG. 24 includes both proximal leaflet engagement wires 486 and the distal leaflet engagement wires 488, in alternative examples, a valve assembly can include only proximal leaflet engagement wires 486 wrapped around a single rung of struts of the valve frame (e.g., the valve frame first intermediate rung 326), and is devoid of distal leaflet engagement wires 488 or any other wires wrapped around any other rungs of struts. In such examples, the outer skirt outflow end is distal to the proximal leaflet engagement wires 486, while the outer skirt inflow end can extend all the way toward the valve frame inflow end. In the absence of distal leaflet engagement wires, the delivery apparatus may be utilized to assist in proper positioning and folding of the leaflets in a similar manner to that described in above for valve assembly 300^{b}, mutatis mutandis.

While the valve assembly 400 illustrated in FIG. 24 shows proximal leaflet engagement wires 486 and distal leaflet engagement wires 488 wrapped around struts of a mechanically expandable prosthetic valve 310, it is to be understood that proximal leaflet engagement wires 486, and optionally distal leaflet engagement wires 488, can be similarly used in combination with other types of prosthetic valves, such as prosthetic valve 110. In such examples (not shown), the proximal leaflet engagement wires 486 can be wrapped, for example, around angled struts 127 of the first intermediate rung 126. Additionally, distal leaflet engagement wires 488 can be optionally wrapped around the struts of one other rung, distal to the proximal leaflet engagement wires 486. In one example, distal leaflet engagement wires 488 are wrapped around the angled struts 133 of the valve frame inflow rung 132. In another example, distal leaflet engagement wires 488 are wrapped around the angled struts 131 of the valve frame third intermediate rung 130. In yet another example, distal leaflet engagement wires 488 are wrapped around the angled struts 129 of the valve frame second intermediate rung 128. In any such configurations, the outer skirt outflow end 146 is distal to the proximal leaflet engagement wires 486.

In some examples, there is provided a mechanically expandable prosthetic valve 510 comprising distally extending angled spikes 586, and optionally proximally extending angled spikes 588, which are integrally formed with the valve frame 516 end extend directly from angled struts of the prosthetic valve 510, such that the prosthetic valve can be utilized in a similar manner to that described above for valve assemblies 300, but without being coupled to a leaflet engagement frame. The prosthetic valve 510 may be similar in other respects to prosthetic valve 310, with similar reference numerals referring to similar features, and in the interest of brevity will not be described further.

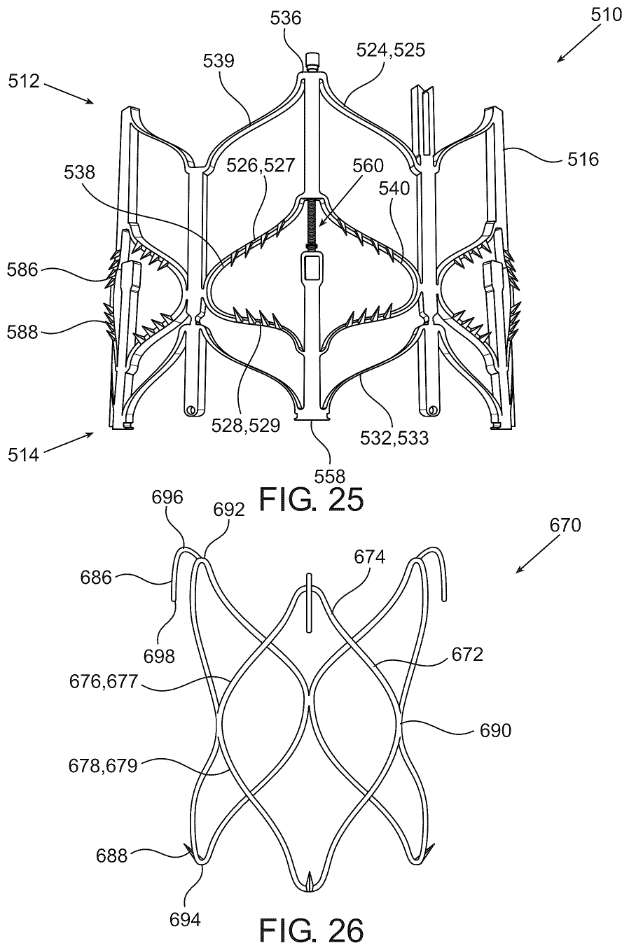

FIG. 25 shows an example of a prosthetic valve 510, which can be similar to prosthetic valve 310, except that it comprises a plurality of distally extending angled spikes 586 extending from curved struts 527 of a valve frame first intermediate rung 526, and a plurality of proximally extending angled spikes 588 extending from curved struts 529 of a valve frame second intermediate rung 528. For simplicity, soft components of prosthetic valve 510, such as leaflets or skirts, are not shown.

The distally extending angled spikes 586 may be implemented to extend from the valve frame first intermediate rung 526 in the same manner described for any example of the distally extending angled spikes 386 extending from the first rung 376^{a} of leaflet engagement frame 170^{a}. The proximally extending angled spikes 588 may be implemented to extend from the valve frame second intermediate rung 528 in the same manner described for any example of the proximally extending angled spikes 388 extending from the second rung 378^{a} of leaflet engagement frame 370^{a}. An outer skirt 544 can extend around the outer surface of the valve frame 516 such that the outer skirt outflow end 546 is distal to the distally extending angled spikes 586 and the proximally extending angled spikes 588 (outer skirt 544 and outer skirt outflow end 546 are not shown in FIG. 25, but can be functionally similar to outer skirt 144 and outer skirt outflow end 146, respectively, shown in FIG. 1A).

The prosthetic valve 510 can be delivered by a delivery apparatus 12^{v} and implanted following the same steps described hereinabove in conjunctions with FIGS. 21A-C, mutatis mutandis, so as to fold the native or host leaflets to prevent obstruction of the ostia of the coronary arteries.

In another example, a prosthetic valve 510 can include distally extending angled spikes 586 extending from the valve frame first intermediate rung 526, while the valve frame second intermediate rung 528 (or any other rung of the valve frame 516) remain devoid of extending angled spikes (or any other spikes). In such examples (not shown), the outer skirt outflow end 546 can be distal to the distally extending angled spikes 586. This type of a prosthetic valve can be delivered by a delivery apparatus 12^{b} and implanted following the same steps described hereinabove for valve assembly 310^{b}, mutatis mutandis, so as to fold the native or host leaflets to prevent obstruction of the ostia of the coronary arteries.

The examples of prosthetic valve 310 illustrated throughout FIGS. 17A-23 show second cells 340 positioned within first cells 339 at the middle between the valve outflow end 312 and the valve inflow end 314. In some examples, the curvature of curved struts 327 of the first intermediate rung 326 is identical to the curvature of the corresponding curved struts 325 of the outflow rung 324, and the curvature of curved struts 329 of the second intermediate rung 328 is identical to the curvature of the corresponding curved struts 333 of the inflow rung 332. In some examples, the axial distance between each curved strut 327 of the first intermediate rung 326 and the respective curved strut 325 of the outflow rung 324, at any specific circumferential location around the valve frame 316, is identical to the axial distance between the curved strut 329 of the second intermediate rung 328 and the respective curved strut 333 of the inflow rung 332 at the same circumferential location.

In contrast, the example of prosthetic valve 510 illustrated in FIG. 25 shows second cells 540 that are generally closer to the valve inflow end 514 than the valve outflow end 512. In some examples, the curvature of curved struts 527 of the first intermediate rung 526 is different than the curvature of the corresponding curved struts 525 of the outflow rung 524, and the curvature of curved struts 529 of the second intermediate rung 528 is different than the curvature of the corresponding curved struts 533 of the inflow rung 532. In some examples, the axial distance between each curved strut 527 of the first intermediate rung 526 and the respective curved strut 525 of the outflow rung 524, at any specific circumferential location around the valve frame 516, is greater than the axial distance between the curved strut 529 of the second intermediate rung 528 and the respective curved strut 533 of the inflow rung 532 at the same circumferential location. For example, in the illustrated implementation, the junctions at which each couple of curved struts 527 and 529 converge with axial support posts 334, are in close proximity to the junctions at which the curved struts 333 of the inflow rung 332 converge with the same axial support posts 334.

In some cases, the size of the prosthetic valve, such as prosthetic valve 310, and its position within the native anatomy once implanted, places the valve frame first intermediate rung 326 too proximal relative to the outflow region of the native or host leaflets, which may result in misappropriate engagement with these regions of the leaflets. In such cases, lowering the second cells to a position which is closer to the valve inflow end, as shown for example for second cells 540 of prosthetic valve 510, may be advantageous to ensure proper engagement with the native or host leaflets for folding them during radial expansion.

Thus, in some examples, the second cells 340 of prosthetic valve 310 utilized in any of the valve assemblies 300 or 400, can be positioned closer to the inflow end of the valve as described above in conjunction with FIG. 25. Similarly, it is to be understood that the lower position of second cells 540 illustrated in FIG. 23 is not binding, and that in other examples, a prosthetic valve 510 can include second cells that are positioned in the middle of the first cells, equally distanced from both the inflow and outflow ends, as described above for second cells 340 in conjunction with FIGS. 17A-23.

Another important factor in prosthetic valve implantation procedures is properly positioning the prosthetic valve within the heart valve annulus. Most conventional implantation procedures use fluoroscopy and/or echography to properly position the prosthetic valve within the native valve annulus prior to deployment. Such imaging modalities involve extensive and complicated equipment, and may also have limitations in their accuracy in some circumstances.

According to some examples, any of the valve assemblies that include leaflet engagement frames with distally extending angled spikes, such as valve assemblies 100, 300, 400, as well as any prosthetic valves that include integrally formed distally extending angled spikes, such as prosthetic valve 210 or 510, can be utilized to provide feedback to a user to confirm proper positioning of the prosthetic valve with respect to the native annulus.

For example, a valve assembly or a prosthetic valve can be carried by a delivery apparatus 12 to an implantation site, and partially expanded, utilizing any of the methods described above (including inflating a balloon 26 of delivery apparatus 12^{a} or mechanical expansion via actuation assemblies 40 of delivery apparatus 12^{b}), until the spikes, that include any of the distally extending angled spikes 186, 286, 386, 586 and when present, may also include any of the respective proximally extending angled spikes 188, 288, 388, 588, engage (i.e., contact, with or without penetration into the surrounding tissue) the native or host leaflets. In some instances, the spikes can engage calcified regions of native leaflets.

Using tactile feedback created by the engagement of the spikes with the native or host leaflets, the user can confirm the proper positioning of the prosthetic valve. With the proper positioning confirmed, the user can further inflate the balloon or operate the actuation assemblies, depending on the type of delivery apparatus and prosthetic valve in use, to effectuate further expansion of the prosthetic valve into the desired position within the native valve annulus (or within the previously implanted valve for ViV procedures).

In some implementations, the user may rely on additional positioning techniques, such as fluoroscopy, echocardiography, etc. For example, during initial advancement of the delivery assembly into the heart, the user may use fluoroscopic, echocardiagraphic, and/or other imaging methods to provide visual confirmation of the orientation and position of the catheter, prosthetic valve, and/or positioning elements relative to the native valve annulus or other deployment site. The user may also use the fluoroscopic, echocardiagraphic, and/or other imaging methods to provide visual confirmation of the orientation and position of various elements of the delivery system in addition to the tactile feedback provided by the positioning elements, e.g., during the positioning of the prosthetic valve described above using tactile feedback from the spikes. The tactile feedback thus provides the user with another important sensory cue to the relative position of the spikes/prosthetic valve with respect to the native valve annulus.

In some implementation, any of the distally extending spikes and/or proximally extending spikes of any of the prosthetic valves and/.or prosthetic assemblies disclosed hereinabove, are not necessarily angled, meaning that the angle α and/or the angle β is 0 degrees, such that the corresponding spikes are flush with the annular surface 175. In cases in which engagement between such non-angled spikes and the surrounding anatomy (e.g., native or host leaflets in the case of distally extending spikes, or base of the leaflets or native annulus for proximally extending spikes) is sufficient to facilitate leaflet folding according to any of the methods described above, such configurations may have the advantage of being produced via simpler manufacturing techniques, such as laser cutting the struts and non-angled spikes extending therefrom, from a cylindrical piece of metal (or any other relevant material).

Thus, any of the valve assemblies 100 (including valve assemblies 100^{a}, 100^{b}, 100^{c}) or 300 (including valve assemblies 300^{a}, 300^{b}), and any of the prosthetic valves 210 (including prosthetic valves 210^{a}, 210^{b}, 210^{c}) or 510, can include distally extending spikes that can be either angled or non-angled, as well as proximally extending spikes that can be angled or non-angled. Except for the spikes being optionally non-angled, any of such valve assemblies or prosthetic valves will be similar in all other respects to any of the examples described hereinabove, and in the sake of brevity will not be described further.

Any reference to a rung of struts throughout the current specification, including any inflow rung, outflow rung, or intermediate rung of any of the prosthetic valves and/or leaflet engagement frames described herein, refers to a row that includes a series of interconnected struts which can be angled struts or curved struts, but not axial struts.

In some examples, a valve assembly 600 comprises a prosthetic valve and leaflet engagement frame that includes exactly three cells and three distally extending hooks at its outflow apices. In some examples, the leaflet engagement frame further includes three proximally extending angled spikes at its inflow apices.

FIG. 26 shows a leaflet engagement frame 670, according to some examples. The leaflet engagement frame 670 comprises three enlarged engagement frame cells 672 defined by intersecting engagement frame struts 674, such as first struts 677 of engagement frame first rung 676 and second struts 679 of engagement frame second rung 678. The first struts 677 and the second struts 679 can be either relatively straight angled struts or curved struts. Each engagement frame cell 672 extends between an engagement frame outflow apex 692 defined by the convergence of upper ends of two first struts 677 and an engagement frame inflow apex 694 defined by the convergence of lower ends of two second struts 679. The three cells 672 are connected to each other at engagement frame intermediate junctions 690, defined by the convergence of lower ends of first struts 677 and upper ends of second struts 679.

As further shown in FIG. 26, the leaflet engagement frame 670 comprises three distally extending hooks 686 extending from the engagement frame outflow apices 692. Each distally extending hook can include an inverse U-shaped curved portion 696 connected on one end to a corresponding outflow apex 692, and an opposite free end 698 positioned radially away from the corresponding outflow apex 692 and oriented distally/downward. The distally extending hooks 686 are configured to capture in the curved portions 696 proximal ends of one or more native or host leaflets, when the valve assembly 600 is expanded to a partially expanded state, and to distally fold the captured native or host leaflets when the valve assembly 600 is further expanded.

In some examples, as also shown in FIG. 26, the leaflet engagement frame 670 comprises proximally extending angled spikes 688 extending proximally and radially away from the engagement frame inflow apices 694. The overall characteristics, including the orientation of the engagement frame inflow apices 694, can be similar to the examples described above for proximally extending angled spikes 188. However, since the leaflet engagement frame 670 includes only three proximally extending angled spikes 688 extending from three engagement frame outflow apices 692, compared to optionally a significantly greater number of proximally extending angled spikes 188 that can extend from struts of leaflet engagement frame 170, the proximally extending angled spikes 688 may be longer, thicker and/or sharper than the plurality of proximally extending angled spikes 188 so as to achieve a similar functionality of retaining the leaflet engagement frame 670 in position against the native annulus or base portion of native or host leaflets.

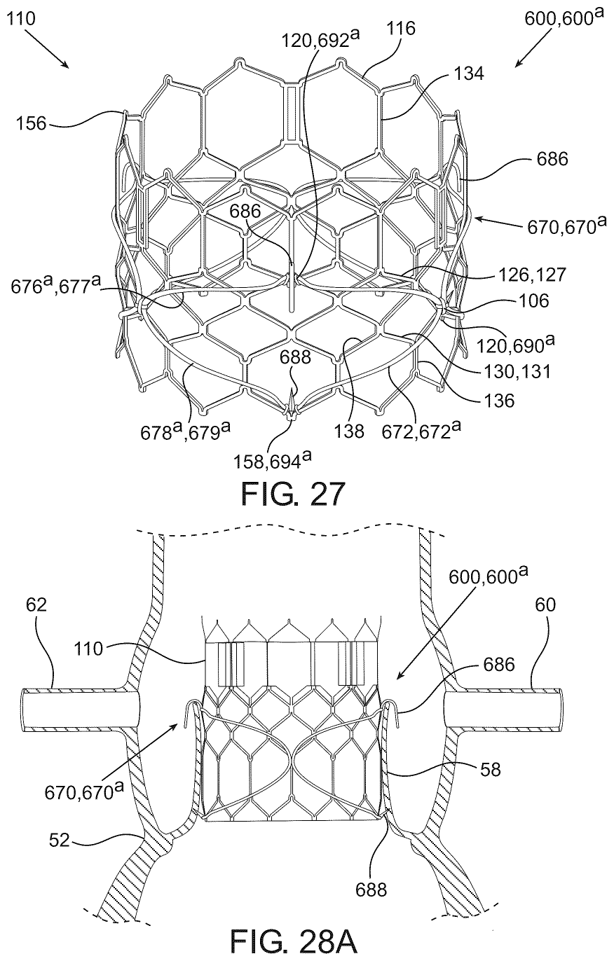

FIG. 27 shows one example of valve assembly 600^{a} that comprises a prosthetic valve 110 as described above, and a leaflet engagement frame 670^{a} coupled thereto. As shown, each row of cells 138 of the prosthetic valve 110 can include a greater number of cells, such as twelve cell in the illustrated example, compared to the three cells 672^{a} of the leaflet engagement frame 670^{a}. For simplicity, soft components of the valve assembly 600^{a}, such as leaflets and skirts, are not shown.

In some examples, a valve assembly 600 comprises a leaflet engagement frame 670 that includes three engagement frame cells 672, and a prosthetic valve that includes at least one row along a region to which the leaflet engagement frame 670 is coupled, with a greater number of cells. According to some examples, the valve assembly 600 comprises a prosthetic valve that includes at least one row with twelve cells (such as in the example illustrated in FIG. 27). According to some examples, the valve assembly 600 comprises a prosthetic valve that includes at least one row with nine cells (not shown). According to some examples, the valve assembly 600 comprises a prosthetic valve that includes at least one row with six cells (such as in the example illustrated in FIG. 29).

Unlike the case with valve assemblies 100 or 300, for example, the engagement frame struts 174 are not necessarily aligned with struts of the prosthetic frame due to the different number and size of the cells and struts of each frame. In some examples, a valve assembly 600 can comprise a prosthetic valve that also includes at least one row of three cells (examples not shown). Such examples can include frame cells that are similarly sized and shaped as those of the leaflet engagement frame 670, such that the engagement frame struts 674 can be aligned with struts of the valve frame. Alternatively, such examples can include frame cells that have different size or shape than the cells of the leaflet engagement frame 670, such that even though each frame includes a row of three cells, the struts of each frame are not necessarily aligned.

The leaflet engagement frame 170 can be coupled to the valve frame with sutures or other fasteners. In some examples, the engagement frame outflow apices 692, engagement frame inflow apices 694, and engagement frame intermediate junctions 690 are coupled to junctions of the prosthetic valve (including, optionally, to apices of the prosthetic valve), wherein such coupling can be achieved according to any example described above for coupling engagement frame junctions 190 to valve frame junctions 120.

In the example illustrated in FIG. 27, the engagement frame outflow apices 692^{a} are coupled to valve frame junctions 120 defined by the convergence of upper ends of angled struts 127 of the valve frame first intermediate rung 126, the engagement frame intermediate junctions 690^{a} are coupled to valve frame junctions 120 defined by the convergence of upper ends of angled struts 131 of the valve frame third intermediate rung 130, and the engagement frame inflow apices 694^{a} are coupled to inflow apices 158 of the valve frame 116. In general, the position of the engagement frame outflow apices 692 over the frame of the prosthetic valve can be chosen so as to place the distally extending hooks 686 that extend from the engagement frame outflow apices 692, at a level that will properly engage and fold native or host leaflets during radial expansion.

In some examples, the length of the first struts 677 of the engagement frame first rung 676 and the length of the second struts 679 of the engagement frame second rung 678 are not identical. For example, the second struts 679^{a} illustrated in FIG. 27 are longer than the first struts 677^{a}. This non-symmetrical shape of engagement frame cells 672^{a} may be required when used in combination with a prosthetic valve 110 equipped with a lower row of cells that are higher than the cells of the intermediate rows of the valve frame 116.

FIGS. 28A-B show stages of an exemplary method for folding native or host leaflets during implantation of a valve assembly 600, such as the valve assembly 600^{a}. The valve assembly 600 can be carried over the delivery apparatus 12 toward the site of implantation in the same manner described above in conjunction with FIGS. 9A-D. For simplicity, The delivery apparatus 12, including an inflatable balloon, are not shown. FIG. 28A shows the valve assembly 600 in a partially expanded state, such that the distally extending hooks 686 contact the proximal ends of the native or host leaflets, and the proximally extending angled spikes 688 engage with (for example, penetrate into) the base of the native or host leaflets (or the aortic annulus).

Further expansion of the valve assembly 600 to the functional size of the prosthetic valve 110 serves to fold the native or host leaflets. FIG. 28B shows the fully expanded valve assembly 600. During this expansion to the prosthetic valve's functional diameter, both the valve frame 116 and the leaflet engagement frame 670 foreshorten in the axial direction, such that the engagement frame outflow apices 692 move distally toward the engagement frame inflow apices 694.

The proximally extending angled spikes 688 are held in an axial position relative to the aortic annulus 52 (or relative to the scalloped line along which host leaflets are attached to the frame of a previously implanted valve, for ViV procedures) during radial expansion of the valve assembly 600, which allows the distally extending hooks 686 to move distally and drag the proximal ends of the native or host leaflets therewith, thereby folding them distally/downward and away from the ostia of the coronary arteries 60, 62.

Once a selected diameter of the valve assembly 600 is reached, the delivery apparatus 12 can be decoupled and withdrawn from the patient's body, leaving the valve assembly 600 within the aortic annulus 52 to regulate blood flow from the left ventricle 50 into the aorta 54. The native or host leaflet around the implanted valve assembly 600 are retained distal of the ostia of the coronary arteries 60, 62 with the distally extending hooks 686, thus reducing the possibility of the native leaflets 58 fully or partially covering the coronary ostia. The native leaflets 58 may have been displaced distal of the proximal ends of the valve leaflets 152. Upon implantation, the valve leaflets 152 operate as prosthetic replacements for the native leaflets 58.

In some examples, a leaflet engagement frame 670 can include only the distally extending hooks 686, without any proximally extending angled spikes (example not shown). In the absence of proximally extending angled spikes 688, the delivery apparatus may be utilized to assist in proper positioning and folding of the leaflets in a similar manner to that described in conjunction with FIGS. 15A-D, mutatis mutandis. The valve assembly 600 can be carried by the delivery apparatus 12 toward the site of implantation and partially expanded in the same manner described above in conjunction with FIG. 28A.

Once the distally extending hooks 686 are in contact with the proximal ends of the leaflets, the delivery apparatus 12 can be utilized to push the valve assembly 600 distally in this partially expanded state of the valve assembly. This distal displacement of the entire valve assembly 600 carries the proximal ends of the native leaflets therewith in the same direction, away from the ostia of the coronary arteries 60, 62. Once the native or host leaflets are positioned as desired, the valve assembly 600 can be further expanded to its final functional diameter, after which the delivery apparatus 12 can be decoupled and withdrawn from the patient's body, leaving the valve assembly 600 implanted in position with the native leaflets folded away from the ostia of the coronary arteries.

FIG. 29 shows another example of valve assembly 600^{b} that comprises a mechanically expandable prosthetic valve 310 as described above, and a leaflet engagement frame 670^{b} coupled thereto. As shown, prosthetic valve 310 can include a row of six second cells 340 contained within a row of six first cells 339, while the leaflet engagement frame 670^{b} includes three cells. For simplicity, soft components of the valve assembly 600^{b}, such as leaflets and skirts, are not shown.

In this example, the engagement frame outflow apices 692^{b} are coupled to the valve frame at a junction defined the convergence of lower ends of curved struts 325 of the valve frame inflow rung 332 with an axial support post 334, the engagement frame intermediate junctions 690^{b} are coupled to at a junction defined the convergence of lower ends of curved struts 327 of the valve frame first intermediate rung 326 with axial support posts 334, and the engagement frame inflow apices 694^{a} are coupled to the valve frame at a junction defined the convergence of upper ends of curved struts 333 of the valve frame inflow rung 332 with an axial support post 334. Unlike the example of leaflet engagement frame 670^{a}, the lengths of first struts 677^{b} of the engagement frame first rung 676^{b} are equal to the lengths of the second struts 679^{b} of the engagement frame second rung 678^{b}.

Implantation of the valve assembly 600^{b} can be similar to that described above in conjunction with FIG. 28A-B, with the exception that a delivery apparatus 12^{b} can be utilized during the procedure in the same manner described above in conjunction with FIGS. 21A-C, mutatis mutandis. Similarly, while illustrated with proximally extending angled spikes 688^{b} in FIG. 29, it is to be understood that the valve assembly 600^{b} can comprise a leaflet engagement frame 670^{b} that is devoid of proximally extending angled spikes, which can be similarly implanted by utilizing the delivery apparatus to assist in proper positioning and folding of the leaflets as described above, mutatis mutandis.

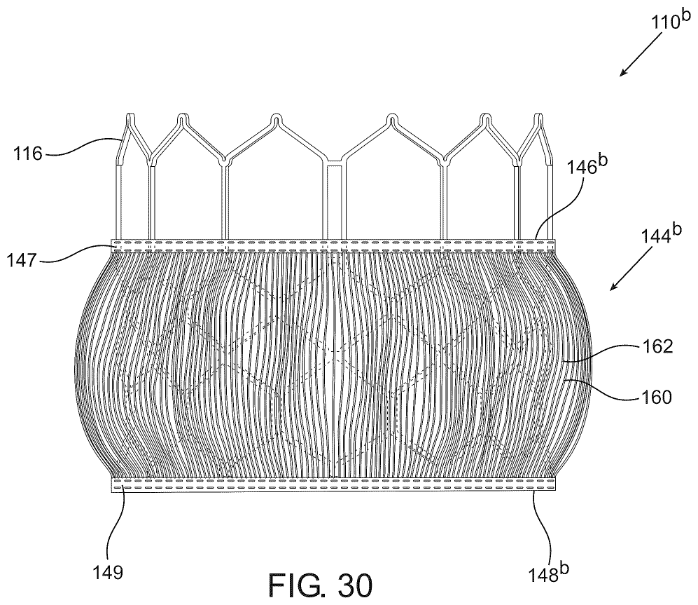

Some types of outer skirts of prosthetic valves can include a plurality of yarns or fibers that can extend radially outward from the frame to contact the surrounding tissue, configured to increase the surface area available for thrombogenesis and tissue growth. FIG. 30 shows an exemplary prosthetic valve 110^{b}, which can be similar to prosthetic valve 110^{a} described above, with the exception that it includes an outer skirt 144^{b} equipped with a floating fiber portion 160 extending between the outer skirt outflow end 146^{b} and the outer skirt inflow end 148^{b}. In some implementations, the outer skirt 140^{b} includes an outflow woven portion 147 and an inflow woven portion 149 extending from the outer skirt outflow end 146^{b} and the outer skirt inflow end 148^{b}, respectively. The floating fiber portion 160 can be disposed between the outflow woven portion 147 and the inflow woven portion 149, such that it is bounded or edged in a direction parallel to the longitudinal axis by the outflow and inflow woven portions 147 and 149. The floating fiber portion 160 can comprise a plurality of filaments 162 that curve radially outwards, forming a “puffy” configuration. In some implementations, the filaments can be texturized filaments 162.

The filaments 162 can extend between the outflow and inflow woven portions 147, 149. In the illustrated example, the filaments 162 extend in only one direction (e.g., parallel to the longitudinal axis of the prosthetic valve 110). However, in other examples, the filaments 162 can extend between the outflow and inflow woven portions at an angle. In some examples, various filaments 162 can extend in any of various directions (for example, in varying angles). In some examples, different filaments 162 can extend in different angles. For example, one group of filaments 162 can extend in a first direction, while at least one other group of filaments can extend in a second direction, which is different from the first directions. Any number of filaments or groups of filaments can extend in any number of differently oriented directions relative to each other. In some examples, the floating fiber portion 160 can include multiple layers of filaments 162 (e.g., on top of each other).

In certain examples, the floating fiber portion 160 can be resiliently stretchable between a first, natural, or relaxed configuration corresponding to the radially expanded state of the prosthetic valve, and a second, elongated, or tensioned configuration corresponding to the radially compressed state of the prosthetic valve. In some examples, additionally or alternatively, the filaments 162 can be sized such that they abut the valve frame 116 or a base layer of the outer skirt disposed around the valve frame, when the prosthetic valve 110^{b} is in a crimped or radially compressed configuration, and such that they curve outwards to extend radially away from the valve frame 116 when the prosthetic valve 110^{b} is in the expanded configuration (e.g., to create a “puffy” configuration). This configuration can promote tissue ingrowth around the prosthetic valve 110, which can advantageously result in decreased PVL. Further details regarding various weave patterns and techniques for creating floating fiber portions are disclosed in U.S. Publication 2019/0192296, which is incorporated herein by reference in its entirety.

The fibers 162 can comprise any of various biocompatible thermoplastic polymers such as PET, Nylon, ePTFE, UHMWPE, etc., or other suitable natural or synthetic fibers. In certain examples, the floating fiber portion 160 can be woven on a loom, and can then be heat-treated or heat-set to achieve the desired size and configuration. For example, depending upon the material selected, heat-setting can cause the portion 160. Heat-setting can also cause a texturizing effect, or increase the amount of texturizing, of the texturized filaments 162. Heat setting can also induce thrombogenic characteristics to the polymer surface, which may be beneficial for PVL sealing.

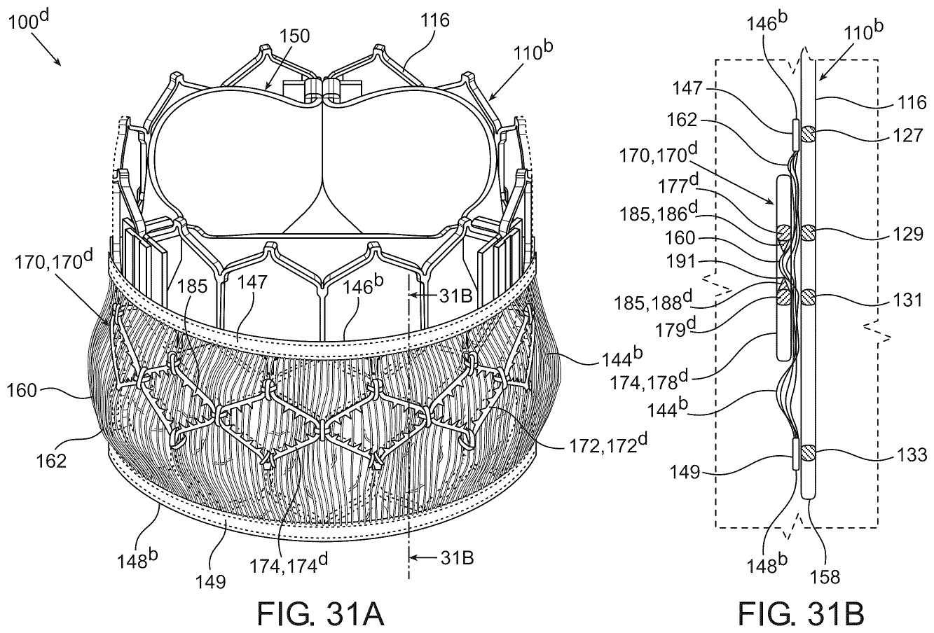

FIG. 31A shows an example of a valve assembly 100^{d} comprising a leaflet engagement frame 170 coupled to and disposed around a floating fiber portion 160 of an outer skirt 146^{b} of a prosthetic valve 110^{b}. FIG. 31B shows a cross-sectional view along line 31B-31B of FIG. 31A. A specific type of a leaflet engagement frame 170^{d} is illustrated in FIGS. 31A-31B, though any other type of a leaflets engagement frame can be utilized. A leaflet engagement frame 170 attached to a floating fiber portion 160 can include at least one rung of engagement frame struts 174, from which a plurality of spikes 185 terminating with sharp tips 191 extend.

In the illustrated example, a leaflet engagement frame 170^{d} is shown to include two rungs of struts, such as engagement frame first rung 176^{d} and engagement frame second rung 178^{d} defining a single row of engagement frame cells 172^{d}, in a similar configuration to that described above for leaflet engagement frame 170^{b}. The main difference is that the spikes 185^{d}, which can include both distally extending spikes and proximally extending spikes, are not necessarily angled, but can be rather formed to extend along the same plane (e.g., circumferential plane) defined by the engagement frame struts 174^{d}, meaning that the tips 191 of the spikes 185 can lie along the same plane of the engagement frame struts 174^{d}, instead of extending radially away from the valve frame 116, for example. Nevertheless, in other examples, the spikes 185 of a leaflet engagement frame 170 disposed around a floating fiber portion 160 can be angled in the same manner described above with respect to leaflet engagement frame 170^{a}, 170^{b} and 170^{c}.

While the struts 185^{d} of leaflet engagement frame 170^{d} are shown to include distally extending spikes 186^{d} that extend from the engagement frame first rung 176^{d} and proximally extending spikes 188^{d} that extend from the engagement frame second rung 178^{d}, it is to be understood that, unlike the examples illustrated and described above for other leaflet engagement frames 170^{a}, 170^{b} and 170^{c}, any of the engagement frame struts 174^{d}, including first angled struts 177^{d} and second angled struts 179^{d}, can include spikes 185^{d} that extend therefrom in both directions, including proximally extending spikes 188^{d} that can optionally extend from first angled struts 177^{d} and distally extending spikes 186^{d} that can optionally extend from second angled struts 179^{d} (configuration not shown explicitly). Moreover, a leaflet engagement frame 170^{d} can include more than two rungs of engagement frame struts 174^{d}, for example such as the four rungs illustrated for exemplary leaflet engagement frame 170^{a}. In such arrangements, intermediate rungs, such as an engagement frame second rung and an engagement frame third rungs (both of which disposed between engagement ring first rung and engagement frame fourth rung), are not necessarily devoid of spikes (as is the case with exemplary leaflet engagement frame 170^{a}), but can rather also include spikes 185 that can extend from their struts in both the proximal and distal directions.

As shown, the leaflet engagement frame 170 can be disposed over the floating fiber portion 160, optionally between outer skirt outflow end 146^{b} and outflow skirt inflow end 148^{b}, and in some examples, between outflow woven portion 147 and inflow woven portion 149. Since the filaments 162 of the floating fiber portion 160 are curved radially outward in a relatively loose manner in the radially expanded configuration, portions of the filaments can be squeezed between the engagement frame struts 174 and the valve frame 116, with the remaining portion of the filaments, such as above and below the leaflet engagement frame 170, as well as portions extending through openings of engagement frame cells 172, can still bulge or curve radially outward, away from the valve frame 116 and toward the surrounding tissues. Since the filaments 162 extend in a rather loose manner along the outer skirt 144^{b}, spikes 185 that extend in any orientation, including angled or in-plane spikes, and including any orientation in a proximal or distal direction, do not pose a risk of damaging the outer skirt 144^{b} as the sharp tips 191 can pass through spaced between adjacent filaments 162.

While a valve assembly 100^{d} can be optionally utilized for engaging and lowering or folding host leaflets, as described above for any of the valve assemblies 100^{a}, 100^{b} and 100^{c}, another optional advantageous utilization of valve assembly 100^{d} is for improved anchoring against native tissue, which can be of increased importance in patients with relatively non-stenotic anatomy (such as patients suffering from chronic aortic insufficiency or aortic regurgitation) and/or at implantation locations that do not provide sufficient structure for a typical prosthetic heart valve (e.g., at a native mitral valve or a native tricuspid valve). In such cases, the spikes 185 of the leaflet engagement frame 170 can be configured to increase friction between the valve assembly 100^{d} and the native tissue, and resist migration relative to the native tissue.

As mentioned above, the filaments 162 of a floating fiber portion 160 are designed to as to increase the surface area of the outer skirt 144^{b} to improve blood clotting. Fibers from which filaments 162 are formed can be sized to promote a biological response or interaction at the cellular level between the filaments 162 and the blood flowing past the skirt 144^{b}. For example, blood cells typically range in size from 2 μm to 15 μm. For example, the diameter of red blood cells typically ranges from 6 μm to 8 μm, and the diameter of platelets typically ranges from 2 μm to 3 μm. Thus, utilizing fibers having a diameter sized to approximately match the diameter of blood cells (e.g., 1 μm to 20 μm) can promote interaction between the filaments and blood cells at the cellular level.

The outwardly curved filaments 162 can obstruct the flow of blood past the valve (for example, between the valve and surrounding tissue), reducing the velocity and volume of blood that leaks past the valve assembly 100^{d} after implantation. The flow obstruction provided by the filaments 162 can increase the dwell time of blood near the outer skirt 144^{b}. This, together with the fiber diameters described above, can induce thrombus formation and promote sealing between the skirt and the surrounding tissue.

Nevertheless, thrombus may form over a specific time period passing from the moment of implantation, during which PVL sealing may be incomplete. Thus, it may be desirable to reduce such time period to a minimum, The spikes 185 of the leaflet engagement frame 170 cause local trauma to the surrounding tissue, due to the engagement between the sharp tips 191 and the surrounding anatomy. This can promote rapid biological response, localized at the site of engagement between the spikes 185 and the surrounding tissue, which can significantly shorten the duration of thrombus formation along the filaments 162 of the outer skirt 144^{b}. Thus, the combination of the leaflet engagement frame 170 and an outer skirt 144^{b} equipped with a floating fiber portion 160, can enhance biological response and thrombus formation over the filaments 162 in a manner that rapidly increases PVL sealing.

Conventional prosthetic valves 110 may be appropriately sized for placement inside many native cardiac valves or orifices, such as within a native aortic annulus 52. However, with larger native valves (e.g., a tricuspid native valve or a mitral native valve), such conventional prosthetic valve 110 might be too small to secure into the larger annulus. In this case, the prosthetic valve may not be large enough to sufficiently expand inside and properly seal against the native the native annulus.

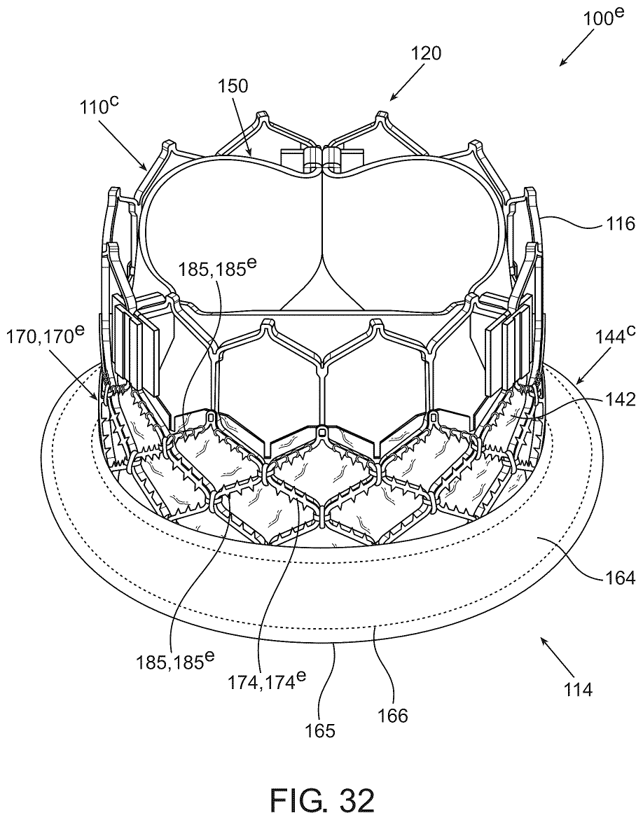

FIG. 32 shows an example of a valve assembly 100^{e} comprising a leaflet engagement frame 170 coupled to and disposed radially around the frame valve 116 of a prosthetic valve 110^{c}. Prosthetic valve 110^{c} can be similar to prosthetic valve 110^{c} described above in conjunction with FIGS. 1A-1B, except that it includes an outer skirt 144^{c} that comprises a sealing ring 164. The sealing ring 164 desirably is sized such that when the valve assembly 100 is implanted in the native annulus, it completely covers any gap that may exist between the valve frame 116 and the native annulus.

The outer skirt 144^{c} can further include a flat base layer disposed around the valve frame 116 (in a similar manner to outer skirt 144^{a} illustrated in FIG. 1A) to which the sealing ring 164 is attached, or can be comprised solely of the sealing ring 164 without including a flattened base layer. The sealing ring 164 can be coupled (e.g., sutured) to the valve frame 116 and/or to an inner skirt 142 of the prosthetic valve 110^{c}. In some implementations, as shown, the sealing ring 164 can be disposed around the valve inflow end 114.

The leaflet engagement frame 170 of valve assembly 100^{e} can formed according to any examples described above in conjunction with valve assembly 100^{d}, including by being implemented as any one of leaflet engagement frames 170^{a}, 170^{b}, 170^{c} or 170^{d}, meaning that it can include any number of rungs of engagement frame struts 174, with a plurality of spikes 185 that can be either angled or in-plane spikes, and can protrude in any direction from any of the engagement frame struts 174. In the illustrated example, a leaflet engagement frame 170^{e} is shown to include several rungs of engagement frame struts 174^{e}, wherein at least some of the engagement frame struts 174^{e}, such as the struts defining an intermediate rung in the illustrated example, can include spikes 185 extending from both sides of the struts. In other words, a leaflet engagement frame 170 comprised in valve assembly 100^{e} can optionally include both distally extending spikes 186 and proximally extending struts 188, extending from opposite edges of the same engagement frame struts 174, as illustrated for engagement frame struts 174^{e} of a middle rung of leaflet engagement frame 170^{e} in FIG. 32. The spikes can be either angled or non-angled (i.e., extending in a direction parallel to the central axis of the valve assembly).

In some implementations, when the outer skirt 144^{c} further includes a base layer to which the sealing ring 164 is attached, the leaflet engagement frame 170 can be attached to (e.g., sutured to) this base layer of the outer skirt. In alternative implementations, the leaflet engagement frame 170 can be directly attached to the valve frame 116, in the same manner described above for any of the valve assemblies 100^{a}, 100^{b}, and 100^{c}.

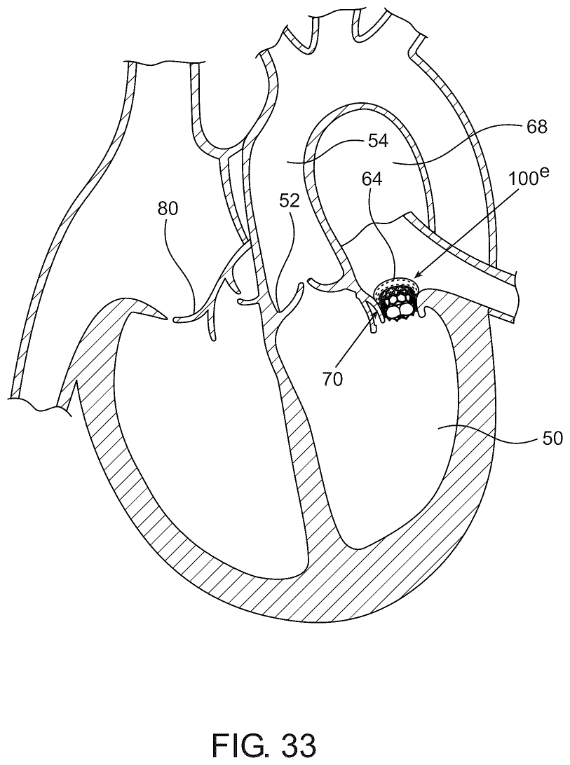

FIG. 33 depicts a valve assembly 100^{e} implanted in a native mitral valve 70. The valve assembly 100^{e} is particularly suitable for deployment in a larger native annulus, such as that of the native mitral valve, which can in some instances lack the sufficient anatomical structure to retain a typical prosthetic valve in place. This is because the leaflet engagement frame 170 disposed around the prosthetic valve 110^{c} provides increased frictional engagement with the native tissue (e.g., native mitral leaflets), thereby improving the valve assembly's ability to resist migration relative to the native tissue, even in locations like the native mitral valve where typical prosthetic valves may be unsuitable. While the leaflet engagement frame 170 serves to better retain the valve assembly 100^{e} in position, the increased diameter of the sealing ring 164 can provide adequate PVL sealing, even if the valve assembly 100^{e} is implanted in a range of native annuluses that can be greater in size than the prosthetic valve 110^{c} diameter.

In some implementation, such as in the illustrated example, the sealing ring 164 can be configured to abut the atrial side of the mitral valve 70 (i.e., the side facing the left atrium 68 in FIG. 33), in a manner that allows the sealing ring 164 to completely cover any gaps or opening between the mitral valve 70 and the valve frame 116. The sealing ring 164 is preferably impervious to the flow of blood, allowing it to effectively block blood from flowing back into the left atrium 68 between outer surfaces of the valve assemble 100^{e} and the native tissue, ensuring that all, or substantially all, of the blood passes through the leaflet assembly 150 from the left atrium 68 to the left ventricle 50. In such implementation, the sealing ring 164 can also serve to better retain the valve assembly 100^{e} in place against migration toward the left ventricle 50.

Alternatively, or additionally, the sealing ring 164 can be made of a relatively compressible or squeezable material, configured to have at least a portion thereof squeezed within the native annulus (e.g., the annulus of the mitral valve), such that the ring's 164 outer surface can conform to irregularities around the native annulus and seal it.

In some implementations, the sealing ring 164 includes a textured outer surface, configured to promote tissue overgrowth or thrombosis, such that over time, such tissue overgrowth can improve PVL sealing against the native tissue.

In some examples, the sealing ring 164 can extend radially away from the valve frame 116 to a distance of at least 2 mm, or at least 5 mm, in a free or un-squeezed state thereof. In some examples, the sealing ring 164 can include a compressible or squeezable insert 166 and a cloth cover 165. For example, the squeezable insert 166 can be made of a silicone-based material, although other compressible materials can be used. The cloth cover 165 can be formed of any biocompatible fabric, such as, for example, polyethylene terephthalate or polyester fabric. In other implementations, the sealing ring 164 can be formed by rolling a flat sheet of cloth material to form a cylinder-like member.

While illustrated for use in a native mitral valve 70, it is to be understood that the valve assembly 100^{e} can be similarly implanted within a native tricuspid valve 80, a dilated aortic valve, or any other enlarged orifice. Utilization of the proposed valve assembly 100^{e} for implantation in such enlarged native valves or orifices can advantageously provide both adequate anchoring and PVL sealing against the native tissue, without requiring the aid of additional devices such as docking stations that can be alternatively used in such scenarios, thus simplifying the implantation procedure.

As mentioned, prosthetic valve can be expanded by balloon-inflation, by actuating mechanical actuators that apply an expansion force to the prosthetic valve, or by deploying the prosthetic valve from a sheath of the delivery apparatus so that the prosthetic valve can self-expand to its functional size. A drawback of self-expandable prosthetic valves is that they expand very rapidly when released from an outer sheath within the vasculature of a patient, which can cause trauma to the implantation site, or cause the prosthetic valve to become misaligned during implantation. In such cases, a restraining mechanism that limits the prosthetic valve's expansion can be utilized, in combination with another mechanism utilized for additional expansion. For example, a restraining mechanism can be utilized to limit the maximal expansion of the prosthetic valve, upon deployment from the sheath, to a diameter which is less than the functional size of the prosthetic valve, wherein further expansion of the prosthetic valve can be achieved by balloon inflation or utilization of mechanical expansion mechanisms to further expand the prosthetic valve to the functional size.

A self-expandable prosthetic valve can include, in some implementations, a self-expandable valve frame, configured to self-expand from a first diameter in a radially compressed state to a second diameter in a free radially expanded state. The term “free radially expanded state” refers to a final expanded state of the prosthetic valve, reaching the second diameter, when no external restricting forces are applied thereto. A valve assembly includes the self-expandable prosthetic valve, and a restriction frame disposed radially outward to, and coupled to, the self-expandable valve frame, wherein the restriction frame is configured to limit the self-expandable valve frame to a second diameter in a deployed state thereof, wherein the second diameter is less than the third diameter. A deployed state of the valve frame refers to a state in which the valve frame is not restricted by a surrounding sheath or capsule of a delivery apparatus, free to self-expand to a maximal self-expansion diameter, which can be the third diameter when a restriction frame is attached thereto, but not yet further expanded by additional expansion modalities, such as actuators or inflatable balloon, as will be further described below.

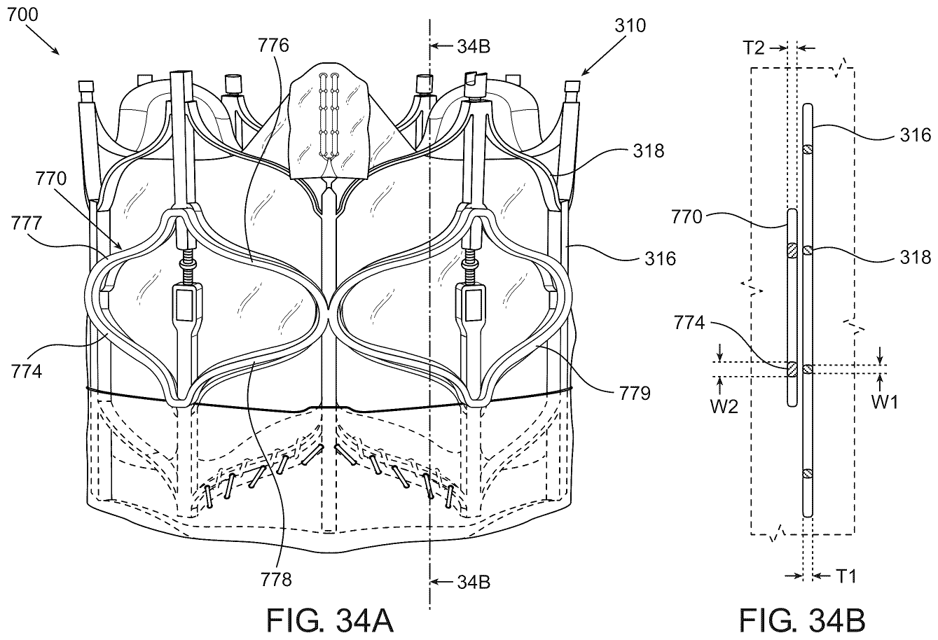

FIG. 34A shows an example of a restriction frame 770 mounted over a self-expandable prosthetic valve 310, together forming an exemplary valve assembly 700. FIG. 34B shows a cross-sectional view along line 34B-34B of FIG. 34A. The prosthetic valve 310 of valve assembly 700 can implemented according to any example described above for prosthetic valve 310 with respect to FIGS. 17A-17B, with the addition that the frame 316 is at least partially self-expandable, configured to self-expand from a first diameter in a radially compressed state to a second diameter in a free radially expanded state. That is to say, in the absence of external restricting forces, such as when the prosthetic valve 310 is deployed out of a delivery shaft 28, and not coupled to a restriction frame (770), the prosthetic valve 310 can fully expand to a second diameter, which is its maximal self-expanding diameter in such a free state, assuming that it is not bound by a narrower anatomical dimension at the site of implantation, for example.

The restriction frame 770 is disposed radially outwardly from, and is coupled to, the valve frame 316. The restriction frame 770 can be coupled to the valve frame 316 directly, such as by being sutured directly to struts 318 of the valve frame 316, or indirectly, such as by being coupled (e.g., sutured) to an outer skirt 344 that can circumscribe the valve frame 316. The restriction frame 770 comprises a plurality of restriction struts 774 configured in an annular shape. The restriction struts 774 define at least one rung, which can be referred to as a restriction first rung 776 defined by a plurality of first angled struts 777 at the proximal or outflow end of the restriction frame 770. The restriction belt 770 comprises at least one rung of struts, referred to as the restriction frame first rung 776. In some examples, the restriction frame 770 further comprises at least one additional rung of struts, distal to the restriction frame first rung 776.

As evident from the illustrated example shown in FIGS. 34A-34B, the restriction frame 770 can be generally similar to leaflet engagement frame 370 described above in conjunction with FIGS. 19-20, except that it does not necessarily include spikes. Nevertheless, it is to be understood that in other implementations, a leaflet engagement frame, such as any of the leaflet engagement frames 170^{a}, 170^{b}, 170^{c}, 170^{d}, 370^{a}, or 370^{b}, can be similarly utilized as restriction frames as well. In such cases, the leaflet engagement frame 170 or 370 is also the restriction frame, and the engagement frame struts 174 or 374 are also the restriction struts, allowing it to serve both for engaging host leaflets and for limiting the self-expandability of the valve frame.

As mentioned, the restriction frame is designed to limit the final diameter achieved during self-expandability of the self-expandable frame to a third diameter, which is less than the second diameter. This can be achieved by forming the restriction struts 774 from a more rigid material, causing the restriction frame 770 to be less expandable (in the absence of active expansion applied thereto) than the valve frame 316. Alternatively or additionally, this can be achieved by forming the restriction struts 774 to have greater dimension along at least one direction, relative to the dimensions of the frame struts 318. As shown in FIG. 34B, the frame struts 318 can have a first thickness T1 (defined in a radial direction) and a first width W1, while the restriction struts 774 can have a second thickness T2 and a second width W2. In some examples, the second width W2 is greater than the first width W1, as illustrated for example in FIG. 34B. In some examples, the second thickness T2 is greater than the first thickness T1. In some examples, the second width W2 is greater than the first width W1 and the second thickness T2 is greater than the first thickness T1.

As mentioned above, while the restriction frame 770 is illustrated in FIGS. 34A-34B to have two rungs of restriction struts, similar to the configuration shown for leaflet engagement frame 370^{a}, it is to be understood that in alternative implementations, restriction frame 770 can include any other number of strut rungs, including a single rung as shown for leaflet engagement frame 370^{b}. Moreover, while restriction frame 770 is illustrated in FIGS. 34A-34B devoid of any spikes, in other implementations, a restriction frame can include spikes, which can be angled spikes as described above for leaflet engagement frame 370 or 170, or in-plane spikes as described above for engagement frame 170^{d}.

In use, a mechanically expandable prosthetic valve 310 of a valve assembly 700 that includes a self-expandable valve frame 316 can be delivered in a radially crimped state, retained in a first diameter within a delivery shaft 28 or an outer shaft 20. Upon reaching the site of implantation, the valve assembly 700 is deployed out of the sheath of the delivery apparatus 12, such that the prosthetic valve 310 can self-expand up to the third diameter, dictated by the restriction frame 770 disposed around the valve frame 316. After reaching the third diameter, the actuators 360 can be actuated, such as via actuation assemblies 40 as described above, to further expand the valve assembly 700 to a fourth diameter, which is greater than the third diameter. The expansion force applied by the actuators 360 is designed to exceed the restrictive forces of the restriction frame 770, to allow additional expansion thereof beyond the third diameter.

While a valve assembly 700 is illustrated and described to include a prosthetic valve 310 which is both self-expandable and mechanically expandable, in other implementations, a valve assembly can include a prosthetic valve that can be self-expandable and balloon-expandable. For example, a self-expandable prosthetic valve, which can optionally resemble the prosthetic valve 110 described above, having a self-expandable valve frame 116, or any other design of a self-expandable prosthetic valve, can be provided with a restriction frame coupled to the valve frame, wherein the restriction belt can be implemented according to any example described above, including at least one rung of restriction struts, or a plurality of rungs, arranged in a similar manner to that of the four rungs of leaflet engagement frame 170^{a}, two rungs of leaflet engagement frame 170^{b}, a single rungs as shown for leaflet engagement frame 170, or any other number of rungs, and can be devoid of spikes as described for restriction frame 770 in conjunction with FIGS. 34A-34B, or include spikes that can be angled spikes, as illustrated for any of the leaflet engagement frames 170^{a}, 170^{b}, 170^{c} or in-plane spikes as described and illustrated for leaflet engagement frames 170^{d}. The restriction frame coupled to the self-expandable valve frame can be equipped with wider or thicker struts, as described above, to limit self-expandability of the valve assembly to the third diameter.

In use, such a valve assembly can be delivered in a radially crimped state, retained in a first diameter within an outer shaft 20. Upon reaching the site of implantation, the valve assembly is deployed out of the sheath of the delivery apparatus 12, such that the prosthetic valve can self-expand up to the third diameter, dictated by the restriction frame disposed around the valve frame. After reaching the third diameter, a balloon 26 disposed within the lumen of the prosthetic valve can be inflated to further expand the valve assembly to a fourth diameter, which is greater than the third diameter. The expansion force applied by the balloon 26 is designed to exceed the restrictive forces of the restriction frame, to allow additional expansion thereof beyond the third diameter.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A valve assembly, comprising:
a prosthetic valve comprising:
   a valve frame comprising a plurality of struts, wherein the valve frame is movable between a radially compressed state and a radially expanded state;
   a leaflet assembly mounted within the valve frame and comprising a plurality of leaflets configured to regulate flow through the prosthetic valve; and
   a leaflet engagement frame which is disposed radially outwardly from, and is coupled to, the valve frame, the leaflet engagement frame comprising:
   a plurality of engagement frame struts defining one or more rungs of struts, wherein the one or more rungs of struts comprises an engagement frame first rung, and wherein the engagement frame first rung further comprises a plurality of distally extending spikes that extend in a distal direction from its engagement frame struts;
wherein the plurality of distally extending spikes is configured to engage with one or more native or host leaflets positioned outside the valve assembly when the valve assembly is in a partially expanded state, and to distally fold the engaged native or host leaflets when the valve assembly is further expanded.
Example 2. The valve assembly of any example herein, particularly example 1, wherein the plurality of distally extending spikes extend at an angle α radially outward and in the distal direction from the respective struts of the engagement frame first rung.
Example 3. The valve assembly of any example herein, particularly example 1 or 2, wherein the leaflet engagement frame is devoid of any other rungs of struts other than the engagement frame first rung.
Example 4. The valve assembly of any example herein, particularly any one of examples 1 to 3, further comprising an outer skirt comprising an outer skirt outflow end and an outer skirt inflow end, wherein the outer skirt is mounted over an outer surface of the valve assembly such that the outer skirt outflow end is distal to the plurality of distally extending spikes.
Example 5. The valve assembly of any example herein, particularly example 1 or 2, wherein the one or more rungs of struts of the leaflet engagement frame comprises at least one additional rung of struts distal to the engagement frame first rung, and wherein the leaflet engagement frame further comprises a plurality of proximally extending spikes, extending in the proximal direction from the engagement frame struts of the additional rung of struts.
Example 6. The valve assembly of any example herein, particularly example 5, wherein the engagement frame struts of the additional rung of struts are devoid of distally extending spikes.
Example 7. The valve assembly of any example herein, particularly example 5 or 6, wherein the plurality of proximally extending spikes extend at an angle β radially outward and in the proximal direction from the additional rung of struts.
Example 8. The valve assembly of any example herein, particularly any one of examples 5 to 7, wherein the at least one additional rung of struts comprising the proximally extending spikes is an engagement frame second rung, and wherein the leaflet engagement frame is devoid of any other rungs of struts other than the engagement frame first rung and the engagement frame second rung.
Example 9. The valve assembly of any example herein, particularly example 8, further comprising an outer skirt comprising an outer skirt outflow end and an outer skirt inflow end, wherein the outer skirt is mounted over an outer surface of the valve assembly such that the outer skirt outflow end is distal to the distally extending spikes and the proximally
Example 10. The valve assembly of any example herein, particularly any one of examples 5 to 7, wherein the one or more rungs of struts of the leaflet engagement frame further comprises one or more intermediate rungs of struts disposed between the rungs of struts that include the distally extending spikes and the proximally extending spikes, wherein any one of the one or more intermediate rungs of struts is devoid of spikes.
Example 11. The valve assembly of any example herein, particularly example 10, further comprising an outer skirt comprising an outer skirt outflow end and an outer skirt inflow end, wherein the outer skirt is mounted over an outer surface of the valve assembly such that the outer skirt outflow end is distal to the distally extending spikes, and the outer skirt inflow end is proximal to the proximally extending spikes.
Example 12. The valve assembly of any example herein, particularly any one of examples 5 to 11, wherein each proximally extending spike terminates with a sharp distal tip.
Example 13. The valve assembly of any example herein, particularly example 7, wherein the angle β is in the range of 10-80 degrees.
Example 14. The valve assembly of any example herein, particularly example 13, wherein the angle β is in the range of 20-70 degrees.
Example 15. The valve assembly of any example herein, particularly example 14, wherein the angle β is in the range of 30-60 degrees.
Example 16. The valve assembly of any example herein, particularly any one of examples 1 to 15, wherein each distally extending spike terminates with a sharp distal tip.
Example 17. The valve assembly of any example herein, particularly example 2, wherein the angle α is in the range of 10-80 degrees.
Example 18. The valve assembly of any example herein, particularly example 17, wherein the angle α is in the range of 20-70 degrees.
Example 19. The valve assembly of any example herein, particularly example 18, wherein the angle α is in the range of 30-60 degrees.
Example 20. The valve assembly of any example herein, particularly any one of examples 1 to 19, wherein the plurality of struts of the valve frame define a plurality of rungs of struts, comprising:
a valve frame outflow rung;
a valve frame inflow rung; and
at least one valve frame intermediate rung disposed between the valve frame outflow rung and the valve frame inflow rung,
wherein the struts of the engagement frame first rung are aligned with struts of a valve frame intermediate rung which is distal to the valve frame outflow rung.
Example 21. The valve assembly of any example herein, particularly example 20, wherein the at least one valve frame intermediate rung comprises: a valve frame first intermediate rung, a valve frame second intermediate rungs, and a valve frame third intermediate rungs, wherein the struts of the valve frame further comprise valve frame distal axial struts interconnecting struts of the valve frame inflow rung with struts of the valve frame third intermediate rung, and wherein the struts of the engagement frame first rung are aligned with the struts of the valve frame first intermediate rung.
Example 22. The valve assembly of any example herein, particularly example 20, wherein the at least one valve frame intermediate rung comprises a valve frame first intermediate rung and a valve frame second intermediate rung, wherein the struts of the engagement frame first rung are aligned with the struts of the valve frame first intermediate rung.
Example 23. The valve assembly of any example herein, particularly example 22, wherein the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.
Example 24. A leaflet engagement frame for a valve assembly, the leaflet engagement frame comprising:
a plurality of interconnected engagement frame struts defining one or more rungs of struts, wherein the one or more rungs of struts comprises an engagement frame first rung; and
a plurality of distally extending spikes, extending in the distal direction solely from the struts of the engagement frame first rung,
wherein the plurality of distally extending spikes are configured to engage with one or more native or host leaflets positioned outside the leaflet engagement frame when the leaflet engagement frame is in a partially expanded state, and to distally fold the engaged native or host leaflets when the leaflet engagement frame is further expanded.
Example 25. The leaflet engagement frame of any example herein, particularly example 24, wherein the distally extending spikes are extending at a sharp angle α radially outward and in the distal direction from the respective struts of the engagement frame first rung.
Example 26. The leaflet engagement frame of any example herein, particularly example 24 or 25, devoid of any other rungs of struts other than the engagement frame first rung.
Example 27. The leaflet engagement frame of any example herein, particularly example 24 or 25, wherein the one or more rungs of struts comprises at least one additional rung of struts distal to the engagement frame first rung, and wherein the leaflet engagement frame further comprises a plurality of proximally extending spikes, extending in the proximal direction from the engagement frame struts of the additional rung of struts.
Example 28. The leaflet engagement frame of any example herein, particularly example 27, wherein the proximally extending spikes are extending at a sharp angle β radially outward and in the proximal direction from the additional rung of struts.
Example 29. The leaflet engagement frame of any example herein, particularly example 27 or 28, wherein the at least one additional rung of struts which comprises the proximally extending spikes, is an engagement frame second rung, and wherein the leaflet engagement frame is devoid of any other rungs of struts other than the engagement frame first rung and the engagement frame second rung.
Example 30. The leaflet engagement frame of any example herein, particularly example 27 or 28, further comprising one or more intermediate rungs of struts disposed between the rungs of struts that include the distally extending spikes and the proximally extending spikes, wherein any of the one or more intermediate rungs of struts is devoid of spikes.
Example 31. The leaflet engagement frame of any example herein, particularly any one of examples 24 to 30, wherein each proximally extending angled spike terminates with a sharp distal tip.
Example 32. The leaflet engagement frame of any example herein, particularly example 28, wherein the angle β is in the range of 10-80 degrees.
Example 33. The leaflet engagement frame of any example herein, particularly example 32, wherein the angle β is in the range of 20-70 degrees.
Example 34. The leaflet engagement frame of any example herein, particularly example 33, wherein the angle β is in the range of 30-60 degrees.
Example 35. The leaflet engagement frame of any example herein, particularly any one of examples 24 to 34, wherein each distally extending angled spike terminates with a sharp distal tip.
Example 36. The leaflet engagement frame of any example herein, particularly example 25, wherein the angle α is in the range of 10-80 degrees.
Example 37. The leaflet engagement frame of any example herein, particularly example 36, wherein the angle α is in the range of 20-70 degrees.
Example 38. The leaflet engagement frame of any example herein, particularly example 37, wherein the angle α is in the range of 30-60 degrees.
Example 39. A valve assembly, comprising:
a prosthetic valve comprising:
   a valve frame movable between a radially compressed state and a radially expanded state, the valve frame comprising a plurality of frame rungs, each frame rung comprising interconnected struts, the plurality of frame rungs comprising:
      a valve frame outflow rung, which is proximal to any other rung of the plurality of frame rungs;
      a valve frame inflow rung, which is distal to any other rung of the plurality of frame rungs; and
      at least one valve frame intermediate rung disposed between the valve frame outflow rung and the valve frame inflow rung, the at least one valve frame intermediate rung comprising a valve frame first intermediate rung,
   a leaflet assembly mounted within the valve frame and comprising a plurality of leaflets configured to regulate flow through the prosthetic valve; and
   at least one proximal leaflet engagement wire wrapped around struts of at least one valve frame intermediate rung,
wherein the at least one proximal leaflet engagement wire is configured to engage one or more native or host leaflets positioned outside the valve assembly when the valve assembly is in a partially expanded state, and to distally fold the engaged native or host leaflets when the valve assembly is further expanded.
Example 40. The valve assembly of any example herein, particularly example 39, wherein the at least one valve frame intermediate rung around which the at least one proximal leaflet engagement wire is wrapped, comprises a single valve frame intermediate rung.
Example 41. The valve assembly of any example herein, particularly example 38 or 40, wherein all other frame rungs are devoid of engagement wires wrapped there-over.
Example 42. The valve assembly of any example herein, particularly any one of examples 39 to 41, further comprising an outer skirt comprising an outer skirt outflow end and an outer skirt inflow end, wherein the outer skirt is mounted over an outer surface of the valve assembly such that the outer skirt outflow end is distal to the proximal leaflet engagement wire.
Example 43. The valve assembly of any example herein, particularly example 40, further comprising at least one distal leaflet engagement wire wrapped around struts of at least one other rung of the plurality of frame rungs, wherein the at least one other rung is distal to the proximal leaflet engagement wire.
Example 44. The valve assembly of any example herein, particularly example 43, wherein the at least one other rung comprises a single rung of the plurality of frame rungs.
Example 45. The valve assembly of any example herein, particularly example 43 or 44, wherein all other valve frame rungs, except the single rung around which the proximal leaflet engagement wire is wrapped and the single rung around which the distal leaflet engagement wire is wrapped, are devoid of engagement wires wrapped there-over.
Example 46. The valve assembly of any example herein, particularly any one of examples 43 to 45, wherein the at least one other rung around which the distal leaflet engagement wire is wrapped comprises the valve frame inflow rung.
Example 47. The valve assembly of any example herein, particularly example 46, further comprising an outer skirt comprising an outer skirt outflow end and an outer skirt inflow end, wherein the outer skirt is mounted over an outer surface of the valve assembly such that the outer skirt outflow end is distal to the proximal leaflet engagement wire, and the outer skirt inflow end is proximal to the distal leaflet engagement wire.
Example 48. The valve assembly of any example herein, particularly any one of examples 43 to 45, wherein the at least one valve frame intermediate rung further comprises a valve frame second intermediate rung, and wherein the at least one other rung around which the distal leaflet engagement wire is wrapped comprises the valve frame second intermediate rung.
Example 49. The valve assembly of any example herein, particularly example 48, further comprising an outer skirt comprising an outer skirt outflow end and an outer skirt inflow end, wherein the outer skirt is mounted over an outer surface of the valve assembly such that the outer skirt outflow end is distal to the proximal leaflet engagement wire and the distal leaflet engagement wire.
Example 50. The valve assembly of any example herein, particularly example 48, wherein the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.
Example 51. A prosthetic valve, comprising:
a valve frame movable between a radially compressed state and a radially expanded state, the valve frame comprising a plurality of rungs of interconnected struts, comprising:
   a valve frame outflow rung;
   a valve frame inflow rung; and
   at least one valve frame intermediate rung disposed between the valve frame outflow rung and the valve frame inflow rung, the at least one valve frame intermediate rung comprising a valve frame first intermediate rung,
   a plurality of distally extending spikes, integrally formed with the struts of a single valve frame intermediate rung, and extending in the distal direction therefrom; and
   a leaflet assembly mounted within the valve frame and comprising a plurality of leaflets configured to regulate flow through the prosthetic valve,
wherein the plurality of distally extending spikes are configured to engage with one or more native or host leaflets positioned outside the prosthetic valve when the prosthetic valve is in a partially expanded state, and to distally fold the engaged native or host leaflets when the prosthetic valve is further expanded.
Example 52. The prosthetic valve of any example herein, particularly example 51, wherein the distally extending spikes are extending at a sharp angle α radially outward and in the distal direction from the respective struts of the valve frame first intermediate rung.
Example 53. The prosthetic valve of any example herein, particularly example 51 or 52, further comprising an outer skirt comprising an outer skirt outflow end and an outer skirt inflow end, wherein the outer skirt is mounted over an outer surface of the prosthetic valve such that the outer skirt outflow end is distal to the distally extending spikes.
Example 54. The prosthetic valve of any example herein, particularly example 51 or 52, further comprising a plurality of proximally extending spikes integrally formed with an additional single valve frame rung which is distal to the distally extending spike, and extending in the proximal direction therefrom.
Example 55. The prosthetic valve of any example herein, particularly example 54, wherein the proximally extending spikes are extending at a sharp angle β radially outward and in the proximal direction from the additional valve frame rung.
Example 56. The prosthetic valve of any example herein, particularly example 54 or 55, wherein all other valve frame rungs, except the single rung comprising the distally extending spikes and the single rung comprising the proximally extending spikes, are devoid of integrally formed spikes.
Example 57. The prosthetic valve of any example herein, particularly example 54 or 55, wherein the proximally extending spikes are integrally formed with struts of the valve frame inflow rung.
Example 58. The prosthetic valve of any example herein, particularly example 57, further comprising an outer skirt comprising an outer skirt outflow end and an outer skirt inflow end, wherein the outer skirt is mounted over an outer surface of the valve assembly such that the outer skirt outflow end is distal to the distally extending spikes, and the outer skirt inflow end is proximal to the proximally extending spikes.
Example 59. The prosthetic valve of any example herein, particularly example 54 or 55, wherein the at least one valve frame intermediate rung further comprises a valve frame second intermediate rung, and wherein the proximally extending spikes are integrally formed with struts of the valve frame second intermediate rung.
Example 60. The prosthetic valve of any example herein, particularly example 59, further comprising an outer skirt comprising an outer skirt outflow end and an outer skirt inflow end, wherein the outer skirt is mounted over an outer surface of the prosthetic valve such that the outer skirt outflow end is distal to the distally extending spikes and the proximally extending spikes.
Example 61. The prosthetic valve of any example herein, particularly example 60, wherein the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.
Example 62. The prosthetic valve of any example herein, particularly any one of examples 54 to 61, wherein each proximally extending spike terminates with a sharp distal tip.
Example 63. The prosthetic valve of any example herein, particularly example 60, wherein the angle β is in the range of 10-80 degrees.
Example 64. The prosthetic valve of any example herein, particularly example 60, wherein the angle β is in the range of 20-70 degrees.
Example 65. The prosthetic valve of any example herein, particularly example 60, wherein the angle β is in the range of 30-60 degrees.
Example 66. The prosthetic valve of any example herein, particularly any one of examples 54 to 61, wherein each distally extending angled spikes terminates with a sharp distal tip.
Example 67. The prosthetic valve of any example herein, particularly example 60, wherein the angle α is in the range of 10-80 degrees.
Example 68. The prosthetic valve of any example herein, particularly example 60, wherein the angle α is in the range of 20-70 degrees.
Example 69. The prosthetic valve of any example herein, particularly example 60, wherein the angle α is in the range of 30-60 degrees.
Example 70. A method comprising:
positioning a valve assembly between native or host leaflets within a patient's body, the valve assembly comprising:
   a prosthetic valve comprising a valve frame movable between a radially compressed state and a radially expanded state, and a leaflet assembly mounted within the valve frame and comprising a plurality of leaflets configured to regulate flow through the prosthetic valve; and
   a leaflet engagement frame which is disposed radially outwardly from, and is coupled to, the valve frame, the leaflet engagement frame comprising a plurality of distally extending angled spikes, extending at a sharp angle α radially outward and in the distal direction solely from struts of an engagement frame first rung of the leaflet engagement frame,
partially expanding the valve assembly at least until the distally extending angled spikes engage with the native or host leaflets; and
further expanding the valve assembly such that the distally extending angled spikes remain engaged with the native or host leaflets, and drag the native or host leaflets distally to a folded configuration of the native or host leaflets.
Example 71. The method of any example herein, particularly example 70, wherein the leaflet engagement frame is devoid of any other rungs of struts other than the engagement frame first rung.
Example 72. The method of any example herein, particularly example 71, further comprising a step of advancing the valve assembly in a distal direction after the step of partially expanding the valve assembly, such that the distally extending angled spikes remain engaged with the native or host leaflets and drag the native or host leaflets along with the valve assembly during its advancement in the partially expanded state.
Example 73. The method of any example herein, particularly example 70, wherein the leaflet engagement frame further comprises a plurality of proximally extending angled spikes, extending at a sharp angle β radially outward and in the proximal direction solely from struts of an additional rung of the leaflet engagement frame, distal to the distally extending angled spikes.
Example 74. The method of any example herein, particularly example 73, wherein the step of partially expanding the valve assembly comprises partially expanding the valve assembly at least until the proximally extending angled spikes engage with a native annulus or with the native or host leaflets.
Example 75. The method of any example herein, particularly example 73 or 74, wherein the leaflet engagement frame comprises additional rungs of struts between the distally extending angled spikes and the proximally extending angled spikes, and wherein the additional rungs of struts are devoid of angled spikes.
Example 76. The method of any example herein, particularly any one of examples 73 to 75, wherein each proximally extending angled spike terminates with a sharp distal tip.
Example 77. The method of any example herein, particularly any one of examples 73 to 76, wherein the angle β is in the range of 10-80 degrees.
Example 78. The method of any example herein, particularly example 77, wherein the angle β is in the range of 20-70 degrees.
Example 79. The method of any example herein, particularly example 78, wherein the angle β is in the range of 30-60 degrees.
Example 80. The method of any example herein, particularly any one of examples 70 to 79, wherein each distally extending angled spike terminates with a sharp distal tip.
Example 81. The method of any example herein, particularly any one of examples 70 to 80, wherein the angle α is in the range of 10-80 degrees.
Example 82. The method of any example herein, particularly example 81, wherein the angle α is in the range of 20-70 degrees.
Example 83. The method of any example herein, particularly example 82, wherein the angle α is in the range of 30-60 degrees.
Example 84. A method comprising:
positioning a valve assembly between native or host leaflets within a patient's body, the valve assembly comprising:
   a prosthetic valve comprising:
      a valve frame movable between a radially compressed state and a radially expanded state, the frame comprising: a valve frame outflow rung, a valve frame inflow rung, and at least one valve frame intermediate rung disposed between the valve frame outflow rung and the valve frame inflow rung; and
      a leaflet assembly mounted within the valve frame and comprising a plurality of leaflets configured to regulate flow through the prosthetic valve; and
   at least one proximal leaflet engagement wire wrapped around struts of a single valve frame intermediate rung;
partially expanding the valve assembly at least until the proximal leaflet engagement wire engages with the native or host leaflets; and
further expanding the valve assembly such that the proximal leaflet engagement wire remains engaged with the native or host leaflets, and drags the native or host leaflets distally to a folded configuration of the native or host leaflets.
Example 85. The method of any example herein, particularly example 84, wherein the at least one proximal leaflet engagement wire comprises a single continuous proximal leaflet engagement wire, wrapped around all struts of the valve frame intermediate rung.
Example 86. The method of any example herein, particularly example 84 or 85, wherein all other rungs of struts of the valve frame are devoid of wires wrapped there-over.
Example 87. The method of any example herein, particularly example 86, further comprising a step of advancing the valve assembly in a distal direction after the step of partially expanding the valve assembly, such that the proximal leaflet engagement wire remains engaged with the native or host leaflets and drags the leaflets along with the valve assembly during its advancement in the partially expanded state.
Example 88. The method of any example herein, particularly example 87, wherein the leaflet engagement frame further comprises at least one distal leaflet engagement wire wrapped around struts of another single valve frame rung which is distal to the proximal leaflet engagement wire.
Example 89. The method of any example herein, particularly example 88, wherein the at least one distal leaflet engagement wire comprises a single distal leaflet engagement wire, wrapped around all struts of the valve frame rung.
Example 90. The method of any example herein, particularly example 88 or 89, wherein the step of partially expanding the valve assembly comprises partially expanding the valve assembly at least until the distal leaflet engagement wire engages with a native annulus or with the native or host leaflets.
Example 91. The method of any example herein, particularly any one of examples 88 to 90, wherein all other valve frame rungs, except the single rung around which the proximal leaflet engagement wire is wrapped and the single rung around which the distal leaflet engagement wire is wrapped, are devoid of wires wrapped there-over.
Example 92. The method of any example herein, particularly any one of examples 88 to 91, wherein the distal leaflet engagement wire is wrapped around struts of the valve frame inflow rung.
Example 93. The method of any example herein, particularly any one of examples 88 to 91, wherein the at least one valve frame intermediate rung further comprises a valve frame second intermediate rung, and wherein the distal leaflet engagement wire is wrapped around struts of the valve frame second intermediate rung.
Example 94. A method comprising:
positioning a prosthetic valve between native or host leaflets within a patient's body, the prosthetic valve comprising:
   a valve frame movable between a radially compressed state and a radially expanded state, the valve frame comprising: a valve frame outflow rung, a valve frame inflow rung, and at least one valve frame intermediate rung disposed between the valve frame outflow rung and the valve frame inflow rung;
   a plurality of distally extending angled spikes, integrally formed with struts of a single valve frame intermediate rung, and extending at a sharp angle α radially outward and in the distal direction therefrom; and
   a leaflet assembly mounted within the valve frame and comprising a plurality of leaflets configured to regulate flow through the prosthetic valve; and
partially expanding the prosthetic valve at least until the distally extending angled spikes engage with the native or host leaflets; and
further expanding the prosthetic valve such that the distally extending angled spikes remain engaged with the native or host leaflets, and drag the native or host leaflets distally to a folded configuration of the native or host leaflets.
Example 95. The method of any example herein, particularly example 94, wherein all other rungs of the valve frame are devoid of angled spikes.
Example 96. The method of any example herein, particularly example 95, further comprising a step of advancing the prosthetic valve in a distal direction after the step of partially expanding the prosthetic valve, such that the distally extending angled spikes remain engaged with the native or host leaflets and drag the native or host leaflets along with the prosthetic valve during its advancement in the partially expanded state.
Example 97. The method of any example herein, particularly example 94, wherein the prosthetic valve further comprises a plurality of proximally extending angled spikes integrally formed with another single valve frame rung which is distal to the distally extending angled spike, and extending at a sharp angle β radially outward and in the proximal direction therefrom.
Example 98. The method of any example herein, particularly example 97, wherein the step of partially expanding the prosthetic valve comprises partially expanding the valve assembly at least until the proximally extending angled spikes engage with a native annulus or with the native or host leaflets.
Example 99. The method of any example herein, particularly example 97 or 98, wherein all other valve frame rungs, except the single rung comprising the distally extending angled spikes and the single rung comprising the proximally extending angled spikes, are devoid of integrally formed spikes.
Example 100. The method of any example herein, particularly any one of examples 97 to 99, wherein the proximally extending angled spikes are integrally formed with struts of the valve frame inflow rung.
Example 101. The method of any example herein, particularly any one of examples 97 to 99, wherein the at least one valve frame intermediate rung further comprise a valve frame second intermediate rung, and wherein the proximally extending angled spikes are integrally formed with struts of the valve frame second intermediate rung.
Example 102. The method of any example herein, particularly any one of examples 97 to 101, wherein each proximally extending angled spike terminates with a sharp distal tip.
Example 103. The method of any example herein, particularly any one of examples 97 to 102, wherein the angle β is in the range of 10-80 degrees.
Example 104. The method of any example herein, particularly example 103, wherein the angle β is in the range of 20-70 degrees.
Example 105. The method of any example herein, particularly example 104, wherein the angle β is in the range of 30-60 degrees.
Example 106. The method of any example herein, particularly any one of examples 94 to 105, wherein each distally extending angled spike terminates with a sharp distal tip.
Example 107. The method of any example herein, particularly any one of examples 94 to 106, wherein the angle α is in the range of 10-80 degrees.
Example 108. The method of any example herein, particularly example 107, wherein the angle α is in the range of 20-70 degrees.
Example 109. The method of any example herein, particularly example 108, wherein the angle α is in the range of 30-60 degrees.
Example 110. A method comprising:
positioning a valve assembly between native or host leaflets within a patient's body, the valve assembly comprising:
   a prosthetic valve comprising a valve frame movable between a radially compressed state and a radially expanded state, and a leaflet assembly mounted within the valve frame and comprising a plurality of leaflets configured to regulate flow through the prosthetic valve; and
   a leaflet engagement frame which is disposed radially outwardly from, and is coupled to, the valve frame, the leaflet engagement frame comprising a plurality of distally extending angled spikes, extending at a sharp angle α radially outward and in the distal direction solely from struts of an engagement frame first rung of the leaflet engagement frame;
partially expanding the valve assembly;
confirming that the valve assembly is in proper position in the patient's body using tactile feedback from the valve assembly created by contact of the distally extending angled spikes with the native or host leaflets; and
further expanding the valve assembly in a desired position within the patient's body.
Example 111. The method of any example herein, particularly example 110, wherein the leaflet engagement frame is devoid of any other rungs of struts other than the engagement frame first rung.
Example 112. The method of any example herein, particularly example 110 or 111, wherein each distally extending angled spike terminates with a sharp distal tip.
Example 113. The method of any example herein, particularly any one of examples 110 to 112, wherein the angle α is in the range of 10-80 degrees.
Example 114. The method of any example herein, particularly example 113, wherein the angle α is in the range of 20-70 degrees.
Example 115. The method of any example herein, particularly example 114, wherein the angle α is in the range of 30-60 degrees.
Example 116. A valve assembly comprising:
a prosthetic valve comprising:
   a valve frame defined between a valve outflow end and a valve inflow end, wherein the valve frame movable between a radially compressed state and a radially expanded state, and wherein the valve frame comprises intersecting struts that define at least one row of circumferentially extending frame cells; and
   a leaflet assembly mounted within the valve frame and comprising a plurality of leaflets configured to regulate flow through the prosthetic valve; and
a leaflet engagement frame which is disposed radially outwardly from, and is coupled to, the valve frame, the leaflet engagement frame comprising:
   first struts defined along an engagement frame first rung;
   second struts defined along an engagement frame second rung, distal to the engagement frame first rung;
   three engagement cells, each engagement cell defined by two first struts extending from an engagement frame outflow apex to engagement frame intermediate junctions, and two second struts extending from the two engagement frame intermediate junctions to an engagement frame inflow apex; and
   three distally extending hooks, wherein each distally extending hook comprises a curved portion connected on one end to a corresponding engagement frame outflow apex, and an opposite free end positioned radially away from the corresponding outflow apex and oriented in a distal direction,
wherein the distally extending hooks are configured to capture, in the curved portions, proximal ends of one or more native or host leaflets, positioned outside the valve assembly, when the valve assembly is in a partially expanded state, and to distally fold the captured native or host leaflets when the valve assembly is further expanded.
Example 117. The valve assembly of any example herein, particularly example 116, wherein the valve frame comprises a row with more than three cells.
Example 118. The valve assembly of any example herein, particularly example 117, wherein the valve frame comprises a row with six cells.
Example 119. The valve assembly of any example herein, particularly example 118, wherein the valve frame comprises a row with twelve cells.
Example 120. The valve assembly of any example herein, particularly any one of examples 116 to 119, wherein the engagement frame outflow apices are distal to the valve outflow end.
Example 121. The valve assembly of any example herein, particularly any one of examples 116 to 120, wherein the first struts and the second struts of the leaflet engagement frame are not aligned with any of the struts of the valve frame.
Example 122. The valve assembly of any example herein, particularly any one of examples 116 to 121, wherein the second struts are longer than the first struts.
Example 123. The valve assembly of any example herein, particularly any one of examples 116 to 122, further comprising an outer skirt comprising an outer skirt outflow end and an outer skirt inflow end, wherein the outer skirt is mounted over an outer surface of the valve assembly such that the outer skirt outflow end is distal to the distally extending hooks.
Example 124. The valve assembly of any example herein, particularly any one of examples 116 to 123, further comprising three proximally extending angled spikes, each extending at a sharp angle β radially outward and in the proximal direction from a corresponding engagement frame inflow apex.
Example 125. The valve assembly of any example herein, particularly example 124, wherein each proximally extending angled spike terminates with a sharp distal tip.
Example 126. The valve assembly of any example herein, particularly example 123 or 125, wherein the angle β is in the range of 10-80 degrees.
Example 127. The valve assembly of any example herein, particularly example 126, wherein the angle β is in the range of 20-70 degrees.
Example 128. The valve assembly of any example herein, particularly example 127, wherein the angle β is in the range of 30-60 degrees.
Example 129. A method comprising:
positioning a valve assembly between native or host leaflets within a patient's body, the valve assembly comprising:
   a prosthetic valve comprising:
      a valve frame movable between a radially compressed state and a radially expanded state, the valve frame defined between a valve inflow end and a valve outflow end and comprises intersecting struts that define at least one row of circumferentially extending frame cells; and
      a leaflet assembly mounted within the valve frame and comprising a plurality of leaflets configured to regulate flow through the prosthetic valve; and
   a leaflet engagement frame which is disposed radially outwardly from, and is coupled to, the valve frame, the leaflet engagement frame comprising:
      three engagement cells, each engagement cell defined by two first struts extending from an engagement frame outflow apex to engagement frame intermediate junctions, and two second struts extending from the two engagement frame intermediate junctions to an engagement frame inflow apex; and
      three distally extending hooks, each distally extending hook comprising a curved portion connected on one end to a corresponding engagement frame outflow apex, and an opposite free end positioned radially away from the corresponding outflow apex and oriented in a distal direction,
partially expanding the valve assembly at least until the distally extending hooks capture proximal ends of native or host leaflets within the curved portions; and
further expanding the valve assembly such that the distally extending hooks push against the native or host leaflets in a distal direction to a folded configuration of the native or host leaflets.
Example 130. The method of any example herein, particularly example 129, wherein the valve frame comprises a row with more than three cells.
Example 131. The method of any example herein, particularly example 130, wherein the valve frame comprises a row with six cells.
Example 132. The method of any example herein, particularly example 131, wherein the valve frame comprises a row with twelve cells.
Example 133. The method of any example herein, particularly any one of examples 129 to 132, wherein the engagement frame outflow apices are distal to the valve outflow end.
Example 134. The method of any example herein, particularly any one of examples 129 to 133, wherein the first struts and the second struts of the leaflet engagement frame are not aligned with any of the struts of the valve frame.
Example 135. The method of any example herein, particularly any one of examples 129 to 134, wherein the second struts are longer than the first struts.
Example 136. The method of any example herein, particularly any one of examples 129 to 135, further comprising a step of advancing the valve assembly in a distal direction after the step of partially expanding the valve assembly, such that the distally extending hooks push against the native or host leaflets, so as to displace their proximal ends in a distal direction along with the valve assembly during its advancement in the partially expanded state.
Example 137. The method of any example herein, particularly any one of examples 129 to 135, wherein the leaflet engagement frame further comprises three proximally extending angled spikes, each extending at a sharp angle β radially outward and in the proximal direction from a corresponding engagement frame inflow apex.
Example 138. The method of any example herein, particularly example 137, wherein each proximally extending angled spike terminates with a sharp distal tip.
Example 139. The method of any example herein, particularly example 137 or 138, wherein the angle β is in the range of 10-80 degrees.
Example 140. The method of any example herein, particularly example 139, wherein the angle β is in the range of 20-70 degrees.
Example 141. The method of any example herein, particularly example 140, wherein the angle β is in the range of 30-60 degrees.
Example 142. A valve assembly, comprising:
a prosthetic valve comprising:
   a valve frame movable between a radially compressed state and a radially expanded state;
   a leaflet assembly mounted within the valve frame and comprising a plurality of leaflets configured to regulate flow through the prosthetic valve; and
   an outer skirt attached to the valve frame and comprising a floating fiber portion disposed between an outer skirt outflow end and an outer skirt inflow end, the floating fiber portion comprising a plurality of filaments; and
a leaflet engagement frame which is disposed around, and is coupled to, the outer skirt, the leaflet engagement frame comprising at least one rung of engagement frame struts, and a plurality of spikes extending from the engagement frame struts,
wherein the spikes are configured to engage with a native tissue outside the valve assembly to irritate the native tissue.
Example 143. The valve assembly of any example herein, particularly example 142, wherein the outer skirt further comprises an outflow woven portion and an inflow woven portion, and wherein the floating fiber portion is bounded between the outflow woven portion and the inflow woven portion.
Example 144. The valve assembly of any example herein, particularly example 143, wherein the leaflet engagement frame is disposed between the outflow woven portion and the inflow woven portion.
Example 145. The valve assembly of any example herein, particularly any one of examples 142 to 144, wherein at least some of the plurality of filaments are textured filaments.
Example 146. The valve assembly of any example herein, particularly any one of examples 142 to 145, wherein the plurality of filaments curves radially outward in the radially expanded state.
Example 147. The valve assembly of any example herein, particularly any one of examples 142 to 146, wherein the plurality of filaments comprises fibers, the fibers having a diameter of from 1 μm to 20 μm to promote thrombus formation around the outer skirt
Example 148. The valve assembly of any example herein, particularly any one of examples 142 to 147, wherein the at least one rung of engagement frame struts comprises two rungs of engagement frame struts.
Example 149. The valve assembly of any example herein, particularly any one of examples 142 to 148, wherein the spikes extend along a circumferential plane defined by the engagement frame struts.
Example 150. The valve assembly of any example herein, particularly any one of examples 142 to 148, wherein the spikes extend at an angle radially outward and in an axial direction from the engagement frame struts.
Example 151. A valve assembly, comprising:
a prosthetic valve comprising:
   a valve frame movable between a radially compressed state and a radially expanded state;
   a leaflet assembly mounted within the valve frame and comprising a plurality of leaflets configured to regulate flow through the prosthetic valve; and
   an outer skirt attached to the valve frame and comprising a sealing ring extending radially away from the valve frame; and
a leaflet engagement frame which is disposed radially outwardly from, and is coupled to, the valve frame, the leaflet engagement frame comprising at least one rung of engagement frame struts, and a plurality of spikes extending from the engagement frame struts;
wherein the spikes are configured to contact native tissue to help secure the valve assembly at an implantation location.
Example 152. The valve assembly of any example herein, particularly example 151, wherein the leaflet engagement frame is directly sutured to the valve frame.
Example 153. The valve assembly of any example herein, particularly example 151, wherein the outer skirt further comprises a base layer disposed around and coupled to the valve frame, wherein the sealing ring is coupled to the base layer, and wherein the leaflet engagement frame is coupled to the base layer.
Example 154. The valve assembly of any example herein, particularly any one of examples 151 to 153, wherein the sealing ring is impervious to blood flow therethrough.
Example 155. The valve assembly of any example herein, particularly any one of examples 151 to 154, wherein the sealing ring comprises a textured outer surface configured to encourage tissue overgrowth.
Example 156. The valve assembly of any example herein, particularly any one of examples 151 to 155, wherein the sealing ring is compressible.
Example 157. The valve assembly of any example herein, particularly example 156, wherein the sealing ring comprises a compressible insert and a cloth cover.
Example 158. The valve assembly of any example herein, particularly example 157, wherein the compressible insert comprises a silicone-based material.
Example 159. The valve assembly of any example herein, particularly any one of examples 151 to 157, wherein the sealing ring comprises a flat sheet of cloth rolled over itself to form a cylinder-like form.
Example 160. The valve assembly of any example herein, particularly any one of examples 151 to 159, wherein the sealing ring is disposed around an inflow end of the prosthetic valve.
Example 161. The valve assembly of any example herein, particularly any one of examples 151 to 160, wherein the sealing ring extends radially away from the valve frame to a distance of at least 2 mm
Example 162. The valve assembly of any example herein, particularly any one of examples 151 to 160, wherein the sealing ring extends radially away from the valve frame to a distance of at least 5 mm
Example 163. The valve assembly of any example herein, particularly any one of examples 151 to 162, wherein the at least one rung of engagement frame struts comprises two rungs of engagement frame struts.
Example 164. The valve assembly of any example herein, particularly any one of examples 151 to 163, wherein the spikes extend along a circumferential plane defined by the engagement frame struts.
Example 165. The valve assembly of any example herein, particularly any one of examples 151 to 163, wherein the spikes extend at a sharp angle radially outward and in an axial direction from the engagement frame struts.
Example 166. A valve assembly, comprising:
a prosthetic valve comprising:
   a valve frame configured to self-expand from a first diameter in a radially compressed state to a third diameter in a free radially expanded state;
   a leaflet assembly mounted within the valve frame and comprising a plurality of leaflets configured to regulate flow through the prosthetic valve; and
   a restriction frame which is disposed radially outwardly from, and is coupled to, the valve frame, the restriction frame comprising at least one rung of intersecting restriction struts;
wherein the restriction frame is configured to limit the valve frame to a second diameter in a deployed state thereof, wherein the second diameter is less than the third diameter.
Example 167. The valve assembly of any example herein, particularly example 166, wherein the valve frame comprises a plurality of intersecting struts, the struts of the valve frame defining a first thickness and a first width, and wherein each the restriction struts define a second thickness and a second width.
Example 168. The valve assembly of any example herein, particularly example 167, wherein the second width is greater than the first width.
Example 169. The valve assembly of any example herein, particularly example 167 or 168, wherein the second thickness is greater than the first thickness.
Example 170. The valve assembly of any example herein, particularly any one of examples 166 to 169, wherein the restriction frame is directly sutured to the valve frame.
Example 171. The valve assembly of any example herein, particularly any one of examples 166 to 169, wherein the prosthetic valve further comprises an outer skirt disposed around and coupled to the valve frame, and wherein the restriction frame is coupled to the outer skirt.
Example 172. The valve assembly of any example herein, particularly any one of examples 166 to 171, wherein the at least one rung of engagement frame struts comprises a single rung of zig-zagged engagement frame struts.
Example 173. The valve assembly of any example herein, particularly any one of examples 166 to 171, wherein the at least one rung of engagement frame struts comprises two rungs of engagement frame struts.
Example 174. The valve assembly of any example herein, particularly any one of examples 166 to 173, wherein the restriction frame further comprises a plurality of spikes extending from the engagement frame struts.
Example 175. The valve assembly of any example herein, particularly example 174, wherein the spikes extend along a circumferential plane defined by the engagement frame struts.
Example 176. The valve assembly of any example herein, particularly example 174, wherein the spikes extend at a sharp angle radially outward and in an axial direction from the engagement frame struts.
Example 177. The valve assembly of any example herein, particularly any one of examples 166 to 176, wherein the prosthetic valve further comprises a plurality of actuators configured to produce radial expansion of the valve frame to a diameter greater than the third diameter.
Example 178. The valve assembly of any example herein, particularly example 177, wherein each actuator comprises a threaded rod engaged with a corresponding threaded nut.
Example 179. The valve assembly of any example herein, particularly example 178, wherein each threaded rod is configured to rotate such that inflow apices and outflow apices of the valve frame move axially toward one another and radially expand the valve frame.
Example 180. A valve assembly, comprising:
a prosthetic valve comprising:
   a valve frame comprising a plurality of struts, wherein the valve frame is movable between a radially compressed state and a radially expanded state;
   a leaflet assembly mounted within the valve frame and comprising a plurality of leaflets configured to regulate flow through the prosthetic valve; and
   a leaflet engagement frame which is disposed radially outwardly from, and is coupled to, the valve frame, the leaflet engagement frame comprising:
      a plurality of engagement frame struts defining one or more rungs of struts, wherein the one or more rungs of struts comprises an engagement frame first rung; and
      a plurality of distally extending spikes, extending in the distal direction solely from the struts of the engagement frame first rung,
wherein the plurality of distally extending spikes are configured to engage with one or more native or host leaflets positioned outside the valve assembly when the valve assembly is in a partially expanded state, and to distally fold the engaged native or host leaflets when the valve assembly is further expanded.
Example 181. The valve assembly of any example herein, particularly example 180, wherein the distally extending spikes are extending at a sharp angle α radially outward and in the distal direction from the respective struts of the engagement frame first rung.
Example 182. The valve assembly of any example herein, particularly example 180 or 181, wherein the leaflet engagement frame is devoid of any other rungs of struts other than the engagement frame first rung.
Example 183. The valve assembly of any example herein, particularly any one of examples 180 to 182, further comprising an outer skirt comprising an outer skirt outflow end and an outer skirt inflow end, wherein the outer skirt is mounted over an outer surface of the valve assembly such that the outer skirt outflow end is distal to the distally extending spikes.
Example 184. The valve assembly of any example herein, particularly example 180, wherein the one or more rungs of struts of the leaflet engagement frame comprises at least one additional rung of struts distal to the engagement frame first rung, and wherein the leaflet engagement frame further comprises a plurality of proximally extending spikes, extending in the proximal direction from the engagement frame struts of the additional rung of struts.
Example 185. The valve assembly of any example herein, particularly example 184, wherein the proximally extending spikes are extending at a sharp angle β radially outward and in the proximal direction from the additional rung of struts.
Example 186. The valve assembly of any example herein, particularly example 184 or 185, wherein the at least one additional rung of struts comprising the proximally extending spikes is an engagement frame second rung, and wherein the leaflet engagement frame is devoid of any other rungs of struts other than the engagement frame first rung and the engagement frame second rung.
Example 187. The valve assembly of any example herein, particularly example 186, further comprising an outer skirt comprising an outer skirt outflow end and an outer skirt inflow end, wherein the outer skirt is mounted over an outer surface of the valve assembly such that the outer skirt outflow end is distal to the distally extending spikes and the proximally
Example 188. The valve assembly of any example herein, particularly example 184 or 185, wherein the one or more rungs of struts of the leaflet engagement frame further comprises one or more intermediate rungs of struts disposed between the rungs of struts that include the distally extending spikes and the proximally extending spikes, wherein any one of the one or more intermediate rungs of struts is devoid of spikes.
Example 189. The valve assembly of any example herein, particularly example 188, further comprising an outer skirt comprising an outer skirt outflow end and an outer skirt inflow end, wherein the outer skirt is mounted over an outer surface of the valve assembly such that the outer skirt outflow end is distal to the distally extending spikes, and the outer skirt inflow end is proximal to the proximally extending spikes.
Example 190. The valve assembly of any example herein, particularly any one of examples 184 to 189, wherein each proximally extending spike terminates with a sharp distal tip.
Example 191. The valve assembly of any example herein, particularly example 185, wherein the angle β is in the range of 10-80 degrees.
Example 192. The valve assembly of any example herein, particularly example 191, wherein the angle β is in the range of 20-70 degrees.
Example 193. The valve assembly of any example herein, particularly example 192, wherein the angle β is in the range of 30-60 degrees.
Example 194. The valve assembly of any example herein, particularly any one of examples 180 to 193, wherein each distally extending spike terminates with a sharp distal tip.
Example 195. The valve assembly of any example herein, particularly example 181, wherein the angle α is in the range of 10-80 degrees.
Example 196. The valve assembly of any example herein, particularly example 195, wherein the angle α is in the range of 20-70 degrees.
Example 97. The valve assembly of any example herein, particularly example 196, wherein the angle α is in the range of 30-60 degrees.
Example 198. The valve assembly of any example herein, particularly any one of examples 180 to 197, wherein the plurality of struts of the valve frame define a plurality of rungs of struts, comprising:
a valve frame outflow rung;
a valve frame inflow rung; and
at least one valve frame intermediate rung disposed between the valve frame outflow rung and the valve frame inflow rung,
wherein the struts of the engagement frame first rung are aligned with struts of a valve frame intermediate rung which is distal to the valve frame outflow rung.
Example 199. The valve assembly of any example herein, particularly example 198, wherein the at least one valve frame intermediate rung comprises: a valve frame first intermediate rung, a valve frame second intermediate rungs, and a valve frame third intermediate rungs, wherein the struts of the valve frame further comprise valve frame distal axial struts interconnecting struts of the valve frame inflow rung with struts of the valve frame third intermediate rung, and wherein the struts of the engagement frame first rung are aligned with the struts of the valve frame first intermediate rung.
Example 200. The valve assembly of any example herein, particularly example 198, wherein the at least one valve frame intermediate rung comprises a valve frame first intermediate rung and a valve frame second intermediate rung, wherein the struts of the engagement frame first rung are aligned with the struts of the valve frame first intermediate rung.
Example 201. The valve assembly of any example herein, particularly example 200, wherein the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.

It is appreciated that certain features of the disclosed technology, which are, for clarity, described in the context of separate examples, may also be provided in combination in a single example. Conversely, various features of the disclosed technology, which are, for brevity, described in the context of a single example, may also be provided separately or in any suitable sub-combination or as suitable in any other described example of the disclosed technology. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A valve assembly comprising:
a prosthetic valve comprising:
a valve frame comprising a plurality of struts, wherein the valve frame is movable between a radially compressed state and a radially expanded state;
a leaflet assembly mounted within the valve frame and comprising a plurality of leaflets configured to regulate flow through the prosthetic valve; and
a leaflet engagement frame which is disposed radially outwardly from, and is coupled to, the valve frame, the leaflet engagement frame comprising:
a plurality of engagement frame struts defining one or more rungs of struts, wherein the one or more rungs of struts comprises an engagement frame first rung, and wherein the engagement frame first rung further comprises a plurality of distally extending spikes that extend in a distal direction from its engagement frame struts;
wherein the plurality of distally extending spikes is configured to engage with one or more native or host leaflets positioned outside the valve assembly when the valve assembly is in a partially expanded state, and to distally fold the engaged native or host leaflets when the valve assembly is further expanded.

2. The valve assembly of claim 1, wherein the plurality of distally extending spikes extend at an angle α radially outward and in the distal direction from the respective struts of the engagement frame first rung.

3. The valve assembly of claim 1, wherein the leaflet engagement frame is devoid of any other rungs of struts other than the engagement frame first rung.

4. The valve assembly of claim 1, wherein the one or more rungs of struts of the leaflet engagement frame comprises at least one additional rung of struts distal to the engagement frame first rung, and wherein the leaflet engagement frame further comprises a plurality of proximally extending spikes, extending in the proximal direction from the engagement frame struts of the additional rung of struts.

5. The valve assembly of claim 4, wherein the engagement frame struts of the additional rung of struts are devoid of distally extending spikes.

6. The valve assembly of claim 4, wherein the plurality of proximally extending spikes extend at an angle β radially outward and in the proximal direction from the additional rung of struts.

7. The valve assembly of claim 4, wherein the at least one additional rung of struts comprising the proximally extending spikes is an engagement frame second rung, and wherein the leaflet engagement frame is devoid of any other rungs of struts other than the engagement frame first rung and the engagement frame second rung.

8. The valve assembly of claim 4, wherein the one or more rungs of struts of the leaflet engagement frame further comprises one or more intermediate rungs of struts disposed between the rungs of struts that include the distally extending spikes and the proximally extending spikes, wherein any one of the one or more intermediate rungs of struts is devoid of spikes.

9. A valve assembly comprising:
a prosthetic valve comprising:
a valve frame movable between a radially compressed state and a radially expanded state, the valve frame comprising a plurality of frame rungs, each frame rung comprising interconnected struts, the plurality of frame rungs comprising:
a valve frame outflow rung, which is proximal to any other rung of the plurality of frame rungs;
a valve frame inflow rung, which is distal to any other rung of the plurality of frame rungs; and
at least one valve frame intermediate rung disposed between the valve frame outflow rung and the valve frame inflow rung, the at least one valve frame intermediate rung comprising a valve frame first intermediate rung,
a leaflet assembly mounted within the valve frame and comprising a plurality of leaflets configured to regulate flow through the prosthetic valve; and
at least one proximal leaflet engagement wire wrapped around struts of the at least one valve frame intermediate rung,
wherein the at least one proximal leaflet engagement wire is configured to engage one or more native or host leaflets positioned outside the valve assembly when the valve assembly is in a partially expanded state, and to distally fold the engaged native or host leaflets when the valve assembly is further expanded.

10. The valve assembly of claim 9, wherein the at least one valve frame intermediate rung around which the at least one proximal leaflet engagement wire is wrapped, comprises a single valve frame intermediate rung.

11. The valve assembly of claim 10, wherein all other frame rungs are devoid of engagement wires wrapped there-over.

12. The valve assembly of claim 10, further comprising at least one distal leaflet engagement wire wrapped around struts of at least one other rung of the plurality of frame rungs, wherein the at least one other rung is distal to the proximal leaflet engagement wire.

13. The valve assembly of claim 12, wherein the at least one other rung comprises a single rung of the plurality of frame rungs.

14. The valve assembly of claim 13, wherein all other valve frame rungs, except the single rung around which the proximal leaflet engagement wire is wrapped and the single rung around which the distal leaflet engagement wire is wrapped, are devoid of engagement wires wrapped there-over.

15. The valve assembly of claim 12, wherein the at least one other rung around which the engagement wire is wrapped comprises the valve frame inflow rung.

16. The valve assembly of claim 12, wherein the at least one valve frame intermediate rung further comprises a valve frame second intermediate rung, and wherein the at least one other rung around which the distal leaflet engagement wire is wrapped comprises the valve frame second intermediate rung.

17. The valve assembly of claim 16, wherein the axial distance between each strut of the valve frame first intermediate rung and the respective strut of the valve frame outflow rung, at any specific circumferential location around the valve frame, is greater than the axial distance between the strut of the valve frame second intermediate rung and the respective strut of the valve frame inflow rung at the same circumferential location.

18. A valve assembly comprising:
a prosthetic valve comprising:
a valve frame movable between a radially compressed state and a radially expanded state;
a leaflet assembly mounted within the valve frame and comprising a plurality of leaflets configured to regulate flow through the prosthetic valve;
an outer skirt attached to the valve frame and comprising a floating fiber portion disposed between an outer skirt outflow end and an outer skirt inflow end, the floating fiber portion comprising a plurality of filaments; and
a leaflet engagement frame which is disposed around, and is coupled to, the outer skirt, the leaflet engagement frame comprising at least one rung of engagement frame struts, and a plurality of spikes extending from the engagement frame struts;
wherein the spikes are configured to engage with a native tissue outside the valve assembly to irritate the native tissue.

19. The valve assembly of claim 18, wherein the outer skirt further comprises an outflow woven portion and an inflow woven portion, and wherein the floating fiber portion is bounded between the outflow woven portion and the inflow woven portion.

20. The valve assembly of claim 19, wherein the leaflet engagement frame is disposed between the outflow woven portion and the inflow woven portion.

21. The valve assembly of claim 18, wherein the plurality of filaments curves radially outward in the radially expanded state.

22. The valve assembly of claim 18, wherein the at least one rung of engagement frame struts comprises two rungs of engagement frame struts.

23. The valve assembly of claim 18, wherein the spikes extend along a circumferential plane defined by the engagement frame struts.

24. The valve assembly of claim 18, wherein the spikes extend at an angle radially outward and in an axial direction from the engagement frame struts.
